(19) United States
(12) Patent Application Publication
(10) Pub. No.: US 2022/0088024 A1
(43) Pub. Date: Mar. 24, 2022

(54) METHODS FOR TREATING CANCER USING TOZADENANT

(71) Applicant: Biotie Therapies, Inc., Ardsley, NY (US)

(72) Inventor: Cory Kostrub, Belmont, CA (US)

(73) Assignee: Biotie Therapies, Inc., Ardsley, NY (US)

(21) Appl. No.: 17/414,915

(22) PCT Filed: Dec. 19, 2019
(86) PCT No.: PCT/US2019/067610

§ 371 Date: Jun. 16, 2021

Related U.S. Application Data
(60) Provisional application No. 62/783,186, filed on Dec. 20, 2018.
(60) Provisional application No. 62/788,755, filed on Jan. 4, 2019.

(51) Int. Cl. A61K 31/5377; C07K 16/28; A61P 35/04

(52) U.S. Cl. CPC A61K 31/5377; A61P 35/04; C07K 16/2818

(57) ABSTRACT

Provided herein are methods and compositions for treatment of patients with cancer using tozadenant. Methods include monotherapy as well as combination therapy.

### 1. CROSS -REFERENCE TO RELATED APPLICATIONS

This application claims the benefit of U.S. Provisional Application No. 62/783,186, filed on Dec. 20, 2018 and U.S. Provisional Application No. 62/788,755, filed on Jan. 4, 2019, each of which is incorporated by reference herein in its entirety.

### 2. FIELD OF INVENTION

The invention relates to treatment of patients with cancer using tozadenant.

### 3. BACKGROUND

### 3.1 Cancer and Adensoine A_{2A} Receptor Antagonists

There is scientific literature demonstrating the role of adenosine in immune system regulation, particularly as a local immunosuppressant. Such “immune checkpoint” pathways are critical to prevent runaway immune system activation and autoimmunity

The adenosine system works with other immune checkpoint receptors such as CTLA-4 and PD-1 to prevent over-activation of the immune system and allow discrimination of self tissue from pathogens.

Of the adenosine receptors, the adenosine A₂ₐ receptor has been identified as the most relevant to immunomodulation based upon its expression pattern in immune cells and high affinity for adenosine. Several publications have described A₂ₐ as potential oncology target. These publications include the following: Leone et al., 2015. Review on A₂ₐ antagonists as immunotherapy for cancer, Computational and Structural Biotechnology Journal 13 (2015, 265-272; Sitkovsky et al., 2008. Review on A₂ₐ antagonists as immunotherapy for cancer, British Journal of Pharmacology (2008) 153, 5457-5464; Pennock and Chow, 2015. Review of checkpoint inhibitors in cancer treatment, The Oncologist 2015; 20:812-822; Cekic and Linden, 2015. A₂ₐ receptors and T-cell regulation in tumours, Cancer Res, 74(24): 7239-7249; Mittal et al., 2014. Anti-metastatic effect of PD-1 and A₂ₐ blocking, Cancer Res, 74(14) 3652-3658; and Ohta et al., 2006. First description of A₂ₐ antagonists as anti-tumour agents, PNAS 103(35): 13132-13137.

A₂ₐ receptor antagonists such as SCH 58261 and CPI444 have demonstrated activity in mouse tumor models, both as monotherapy and in combination with anti-PD-1 agents. SCH 58261 demonstrated activity as monotherapy in a mouse model (see Hammerl D. et al., “Intratumoral injection of microparticles containing the A_{2A} receptor antagonist SCH58261 slow tumor growth and metastasis more effectively than system drug administration,” Abstract in Purinergic Signal. 1019 (2016)). CPI444 was effective in mouse tumor models alone and in combination with an anti-PD-1 agent, and is being investigated in clinical trials for cancer (see Willingham et al., Abstract PRO4: Second CRI-CIMT-EATI-AACR International Cancer Immunotherapy Conference: Translating Science into Survival; September 25-28, 2016; New York, N.Y.).

### 3.2 Tozadenant and A2a

Tozadenant (SYN-115) is a potent and selective A₂ₐ receptor antagonist, and was tested in human clinical trials for the treatment of Parkinson's Disease (see U.S. Pat. No. 7,368,446 and Hauser et al, Lancet Neurol 2014, 13:767-776).

It has been shown that anti-tumor immune responses evoked by anti-PD-1 could be enhanced by SYN-115 in mouse tumor models (Beavis et al. 2015, Cancer Immunol. Res, 3(5) May 2015, 506-517).

Citation of a reference herein shall not be construed as an admission that such is prior art to the present invention.

### 4. SUMMARY OF THE INVENTION

Provided herein is a method for treating cancer in a human patient in need thereof, comprising orally administering to the patient tozadenant or a pharmaceutically acceptable salt of tozadenant. In particular, disclosed herein is a method of treating cancer in a human patient in need thereof, said method comprising orally administering to the patient a maximum daily dose in the range of about 0.5 to about 1.0 mg of tozadenant or a molar-equivalent amount thereof of a pharmaceutically acceptable salt of tozadenant.

In one embodiment, the administering of said maximum daily dose is daily. In another embodiment, the administering of said maximum daily dose is every other day. In one embodiment, the administering of said maximum daily dose is every 3-4 days. In another embodiment, the administering of said maximum daily dose is weekly. In one embodiment, the administering of said maximum daily dose is every other week.

In one embodiment, the dose is about 0.5 mg. In one embodiment, the dose is about 0.6 mg. In one embodiment, the dose is about 0.7 mg. In one embodiment, the dose is about 0.8 mg. In one embodiment, the dose is about 0.9 mg. In one embodiment, the dose is about 1.0 mg. In one embodiment, the dose is 1.0 mg. In one embodiment, the dose is 0.5 mg.

In one embodiment, the dose is administered in two separate administrations on the same day. In another embodiment, the separate administrations are administered about once every 12 hours. In one embodiment, the separate administrations are each about 50% of the daily dose.

In one embodiment, the cancer is a solid tumor. In one embodiment, the cancer is breast cancer. In one embodiment, the cancer is colon cancer. In one embodiment, the cancer is lung cancer. In one embodiment, the cancer is melanoma.

In one embodiment, the tozadenant or a pharmaceutically acceptable salt thereof is formulated as a capsule, a pill or a tablet. In another embodiment, the tozadenant or a pharmaceutically acceptable salt thereof is formulated as a tablet.

In a specific embodiment, tozadenant is the only cancer therapeutic administered to said human patient during the course of treatment with tozadenant.

In one embodiment, the method comprises concomitantly treating the patient with a PD-1 inhibitor. In another embodiment, the PD-1 inhibitor is an anti-PD-1 monoclonal antibody.

In one embodiment, the tozadenant or a pharmaceutically acceptable salt thereof is combined with a pharmaceutically acceptable carrier in a pharmaceutical composition. In another embodiment, tozadenant is administered.

Provided herein is a unit dosage pharmaceutical composition comprising about 0.5 to about 1.0 mg of tozadenant or a molar-equivalent amount thereof of a pharmaceutically acceptable salt of tozadenant, and a pharmaceutically acceptable carrier. In one embodiment, the unit dosage pharmaceutical composition comprises about 0.5 mg of tozadenant or a molar-equivalent amount thereof of a pharmaceutically acceptable salt of tozadenant. In one embodiment, the unit dosage pharmaceutical composition comprises about 0.6 mg of tozadenant or a molar-equivalent amount thereof of a pharmaceutically acceptable salt of tozadenant. In one embodiment, the unit dosage pharmaceutical composition comprises about 0.7 mg of tozadenant or a molar-equivalent amount thereof of a pharmaceutically acceptable salt of tozadenant. In one embodiment, the unit dosage pharmaceutical composition comprises about 0.8 mg of tozadenant or a molar-equivalent amount thereof of a pharmaceutically acceptable salt of tozadenant. In one embodiment, the unit dosage pharmaceutical composition comprises about 0.9 mg of tozadenant or a molar-equivalent amount thereof of a pharmaceutically acceptable salt of tozadenant. In one embodiment, the unit dosage pharmaceutical composition comprises about 1.0 mg of tozadenant or a molar-equivalent amount thereof of a pharmaceutically acceptable salt of tozadenant. In one embodiment, the unit dosage pharmaceutical composition comprises 1.0 mg of tozadenant or a molar-equivalent amount thereof of a pharmaceutically acceptable salt of tozadenant. In one embodiment, the unit dosage pharmaceutical composition comprises 0.5 mg of tozadenant or a molar-equivalent amount thereof of a pharmaceutically acceptable salt of tozadenant.

In one embodiment, the unit dosage pharmaceutical composition is formulated as a capsule, a pill or a tablet. In one embodiment, the unit dosage pharmaceutical composition is formulated as a tablet.

Provided herein is a kit comprising in one or more containers a plurality of a unit dosage pharmaceutical composition described herein.

Provided herein is a method of treating cancer in a human patient in need thereof, said method comprising orally administering to the patient a unit dosage pharmaceutical composition described herein.

In one embodiment, the administering of said unit dosage pharmaceutical composition is once daily. In one embodiment, the administering of said unit dosage pharmaceutical composition is once every other day. In one embodiment, the administering of said unit dosage pharmaceutical composition is once per week. In one embodiment, the administering of said unit dosage pharmaceutical composition is once every 3-4 days. In one embodiment, the administering of said unit dosage pharmaceutical composition is once every other week.

In one embodiment, the cancer is a solid tumor. In one embodiment, the cancer is breast cancer. In one embodiment, the cancer is colon cancer. In one embodiment, the cancer is lung cancer. In one embodiment, the cancer is melanoma.

In one embodiment, tozadenant is the only cancer therapeutic administered to said human patient during the course of treatment with tozadenant.

In one embodiment, the method further comprises concomitantly treating the patient with a PD-1 inhibitor. In one embodiment, the PD-1 inhibitor is an anti-PD-1 monoclonal antibody.

Provided herein is the use of a maximum daily dose in the range of about 0.5 to about 1.0 mg of tozadenant or a molar-equivalent amount thereof of a pharmaceutically acceptable salt of tozadenant for the manufacture of a medicament for treating cancer in a human patient, preferably said medicament being formulated for oral administration.

Provided herein is tozadenant or a pharmaceutically acceptable salt thereof for use in treating cancer in a human at a maximum daily oral dose in the range of about 0.5 to about 1.0 mg of tozadenant or a molar-equivalent amount thereof of a pharmaceutically acceptable salt of tozadenant.

Provided herein is a method of treating cancer in a human patient in need thereof, said method comprising orally administering to the patient an amount of tozadenant or a pharmaceutically acceptable salt thereof that provides an AUC at steady state of about 71 hr*ng/mL to about 106 hr*ng/mL in the patient.

In one embodiment, the AUC at steady state is about 106 hr*ng/mL. In one embodiment, the AUC at steady state is about 71 hr*ng/mL.

### 4.1 Terminology

In order to provide a clear and consistent understanding of the specification and claims, the following definitions are provided:

As used herein, “about” means + or −10%.

By “pharmaceutically acceptable,” it is meant the carrier, diluent or excipient must be compatible with the other ingredients of the formulation and not prohibited for human or veterinary administration (as the case may be) by a regulatory agency such as the Food and Drug Administration or European Medicines Agency.

The term “pharmaceutically acceptable salt(s),” with reference to tozadenant, as used herein, refers to a salt prepared from a pharmaceutically acceptable non-toxic acid or base, including an inorganic acid or base, or an organic acid or base. In one embodiment, the pharmaceutically acceptable salt is prepared from a pharmaceutically acceptable non-toxic acid which can be an inorganic or organic acid. In one embodiment, non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. In one embodiment, the non-toxic acid is hydrochloric acid. Suitable pharmaceutically acceptable salts will be apparent to those skilled in the art and include those described in S. M. Barge et al., “Pharmaceutical Salts,” 1977, J. Pharm. Sci. 66: 1-19, which is incorporated herein by reference in its entirety.

Other terms and/or abbreviations are provided below

| Abbreviation or | |
|---|---|
| Specialist Term | Explanation |
| b.i.d. (bid) | Twice daily |
| CFR | Code of Federal Regulations |
| Cₘₐₓ | Maximum measured plasma concentration |
| Cₘₐₓₛₛ | Maximum measured plasma concentration |
| | at steady state |
| Cₘᵢₙ | Minimum measured plasma concentration |
| Cₘᵢₙₛₛ | Minimum measured plasma concentration |
| | at steady state |
| g, kg, mg, | Gram, kilogram, milligram, microgram, |
| μg, ng | nanogram |
| GLP | Good Laboratory Practice |
| h, hr | Hour |
| HPLC | High performance liquid chromatography |
| IV, i.v., or iv | Intravenous |
| L, mL | Liter, milliliter |
| LCMS, | Liquid chromatography/mass spectrometry |
| LC/MS/MS | |
| Min | Minute |
| mM, μM | Millimolar, micromolar |
| NF | National Formulary |
| PD | Parkinson's disease |
| p.o. | Oral |
| q.d. (qd) | Once a day |
| SYN-115 | Tozadenant |
| t.i.d. (tid) | Three times daily |
| Tₘₐₓ | Time of the maximum measured plasma |
| | concentration post-dose |
| Tozadenant | 4-Hydroxy-4-methyl-piperidine-1- |
| | carboxylic acid (4-methoxy-7-morpholin- |
| | 4-yl-benzothiazol-2-yl)-amide |
| USP | United States Pharmacopeia |

### 5. BRIEF DESCRIPTION OF DRAWINGS

FIG. 1 depicts effect of the treatment described in Example 1 on tumor area.

FIG. 2 depicts effect of the treatment described in Example 2 on tumor area.

FIG. 3 depicts effect of the treatment described in Example 3 on tumor area.

FIG. 4 depicts effect of the treatment described in Example 4 on tumor area.

FIG. 5 depicts effect of the treatment described in Example 4 on percent survival.

FIG. 6 depicts effect of the treatment described in Example 5 on tumor area.

FIG. 7 depicts effect of the treatment described in Example 5 on percent survival.

FIG. 8 depicts effect of the treatment described in Example 6 on tumor area.

FIG. 9 depicts effect of the treatment described in Example 6 on percent survival.

FIG. 10 depicts effect of the treatment described in Example 7 on tumor area.

FIG. 11 depicts effect of the treatment described in Example 7 on percent survival.

FIG. 12 depicts effect of the low dose and high dose tozadenant treatments described in Example 7 on percent survival.

FIG. 13 depicts effect of the treatment with various dose levels of tozadenant described in Example 7 on tumor area on day 14 of the treatment.

FIG. 14 depicts effect of the treatment described in Example 8 on metastases.

FIG. 15 depicts effect of the treatment described in Example 9 on metastases.

FIG. 16 depicts effect of the treatment described in Example 10 on metastases.

FIG. 17: FACS Panel used in Examples 14-15.

FIG. 18: Tumor weights from Examples 14-15.

FIG. 19: Number of CD8+ T cells per mg from Examples 14-15.

FIG. 20: Number of CD8+ foxp3− per mg from Examples 14-15.

FIG. 21: CD4+ foxp3+ per mg from Examples 14-15.

FIG. 22: CD8:CD4+Foxp3+ ratio (CD8:Treg ratio) from Examples 14-15.

FIG. 23: Percent of CD8+ cells expressing IFNy from Examples 14-15.

FIG. 24: Percent of CD4+ foxp3− cells expressing IFNy from Examples 14-15.

FIG. 25: Percent of CD8+ cells expressing TNFa from Examples 14-15.

FIG. 26: Percent of CD4+foxp3− cells expressing TNFa from Examples 14-15.

FIG. 27: Percent of CD8+ cells expressing Ki67 from Examples 14-15.

FIG. 28: Percent of CD4+foxp3− cells expressing Ki67 from Examples 14-15.

FIG. 29: Percent of CD8+ cells expressing CD62L from Examples 14-15.

FIG. 30: Percent of CD4+ foxp3− cells CD62L− from Examples 14-15.

FIG. 31: Il7R expression on CD8+t cells from Examples 14-15.

FIG. 32: Expression of IL7R on CD4+foxp3− cells foxp3− from Examples 14-15.

FIG. 33: Percent of CD8+ T cells expressing PD-1 from Examples 14-15.

FIG. 34: Percent of CD4+ foxp3− cells expressing PD-1 from Examples 14-15.

FIG. 35: Numbers of CD8 T cells in Draining lymph node from Examples 14-15.

FIG. 36: Numbers of CD4+foxp3− T cells in Draining lymph node from Examples 14-15.

FIG. 37: Numbers of CD4+foxp3− T cells in Draining lymph node from Examples 14-15.

FIG. 38: Percent CD8+ T cells in Draining lymph node from Examples 14-15.

FIG. 39: Percent CD4+foxp3− T cells in Draining lymph node from Examples 14-15.

FIG. 40: Percent CD4+foxp3+ T cells in Draining lymph node from Examples 14-15.

FIG. 41: CD8:CD4+Foxp3+ ratio in DLN (CD8:Treg ratio) from Examples 14-15.

FIG. 42: Percent of CD8+ cells expressing IFNy from Examples 14-15.

FIG. 43: Percent of CD4+foxp3− cells expressing IFNy from Examples 14-15.

FIG. 44: Percent of CD8 cells expressing TNF from Examples 14-15.

FIG. 45: Percent of CD4+foxp3− cells expressing TNF from Examples 14-15.

FIG. 46: Percent of CD8 cells which are CD62L negative from Examples 14-15.

FIG. 47: Percent of CD4+foxp3− cells which are CD62L negative from Examples 14-15.

### 6. DETAILED DESCRIPTION

### 6.1 Tozadenant for Use in the Methods of the Invention

Provided herein is a method of treating cancer in a human patient in need thereof, said method comprising orally administering to the patient a maximum daily dose in the range of about 0.6 to about 0.9 mg of tozadenant or a molar-equivalent amount thereof of a pharmaceutically acceptable salt of tozadenant.

Tozadenant is also known as SYN-115, or by its chemical name 4-hydroxy-4-methyl-piperidine-1-carboxylic acid (4-methoxy-7-morpholin-4-yl-benzothiazol-2-yl)-amide. Tozadenant is disclosed in U.S. Pat. No. 7,368,446 and U.S. Patent Application Publication No. 2018/0303843, each of which is herein incorporated by reference in their entirety. Tozadenant has the following chemical structure:

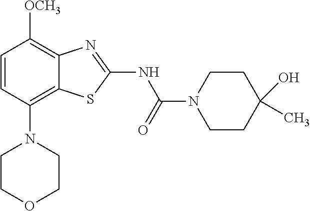 [SMILES: COc1ccc(N2CCOCC2)c2sc(NC(=O)N3CCC(C)(O)CC3)nc12]

As will be appreciated, a pharmaceutically acceptable salt of tozadenant may be used instead of tozadenant in any or all of the methods of treating cancer disclosed herein. Thus, in specific embodiments, a pharmaceutically acceptable salt of tozadenant is used in the methods of treating a patient with cancer described herein. These salts can be prepared, for example, in situ during the final isolation and purification of the compounds or by separately reacting the purified compound in its free base form with a suitable organic or inorganic acid and isolating the salt thus formed. In some embodiments, the pharmaceutically acceptable salt of tozadenant is prepared using acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid. In one embodiment, one equivalent of tozadenant, as used herein, may form an acid salt with less than one or with one or more than one equivalent of an acid. For further description of pharmaceutically acceptable salts that can be used in the methods described herein see, for example, S. M. Barge et al., “Pharmaceutical Salts,” 1977, J. Pharm. Sci. 66:1-19, which is incorporated herein by reference in its entirety.

In certain embodiments, tozadenant itself, and not a pharmaceutically acceptable salt thereof, is used in any of the methods of treating cancer described herein.

### 6.2 Cancers Treated in Accordance with the Invention

Provided herein is a method of treating cancer in a human patient in need thereof, said method comprising orally administering to the patient a maximum daily dose in the range of about 0.5 to about 1.0 mg of tozadenant or a molar-equivalent amount thereof of a pharmaceutically acceptable salt of tozadenant.

Provided herein is a method of treating cancer in a human patient in need thereof, said method comprising orally administering to the patient a unit dosage pharmaceutical composition of tozadenant described herein.

In one embodiment, the administering of said unit dosage pharmaceutical composition is once daily. In one embodiment, the administering of said unit dosage pharmaceutical composition is once every other day. In one embodiment, the administering of said unit dosage pharmaceutical composition is once per week. In one embodiment, the administering of said unit dosage pharmaceutical composition is once every 3-4 days. In one embodiment, the administering of said unit dosage pharmaceutical composition is once every other week.

In one embodiment, tozadenant is the only cancer therapeutic administered to said human patient during the course of treatment with tozadenant.

In one embodiment, the method further comprises concomitantly treating the patient with a PD-1 inhibitor. In one embodiment, the PD-1 inhibitor is an anti-PD-1 monoclonal antibody.

Types of cancer that can be treated according to the instant invention include but are not limited to bladder cancer, breast cancer, colon and rectal cancer, endometrial cancer, kidney cancer, leukemia, liver cancer, lung cancer, melanoma, non-Hodgkin lymphoma, pancreatic cancer, prostate cancer, and thyroid cancer.

In one embodiment, the cancer is a tumor. In one embodiment, the cancer is a solid tumor.

In a specific embodiment is a method for achieving complete eradication of a tumor, said method comprising orally administering to a human tumor patient a maximum daily dose in the range of about 0.5 to about 1.0 mg of tozadenant or a molar-equivalent amount thereof of a pharmaceutically acceptable salt of tozadenant. In one embodiment, eradication of the tumor is achieved.

In a specific embodiment is a method for achieving complete eradication of a malignant tumor, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In one embodiment, eradication of the malignant tumor is achieved.

In a specific embodiment is a method for inhibiting malignant tumor growth, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In one embodiment, an inhibition of malignant tumor growth is achieved.

In a specific embodiment is a method for reducing malignant tumor size, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In one embodiment, a reduction of malignant tumor size is achieved.

In a specific embodiment is a method for reducing malignant tumor burden, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In one embodiment, a reduction of malignant tumor burden is achieved.

In a specific embodiment is a method for eliminating malignant tumor burden, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In one embodiment, an elimination of malignant tumor burden is achieved.

In a specific embodiment is a method for inhibiting metastasis of a malignant tumor, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In a specific embodiment, an inhibition of metastasis of the malignant tumor is achieved.

In a specific embodiment is a method for reducing the extent of metastasis of a malignant tumor, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In a specific embodiment, a reduction of the extent of metastasis of the malignant tumor is achieved.

In a specific embodiment is a method for eliminating metastasis of a malignant tumor, said method comprising orally administering to a human patient having a malignant tumor a maximum daily dose in the range of about 0.5 to about 1.0 mg of tozadenant or a molar-equivalent amount thereof of a pharmaceutically acceptable salt of tozadenant. In a specific embodiment, an elimination of metastasis of the malignant tumor is achieved.

In one embodiment, the cancer is breast cancer. In one embodiment, the cancer is colon cancer. In one embodiment, the cancer is lung cancer. In one embodiment, the cancer is melanoma.

In a specific embodiment, the method of treating cancer in a human patient in need thereof can be a method for doing one or more of the following: preventing progression of the cancer, or eradicating the cancer, or inhibiting, reducing the extent of, or eliminating metastasis, or decreasing tumor burden, or ameliorating, alleviating, palliating, decreasing, or preventing one or more symptoms of the cancer.

### 6.3 Dosing

6.3.1 Calculation of Human Dose

As described in Example 13, the dose for human use for tozadenant was determined by calculations based on efficacious mouse doses by use of relevant mouse and human tozadenant PK data.

6.3.2 Dosing Regimens

A dosing regimen described herein can be used to carry out the therapeutic methods of the invention.

In a particular embodiment, the method in accordance with the invention comprises a method for treating cancer in a human patient in need thereof, comprising orally administering to the patient tozadenant or a pharmaceutically acceptable salt of tozadenant. In a particular embodiment, the method is a method of treating cancer in a human patient in need thereof, said method comprising orally administering to the patient a maximum daily dose in the range of about 0.5 to about 1.0 mg of tozadenant or a molar-equivalent amount thereof of a pharmaceutically acceptable salt of tozadenant.

In one embodiment, the administering of said maximum daily dose is daily. In another embodiment, the administering of said maximum daily dose is every other day. In one embodiment, the administering of said maximum daily dose is every 3-4 days. In another embodiment, the administering of said maximum daily dose is weekly. In one embodiment, the administering of said maximum daily dose is every other week.

In one embodiment, the dose is about 0.5 mg. In one embodiment, the dose is about 0.6 mg. In one embodiment, the dose is about 0.7 mg. In one embodiment, the dose is about 0.8 mg. In one embodiment, the dose is about 0.9 mg. In one embodiment, the dose is about 1.0 mg. In one embodiment, the dose is 1.0 mg. In one embodiment, the dose is 0.5 mg.

In one embodiment, the dose is administered in two separate administrations on the same day. In another embodiment, the separate administrations are administered about once every 12 hours. In one embodiment, the separate administrations are each about 50% of the daily dose.

In some embodiments, a patient is treated in accordance with the methods described herein for a period of time that is, e.g., for at least 1 week, at least 2 weeks, at least 3 weeks, at least 4 weeks, at least 5 weeks, at least 6 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, at least 5 years, at least 10 years, or more than 5 or 10 years. In certain embodiments, the treatment regimen (a particular dose and frequency of administration, which can be selected from any described herein) is stable over a period of time, e.g., for at least 1 week, at least 2 weeks, at least 3 weeks, at least 1 month, at least 2 months, at least 3 months, at least 4 months, at least 6 months, or at least 1 year.

In one embodiment, tozadenant is administered to a human subject so as to provide an AUC at steady state in the range of about 71 hr*ng/mL to about 106 hr*ng/mL. Accordingly, the invention provides a method of treating cancer in a human patient in need thereof, said method comprising orally administering to the patient an amount of tozadenant or a pharmaceutically acceptable salt thereof that provides an AUC at steady state of about 71 hr*ng/mL to about 106 hr*ng/mL in the patient.

In one embodiment, tozadenant is administered to a human subject so as to provide an AUC at steady state of about 106 hr*ng/mL. Accordingly, the invention provides a method of treating cancer in a human patient in need thereof, said method comprising orally administering to the patient an amount of tozadenant or a pharmaceutically acceptable salt thereof that provides an AUC at steady state of about 106 hr*ng/mL in the patient.

In one embodiment, tozadenant is administered to a human subject so as to provide an AUC at steady state of about 71 hr*ng/mL. Accordingly, the invention provides a method of treating cancer in a human patient in need thereof, said method comprising orally administering to the patient an amount of tozadenant or a pharmaceutically acceptable salt thereof that provides an AUC at steady state of about 71 hr*ng/mL in the patient.

Provided herein is the use of a maximum daily dose in the range of about 0.6 to about 0.9 mg of tozadenant or a molar-equivalent amount thereof of a pharmaceutically acceptable salt of tozadenant for the manufacture of a medicament treating cancer in a human patient in need thereof, said composition formulated for oral administration.

Provided herein is a maximum daily oral dose in the range of about 0.6 to about 0.9 mg of tozadenant or a molar-equivalent amount thereof of a pharmaceutically acceptable salt of tozadenant for use in treating cancer in a human patient in need thereof.

In a preferred embodiment, tozadenant or a pharmaceutically acceptable salt thereof is administered in an immediate release composition.

### 6.4 Pharmaceutical Compositions

The invention also provides pharmaceutical compositions comprising tozadenant or a pharmaceutically acceptable salt thereof as described herein. Such pharmaceutical compositions can comprise an amount (e.g., a therapeutically effective amount) of tozadenant or a pharmaceutically acceptable salt thereof and a pharmaceutically acceptable carrier. In one embodiment, the pharmaceutical composition is suitable for oral administration and can be, for example, a pill, tablet or capsule. The pharmaceutical compositions of the invention are administered to a patient for any of the uses described herein.

Tozadenant or a pharmaceutically acceptable salt thereof is preferably administered to a patient orally or parenterally in the conventional form of preparations, such as capsules, microcapsules, tablets, granules, powder, troches, pills, suppositories, injections, suspensions, or syrups. Suitable formulations can be prepared by methods commonly employed using conventional, organic or inorganic additives, such as one or more of: an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). In some embodiments, suitable formulations of tozadenant or a pharmaceutically acceptable salt thereof can be prepared using one, two, three or more, or all, of the following additives: colloidal silicon dioxide, hydroxypropyl methylcellulose, magnesium stearate, microcrystalline cellulose, polyethylene glycol, and titanium dioxide.

A pharmaceutically acceptable carrier or vehicle can comprise an excipient, diluent, or a mixture thereof. In some embodiments, suitable formulations (e.g., suitable formulations such as tablets for oral administration) of tozadenant or a pharmaceutically acceptable salt thereof are prepared using one or more of the following excipients: hydroxypropyl methylcellulose, USP; microcrystalline cellulose, USP; colloidal silicon dioxide, NF; magnesium stearate, USP; and Opadry White.

The amount of tozadenant or a pharmaceutically acceptable salt thereof that is present in the pharmaceutical composition is preferably an amount that will exercise the desired effect.

In a preferred embodiment, tozadenant or a pharmaceutically acceptable salt thereof is administered orally. In a preferred embodiment, tozadenant is administered orally. In some of the embodiments wherein tozadenant or a pharmaceutically acceptable salt thereof is administered orally, the composition is formulated in a form of a tablet, a pill or a capsule. Tozadenant or a pharmaceutically acceptable salt thereof can also be administered intradermally, intramuscularly, intraperitoneally, percutaneously, intravenously, subcutaneously, intranasally, epidurally, sublingually, intracerebrally, intravaginally, transdermally, rectally, by inhalation, or topically to the ears, nose, eyes, or skin. The mode of administration is left to the discretion of the health-care practitioner.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be, e.g., a single tablet or capsule or convenient volume of a liquid.

Capsules can be prepared by any known method, such as mixing tozadenant or a pharmaceutically acceptable salt thereof with a suitable carrier or diluent and filling the proper amount of the mixture in capsules. Carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by known methods such as direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

In one embodiment, the tozadenant or a pharmaceutically acceptable salt thereof is formulated as a capsule, a pill or a tablet. In another embodiment, the tozadenant or a pharmaceutically acceptable salt thereof is formulated as a tablet.

In one embodiment, the tozadenant or a pharmaceutically acceptable salt thereof is combined with a pharmaceutically acceptable carrier in a pharmaceutical composition. In another embodiment, tozadenant is administered.

Provided herein is a unit dosage pharmaceutical composition comprising about 0.5 to about 1.0 mg of tozadenant or a molar-equivalent amount thereof of a pharmaceutically acceptable salt of tozadenant, and a pharmaceutically acceptable carrier. In one embodiment, the unit dosage pharmaceutical composition comprises about 0.5 mg of tozadenant or a molar-equivalent amount thereof of a pharmaceutically acceptable salt of tozadenant. In one embodiment, the unit dosage pharmaceutical composition comprises about 0.6 mg of tozadenant or a molar-equivalent amount thereof of a pharmaceutically acceptable salt of tozadenant. In one embodiment, the unit dosage pharmaceutical composition comprises about 0.7 mg of tozadenant or a molar-equivalent amount thereof of a pharmaceutically acceptable salt of tozadenant. In one embodiment, the unit dosage pharmaceutical composition comprises about 0.8 mg of tozadenant or a molar-equivalent amount thereof of a pharmaceutically acceptable salt of tozadenant. In one embodiment, the unit dosage pharmaceutical composition comprises about 0.9 mg of tozadenant or a molar-equivalent amount thereof of a pharmaceutically acceptable salt of tozadenant. In one embodiment, the unit dosage pharmaceutical composition comprises about 1.0 mg of tozadenant or a molar-equivalent amount thereof of a pharmaceutically acceptable salt of tozadenant. In one embodiment, the unit dosage pharmaceutical composition comprises 1.0 mg of tozadenant or a molar-equivalent amount thereof of a pharmaceutically acceptable salt of tozadenant. In one embodiment, the unit dosage pharmaceutical composition comprises 0.5 mg of tozadenant or a molar-equivalent amount thereof of a pharmaceutically acceptable salt of tozadenant.

In one embodiment, the unit dosage pharmaceutical composition is formulated as a capsule, a pill or a tablet. In one embodiment, the unit dosage pharmaceutical composition is formulated as a tablet.

Provided herein is a kit comprising in one or more containers a plurality of a unit dosage pharmaceutical composition described herein.

In a preferred embodiment, the pharmaceutical composition is an immediate release composition.

### 6.5 Combination Treatments

In a specific embodiment, one can combine tozadenant or a pharmaceutically acceptable salt thereof with one or more other cancer therapeutic agents in concomitant therapy for the treatment of a patient with cancer. Concomitant therapy can be concurrently, simultaneously, or sequentially (before or after). In a specific embodiment, concurrent administration can be over the same treatment period, e.g., on the same day, during the same week, or during the same two-week period, the same month, etc. In some embodiments, tozadenant or a pharmaceutically acceptable salt thereof is administered to a patient concomitantly with one or more additional drugs or therapy. In a specific embodiment, tozadenant or a pharmaceutically acceptable salt thereof is administered concomitantly with another drug or drugs effective for the treatment of cancer. In certain embodiments, the drug(s) administered concomitantly with tozadenant or a pharmaceutically acceptable salt thereof is a PD-1 inhibitor. In another embodiment, the PD-1 inhibitor is an anti-PD-1 monoclonal antibody. In a specific embodiment, tozadenant or a pharmaceutically acceptable salt thereof is administered concomitantly with one or more additional therapies effective for the treatment of cancer. In a specific embodiment, the one or more additional therapies can include, but are not limited to, radiation therapy or surgery. In another embodiemt, the one or more additional therapies is radiation therapy. In another embodiemt, the one or more additional therapies is surgery.

In yet other embodiments, tozadenant or a pharmaceutically acceptable salt thereof is administered to a patient without an additional drug or therapy for cancer, or without one or more of additional treatments for cancer. Thus, in a specific embodiment, tozadenant is administered not in combination with another cancer therapeutic. In a specific embodiment, tozadenant is the only cancer therapeutic administered to said human patient during the course of treatment with tozadenant. As will be clear, “course of treatment” in this context refers to the time period over which tozadenant is administered.

### 7. EXAMPLES

### 7.1 Example 1: Evaluation of Anti-Tumor Immune Responses of SYN-115 in the AT-3ova CD73+ Tumour Cell Mouse Model

### Methods:

The C57BL/6 mouse breast carcinoma cell line AT-3 was obtained from Dr. Trina Stewart (Griffith University, Nathan, QLD, Australia. Tumor cell lines were transduced to express chicken ovalbumin peptide and CD73 as previously described (1, 2, 3). Tumor cells are periodically verified to be Mycoplasma negative by the Victorian Infectious Diseases References Lab (Melbourne, VIC, Australia) by PCR analysis. Tumor cells were grown in DMEM supplemented with 10% FCS, GlutaMAX, and penicillin/streptomycin. 5×10⁵ cells were resuspended in PBS and injected s.c. into C57/BL6 mice in a 100 μL volume. At day 14 tumors were measured and mice were randomized into distinct treatment groups to ensure that each group had tumors of similar size at the onset of treatment. Mice were treated i.p. at days 14, 18, 22, and 26 with either isotype control (2A3; 200 μg/mouse) or anti-PD-1 mAb (RMP1-14, 200 μg/mouse) and where indicated SYN-115 (Batch 71236AA002) at the indicated dose or vehicle control once-daily via oral gavage on days 14 to 29. To calculate a dose of SYN-115, mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

### Results:

Raw Tumor Measurements:

2A3/Vehicle

| Day | Mouse 1 | Mouse 2 | Mouse 3 | Mouse 4 | Mouse 5 | Mouse 6 |
|---|---|---|---|---|---|---|
| 0 | 12.16 | 15.21 | 15.48 | 18.72 | 18.9 | 35.7 |
| 1 | 18.06 | 13.33 | 24 | 28.98 | 23.46 | 44.55 |
| 4 | 25.48 | 22.79 | 37.21 | 40.88 | 35.51 | 53.32 |
| 6 | 29.68 | 19 | 36 | 43.45 | 38.43 | 67.32 |
| 9 | 33 | 24.5 | 42.21 | 45.6 | 46.15 | 72.24 |
| 11 | 34.02 | 42.18 | 55.08 | 53.58 | 56.76 | 85.41 |
| 14 | 39 | 33.39 | 55.3 | 56.84 | 64.24 | 102 |
| 16 | 54.4 | 31.8 | 62.56 | 62.37 | 58.29 | 105.91 |
| 18 | 71.28 | 44.55 | 83.64 | 79.5 | 67.62 | 114.66 |
| 21 | 68.62 | 50.4 | 97.44 | 76.5 | 88.2 | 156.18 |
| 25 | 80.3 | 62.1 | 107.1 | 96.8 | 108.81 | 155.1 |

2A3+ SYN-115 (100 mg/kg)

| Day | Mouse 1 | Mouse 2 | Mouse 3 | Mouse 4 | Mouse 5 | Mouse 6 |
|---|---|---|---|---|---|---|
| 0 | 12.32 | 14.82 | 15.54 | 18.33 | 21.07 | 33.64 |
| 1 | 17.76 | 17.39 | 21.42 | 15.37 | 28 | 40.47 |
| 4 | 19.6 | 16.81 | 21.5 | 19.2 | 32.4 | 50.82 |
| 6 | 23.5 | 27.03 | 30.74 | 17.5 | 33.6 | 61.6 |
| 9 | 25 | 26.52 | 47.25 | 23.1 | 40.3 | 63.19 |
| 11 | 30 | 44.25 | 58.24 | 23.4 | 56.44 | 78.26 |
| 14 | 32.4 | 38.5 | 51.94 | 29.44 | 60.2 | 88.2 |
| 16 | 44.89 | 44.85 | 69.55 | 32.5 | 63.84 | 95.4 |
| 18 | 46.08 | 61.92 | 83.95 | 40.8 | 66.3 | 103.04 |
| 21 | 50.4 | 58.8 | 99.96 | 51.8 | 76.56 | 136.08 |
| 25 | 57.96 | 54.51 | 119.04 | 50.32 | 70.4 | 135.68 |

Anti-PD-1 (200 ug)+Vehicle

| Day | Mouse 1 | Mouse 2 | Mouse 3 | Mouse 4 | Mouse 5 | Mouse 6 |
|---|---|---|---|---|---|---|
| 0 | 13.44 | 14.44 | 15.84 | 17.64 | 23.22 | 26.95 |
| 1 | 11.7 | 19.36 | 7.02 | 18.49 | 35.36 | 29.68 |
| 4 | 13.69 | 26.01 | 10.25 | 18.45 | 42.34 | 35.84 |
| 6 | 21.6 | 20.25 | 14.88 | 23.97 | 47.88 | 54.27 |
| 9 | 20.25 | 20.24 | 18 | 23.5 | 51.68 | 51.2 |
| 11 | 25.3 | 33.66 | 15.91 | 30.8 | 53.72 | 61.56 |
| 14 | 26.88 | 31.32 | 20.52 | 29.16 | 52.65 | 64.08 |
| 16 | 32.86 | 31.8 | 26.84 | 31.36 | 58.65 | 66.88 |
| 18 | 42.88 | 39.04 | 33.66 | 37.92 | 74.46 | 96.39 |
| 21 | 47.6 | 44.89 | 35 | 33.06 | 100.58 | 117.6 |
| 25 | 55.08 | 44.08 | 61.41 | 39.44 | 101.52 | 141.7 |

Anti-PD-1 (200 ug)+SYN-115 (100 mg/kg)

| Day | Mouse 1 | Mouse 2 | Mouse 3 | Mouse 4 | Mouse 5 | Mouse 6 |
|---|---|---|---|---|---|---|
| 0 | 13.76 | 14 | 17.2 | 17.49 | 26.22 | 26.4 |
| 1 | 15.75 | 24 | 19.35 | 20 | 29.61 | 39.76 |
| 4 | 17.64 | 24.4 | 16.81 | 18 | 39.69 | 40.02 |
| 6 | 16 | 23.04 | 18 | 15 | 43.4 | 41.3 |
| 9 | 18.9 | 19.8 | 11.52 | 19 | 37.8 | 37.76 |
| 11 | 15.23 | 22.05 | 8.4 | 16 | 36.48 | 40.32 |
| 14 | 15.21 | 26.52 | 11.55 | 21 | 41.48 | 42.78 |
| 16 | 16 | 34.78 | 7.5 | 20 | 49.4 | 51.2 |
| 18 | 16 | 32.43 | 0 | 20 | 44.5 | 46.4 |
| 21 | 16.8 | 45.1 | 0 | 24.2 | 47.45 | 66.6 |
| 25 | 10.15 | 47.56 | 0 | 23.65 | 67.16 | 64.24 |

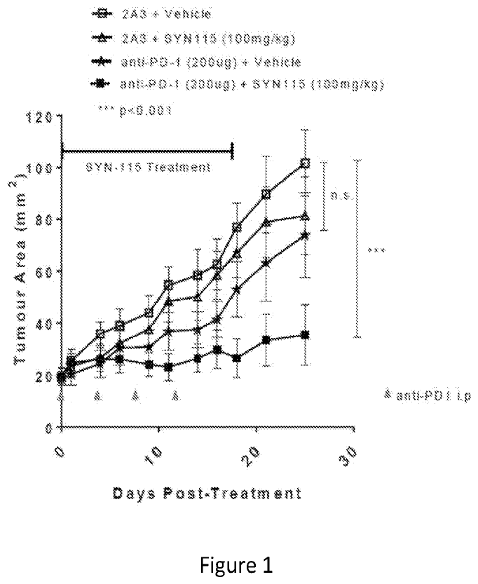

Results are shown as the mean±SEM of n=6 mice per group in FIG. 1. Statistical test was a Two-Way ANOVA/Bonferroni's multiple comparison test.

***p<0.001.

### Conclusions

SYN-115 enhanced the anti-tumor efficacy of anti-PD-1 in the AT-3 ova CD73 model.

### REFERENCES

1. Mattarollo S R, Loi S, Duret H, Ma Y, Zitvogel L, Smyth M J. Pivotal role of innate and adaptive immunity in anthracycline chemotherapy of established tumors. Cancer Res 2011; 71:4809-20. CrossRefPubMedGoogle Scholar
2. Gilfillan S, Chan C J, Cella M, Haynes N M, Rapaport A S, Boles K S, et al. DNAM-1 promotes activation of cytotoxic lymphocytes by nonprofessional antigen-presenting cells and tumors. J Exp Med 2008; 205:2965-73.
3. Beavis P A, Divisekera U, Paget C, Chow M T, John L B, Devaud C, et al. Blockade of A2A receptors potently suppresses the metastasis of CD73+ tumors. Proc Natl Acad Sci USA 2013; 110:14711-6.

### 7.2 Example 2: Evaluation of Anti-Tumor Immune Responses of SYN-115 in the AT-3ova CD73+ Tumour Cell Mouse Mmodel

### Methods:

The C57BL/6 mouse breast carcinoma cell line AT-3 was obtained from Dr. Trina Stewart (Griffith University, Nathan, QLD, Australia. Tumor cell lines were transduced to express chicken ovalbumin peptide and CD73 as previously described (1, 2, 3). Tumor cells are periodically verified to be Mycoplasma negative by the Victorian Infectious Diseases References Lab (Melbourne, VIC, Australia) by PCR analysis. Tumor cells were grown in DMEM supplemented with 10% FCS, GlutaMAX, and penicillin/streptomycin. 5×10⁵ cells were resuspended in PBS and injected s.c. into C57/BL6 mice in a 100 μL volume. At day 14 tumors were measured and mice were randomized into distinct treatment groups to ensure that each group had tumors of similar size at the onset of treatment. Mice were treated i.p. at days 14, 18, 22, and 26 with either isotype control (2A3; 200 μg/mouse) or anti-PD-1 mAb (RMP1-14, 200 μg/mouse) and where indicated, SYN-115 (Batch 71236AA002) at the indicated dose or vehicle control once-daily via oral gavage on days 14 to 29. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on rotating mixer for at least 2 hours and maintained at 4° C. Tumor measurements were taken 2-3 times per week. Tumor sizes were determined by width * length and mice were culled when tumors reached the ethical limit of 150 mm².

Results:

Raw Tumor Measurements:

2A3 (200 μg/Mouse)/Vehicle

| | Mouse | Mouse | Mouse | Mouse | Mouse | Mouse | Mouse | Mouse | Mouse |
|---|---|---|---|---|---|---|---|---|---|
| Day | 1 | 2 | 3 | 4 | 5 | 6 | 7 | 8 | 9 |
| 0 | 14.8 | 18.49 | 18.62 | 21 | 21.15 | 22.88 | 23.03 | 26.52 | 27.5 |
| 2 | 18.06 | 29.5 | 28.2 | 24.64 | 24.75 | 27.04 | 26 | 29.5 | 36.58 |
| 4 | 15.91 | 21.6 | 36.21 | 40.8 | 34.1 | 36.18 | 25.92 | 34.56 | 38.64 |
| 7 | 17.63 | 35.64 | 39.42 | 42.7 | 38.94 | 40.26 | 34.16 | 48.91 | 51.83 |
| 9 | 18.06 | 39.04 | 45.5 | 45.99 | 40.2 | 44.02 | 40.95 | 51.59 | 50.4 |
| 11 | 26.4 | 46.92 | 48.75 | 71.61 | 42.88 | 57.62 | 45.56 | 70.52 | 58.93 |
| 14 | 25.97 | 68.88 | 51.48 | 94.08 | 52.54 | 62.9 | 49.7 | 68.82 | 69.66 |
| 16 | 33.6 | 68 | 58.1 | 88.2 | 46.86 | 59.94 | 59.28 | 78.4 | 72.24 |
| 22 | 23 | 50.82 | 69.75 | 100.44 | 59.2 | 58.46 | 56.94 | 103.68 | 94.86 |
| 25 | 31.27 | 66.99 | 76.23 | 143.84 | 60.75 | 69.72 | 78.3 | 102.87 | 107.12 |
| 28 | 24.75 | 83.42 | 84.48 | | 84.48 | 74.8 | 84.48 | | 120.19 |
| 32 | 35.2 | 96.72 | 107.69 | | 106.09 | 102.9 | 110.09 | | |
| 36 | 36.04 | 114.39 | | | | | | | |
| 39 | 51 | | | | | | | | |
| 45 | 77.08 | | | | | | | | |
| 50 | 87.42 | | | | | | | | |
| 53 | 105.04 | | | | | | | | |

2A3 (200 μg/Mouse)+SYN-115 (100 mg/kg)

| | Mouse | Mouse | Mouse | Mouse | Mouse | Mouse | Mouse | Mouse | Mouse |
|---|---|---|---|---|---|---|---|---|---|
| Day | 1 | 2 | 3 | 4 | 5 | 6 | 7 | 8 | 9 |
| 0 | 14.82 | 18.49 | 19.35 | 20.72 | 21.16 | 22.56 | 24.01 | 26.52 | 27.56 |
| 2 | 30.09 | 24.01 | 24.96 | 32.64 | 29.12 | 31.35 | 27.56 | 33 | 30.8 |
| 4 | 38.35 | 24.99 | 23.37 | 35.75 | 27.5 | 46.5 | 25.2 | 46.36 | 30.16 |
| 7 | 38.28 | 29.16 | 30.5 | 38.08 | 36 | 43.4 | 44.2 | 54.4 | 33.04 |
| 9 | 37.17 | 39.65 | 40.32 | 46.8 | 46.24 | 45.5 | 51.8 | 48 | 33.63 |
| 11 | 34.1 | 40.92 | 61.92 | 63.2 | 63.08 | 55.38 | 57.72 | 50.82 | 48.75 |
| 14 | 38.43 | 40.32 | 62.37 | 59.28 | 69.72 | 60.83 | 76.36 | 60 | 46.86 |
| 16 | 51.8 | 49.68 | 59.2 | 66.4 | 80.08 | 60.8 | 83.7 | 59.94 | 52.5 |
| 22 | 55.44 | 50.41 | 34.31 | 79.98 | 89 | 38.19 | 110.16 | 56.21 | 52.44 |
| 25 | 61.77 | 77.19 | 33.6 | 88.36 | 114.13 | 59.64 | 118.17 | 54.75 | 45.44 |
| 28 | 57.75 | 79.38 | 58.28 | 144.64 | | 70.07 | | 74.52 | 63.18 |
| 32 | 89.18 | 107.06 | 54.4 | | | 99.9 | | 110.24 | 66.36 |
| 36 | 87.36 | | 48 | | | 136.74 | | | 78.96 |
| 39 | 123.2 | | 91.35 | | | | | | 103.88 |
| 45 | | | 143 | | | | | | |

Anti-PD-1 (200 ug)+Vehicle

| | Mouse | Mouse | Mouse | Mouse | Mouse | Mouse | Mouse | Mouse | Mouse |
|---|---|---|---|---|---|---|---|---|---|
| Day | 1 | 2 | 3 | 4 | 5 | 6 | 7 | 8 | 9 |
| 0 | 14.82 | 18.49 | 19.35 | 20.72 | 21.16 | 22.56 | 24.01 | 26.52 | 27.56 |
| 2 | 30.09 | 24.01 | 24.96 | 32.64 | 29.12 | 31.35 | 27.56 | 33 | 30.8 |
| 4 | 38.35 | 24.99 | 23.37 | 35.75 | 27.5 | 46.5 | 25.2 | 46.36 | 30.16 |
| 7 | 38.28 | 29.16 | 30.5 | 38.08 | 36 | 43.4 | 44.2 | 54.4 | 33.04 |
| 9 | 37.17 | 39.65 | 40.32 | 46.8 | 46.24 | 45.5 | 51.8 | 48 | 33.63 |
| 11 | 34.1 | 40.92 | 61.92 | 63.2 | 63.08 | 55.38 | 57.72 | 50.82 | 48.75 |
| 14 | 38.43 | 40.32 | 62.37 | 59.28 | 69.72 | 60.83 | 76.36 | 60 | 46.86 |
| 16 | 51.8 | 49.68 | 59.2 | 66.4 | 80.08 | 60.8 | 83.7 | 59.94 | 52.5 |
| 22 | 55.44 | 50.41 | 34.31 | 79.98 | 89 | 38.19 | 110.16 | 56.21 | 52.44 |
| 25 | 61.77 | 77.19 | 33.6 | 88.36 | 114.13 | 59.64 | 118.17 | 54.75 | 45.44 |
| 28 | 57.75 | 79.38 | 58.28 | 144.64 | | 70.07 | | 74.52 | 63.18 |
| 32 | 89.18 | 107.06 | 54.4 | | | 99.9 | | 110.24 | 66.36 |
| 36 | 87.36 | | 48 | | | 136.74 | | | 78.96 |
| 39 | 123.2 | | 91.35 | | | | | | 103.88 |
| 45 | | | 143 | | | | | | |

Anti-PD-1 (200 ug)+SYN-115 (10 mg/kg)

| | Mouse | Mouse | Mouse | Mouse | Mouse | Mouse | Mouse | Mouse | Mouse | Mouse |
|---|---|---|---|---|---|---|---|---|---|---|
| Day | 1 | 2 | 3 | 4 | 5 | 6 | 7 | 8 | 9 | 10 |
| 0 | 15.2 | 17.22 | 19.36 | 20.24 | 21.62 | 22.5 | 24.75 | 26.52 | 30.8 | 32.94 |
| 2 | 14.72 | 14.8 | 26.52 | 21.16 | 31.72 | 24.96 | 31.86 | 28.09 | 31.62 | 35.91 |
| 4 | 17.86 | 14 | 25.85 | 13.94 | 32.56 | 23.37 | 43.07 | 26.55 | 44.82 | 33.55 |
| 7 | 13.69 | 18.48 | 29.16 | 23.04 | 32 | 30.74 | 47.6 | 40.87 | 45.14 | 38.44 |
| 9 | 15.2 | 23.04 | 36.48 | 25.48 | 35.34 | 31.62 | 51.1 | 42.7 | 46.9 | 35.84 |
| 11 | 17.6 | 27.5 | 42.16 | 14.04 | 45.75 | 52.46 | 48.84 | 41.4 | 45.75 | 29.68 |
| 14 | 22.08 | 29.68 | 51.12 | 28.08 | 38.5 | 50.56 | 58.93 | 78.96 | 55.25 | 43.31 |
| 16 | 21 | 38.4 | 57.75 | 36 | 45.14 | 48 | 61.42 | 79.9 | 54.56 | 48.1 |
| 22 | 17.6 | 38.19 | 66.12 | 31.92 | 39.42 | 50.05 | 79.12 | 100.62 | 67.68 | 53.04 |
| 25 | 25.92 | 39.04 | 80.08 | 31.27 | 41.3 | 58.48 | 98 | 136.22 | 83.64 | 63.51 |
| 28 | 20.64 | 58.52 | 86.45 | 38.28 | 49 | 69.42 | 119.6 | | 111.6 | 74.8 |
| 32 | 23.5 | 60.04 | 118.32 | 34.22 | 64.24 | 91.18 | | | | 92.82 |
| 36 | 30.68 | 73.08 | | 61.32 | 89 | 114.13 | | | | 124 |
| 39 | 25.92 | 86.24 | | 55.89 | 133.44 | | | | | |
| 45 | 39.65 | 126.5 | | 65.45 | | | | | | |
| 50 | 48.38 | | | 66.22 | | | | | | |
| 53 | 53.6 | | | 83.72 | | | | | | |
| 57 | 63.19 | | | 81.6 | | | | | | |
| 60 | 72.72 | | | 80.04 | | | | | | |
| 64 | 91.64 | | | 104.86 | | | | | | |
| 68 | 121.26 | | | | | | | | | |

Anti-PD-1 (200 ug)+SYN-115 (30 mg/kg)

| | Mouse | Mouse | Mouse | Mouse | Mouse | Mouse | Mouse | Mouse | Mouse | Mouse |
|---|---|---|---|---|---|---|---|---|---|---|
| Day | 1 | 2 | 3 | 4 | 5 | 6 | 7 | 8 | 9 | 10 |
| 0 | 15.21 | 15.96 | 19.78 | 20 | 21.93 | 22.36 | 24.99 | 26 | 31.35 | 32.49 |
| 2 | 21.16 | 14.06 | 15.3 | 18.24 | 28.09 | 26.01 | 20.25 | 19.36 | 44.85 | 32.4 |
| 4 | 14.4 | 11.4 | 12.54 | 16.77 | 21.12 | 26.22 | 16.92 | 26.68 | 28.6 | 27.52 |
| 7 | 19.36 | 17.64 | 16.8 | 18.62 | 29.28 | 30.87 | 27.03 | 20.7 | 36.4 | 33.15 |
| 9 | 24.96 | 18.92 | 18.49 | 22.14 | 28.08 | 38.28 | 31.5 | 23.04 | 42.25 | 39 |
| 11 | 24.44 | 16.56 | 14.52 | 13.44 | 25.85 | 33.63 | 35.75 | 16.77 | 36.6 | 48 |
| 14 | 44.73 | 22.08 | 11.88 | 32 | 30.08 | 43.52 | 43.47 | 24.01 | 58.46 | 44.8 |
| 16 | 57 | 24.5 | 16.81 | 29.44 | 38.4 | 48.3 | 45.5 | 31.35 | 62.41 | 37.7 |
| 22 | 61.32 | 17.94 | 17.63 | 38.16 | 32.83 | 50.25 | 31.92 | 30.68 | 62.78 | 31.85 |
| 25 | 75.68 | 23.5 | 11.96 | 45.03 | 40.26 | 52.65 | 39 | 33.48 | 76.95 | 35.2 |
| 28 | 100.88 | 23.03 | 22.09 | 48.98 | 38.88 | 70.08 | 39.6 | 36.58 | 72 | 43.92 |
| 32 | 111.72 | 24.38 | 24.91 | 53.13 | 54.81 | 83.43 | 56.24 | 36.48 | 78.72 | 41.54 |
| 36 | | 29.15 | 29.07 | 43.68 | 63.24 | 106.72 | 70.47 | 39.65 | 113.4 | 41.48 |
| 39 | | 26.23 | 28.98 | 90.24 | 69 | | 65.96 | 50.05 | | 54.18 |
| 45 | | 45.26 | 34 | | 97.11 | | 117.12 | 73.1 | | 73.92 |
| 50 | | 63.9 | 32.86 | | 99.45 | | | 88.2 | | 96.72 |
| 53 | | 59.04 | 46.97 | | 100.1 | | | 106.05 | | 111.36 |
| 57 | | 75.24 | 44.22 | | | | | | | |
| 60 | | 89.25 | 48.28 | | | | | | | |
| 64 | | 107.1 | 69.52 | | | | | | | |
| 68 | | | 92.22 | | | | | | | |

Anti-PD-1 (200 ug)+SYN-115 (100 mg/kg)

| | Mouse | Mouse | Mouse | Mouse | Mouse | Mouse | Mouse | Mouse | Mouse | Mouse |
|---|---|---|---|---|---|---|---|---|---|---|
| Day | 1 | 2 | 3 | 4 | 5 | 6 | 7 | 8 | 9 | 10 |
| 0 | 15.6 | 15.6 | 19.8 | 19.8 | 22.05 | 22.09 | 25 | 25.76 | 31.5 | 31.92 |
| 2 | 15.21 | 23.52 | 17.39 | 21.16 | 28.5 | 32.45 | 28.09 | 25.2 | 31.5 | 24 |
| 4 | 21.93 | 18.48 | 16.77 | 21.6 | 26.95 | 28.91 | 26.52 | 23.5 | 39.44 | 20.68 |
| 7 | 16.81 | 25 | 17.2 | 18.92 | 32.83 | 34.2 | 25.92 | 29.89 | 26.32 | 27.04 |
| 9 | 14.06 | 24.91 | 18.06 | 20.7 | 29.28 | 41.6 | 33.55 | 27.36 | 25.85 | 25.97 |
| 11 | 14.35 | 20.91 | 18.06 | 18.4 | 30.55 | 37.26 | 31.9 | 23.22 | 22 | 31.35 |
| 14 | 22.05 | 26.79 | 23.03 | 20.7 | 57.75 | 50.25 | 39.68 | 34.02 | 30.6 | 44.2 |
| 16 | 28.5 | 30.5 | 27.54 | 23.5 | 48.8 | 48 | 46.92 | 40.8 | 35.2 | 51.1 |
| 22 | 22.05 | 46.98 | 27 | 26.22 | 61.5 | 46.92 | 57.6 | 47.36 | 39.04 | 56.7 |
| 25 | 26.95 | 56.28 | 34.72 | 36.58 | 78.12 | 60.83 | 82.56 | 65.55 | 44.22 | 67.23 |
| 28 | 38.28 | 71.78 | 43.8 | 31.86 | 84.63 | 64.38 | 74.76 | 72.27 | 68.82 | 80.84 |
| 32 | 39.76 | 75.33 | 60.59 | 44.8 | 103 | 83.3 | 80.64 | 78.12 | 85.85 | 97 |
| 36 | 42.9 | 92.4 | 59.29 | 45.5 | | 104.34 | 117.66 | 93.45 | 92.22 | 105.04 |
| 39 | 66 | 119.38 | 79.9 | 62.48 | | | | 139.52 | 114.24 | |
| 45 | 101 | | 129.71 | 75.84 | | | | | | |
| 50 | | | | 136.4 | | | | | | |

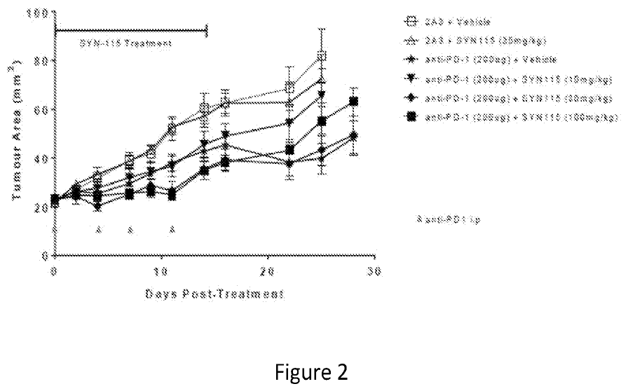

Results are shown as the mean±SEM of n=9-10 mice per group in FIG. 2.

### Conclusions

Although SYN-115 appeared to enhance the anti-tumor efficacy of anti-PD-1 at early timepoints (Days 0-14), this effect was not sustained. Most notable was the reduction in tumor size in the anti-PD-1 only/vehicle group at days 15-21 which was out of keeping with the previous experiments in this model including Example 1.

### REFERENCES

1. Mattarollo S R, Loi S, Duret H, Ma Y, Zitvogel L, Smyth M J. Pivotal role of innate and adaptive immunity in anthracycline chemotherapy of established tumors. Cancer Res 2011; 71:4809-20. CrossRefPubMedGoogle Scholar
2. Gilfillan S, Chan C J, Cella M, Haynes N M, Rapaport A S, Boles K S, et al. DNAM-1 promotes activation of cytotoxic lymphocytes by nonprofessional antigen-presenting cells and tumors. J Exp Med 2008; 205:2965-73.
3. Beavis P A, Divisekera U, Paget C, Chow M T, John L B, Devaud C, et al. Blockade of A2A receptors potently suppresses the metastasis of CD73+ tumors. Proc Natl Acad Sci U.S.A. 2013;110:14711-6.

### 7.3 Example 3: Evaluation of Anti-Tumor Immune Responses of SYN-115 in the AT-3ova CD73+ Tumour Cell Mouse Model

### Methods:

The C57BL/6 mouse breast carcinoma cell line AT-3 was obtained from Dr. Trina Stewart (Griffith University, Nathan, QLD, Australia. Tumor cell lines were transduced to express chicken ovalbumin peptide and CD73 as previously described (1, 2, 3). Tumor cells are periodically verified to be Mycoplasma negative by the Victorian Infectious Diseases References Lab (Melbourne, VIC, Australia) by PCR analysis. Tumor cells were grown in DMEM supplemented with 10% FCS, GlutaMAX, and penicillin/streptomycin. 5×10⁵ cells were resuspended in PBS and injected s.c. into C57/BL6 mice in a 100 μL volume. At day 14 tumors were measured and mice were randomized into distinct treatment groups to ensure that each group had tumors of similar size at the onset of treatment. Mice were treated i.p. at days 14, 18, 22, and 26 with either isotype control (2A3; 200 μg/mouse) or anti-PD-1 mAb (RMP1-14, 200 μg/mouse) and where indicated SYN-115 (Batch 71236AA002) at the indicated dose or vehicle control once or twice-daily via oral gavage on days 14 to 29. To calculate a dose of SYN-115 mouse weight was estimated to be 20 g per mouse. Therefore, 100 mg/kg=2 mg per mouse in 200 which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

### Results:

Raw Tumor Measurements:

2A3 (200 μg/Mouse)/Vehicle QD

| Day | Mouse | Mouse 2 | Mouse 3 | Mouse 4 | Mouse 5 | Mouse |
|---|---|---|---|---|---|---|
| 0 | 20.72 | 11.4 | 21.66 | 10.23 | 18.33 | 20.68 |
| 3 | 34.02 | 12.95 | 28.29 | 14.35 | 27.28 | 25.52 |
| 6 | 38.34 | 15.58 | 35.69 | 21.6 | 31.32 | 45.24 |
| 9 | 50.15 | 18.33 | 53.1 | 34.22 | 46.15 | 57.96 |
| 13 | 75.46 | 27.54 | 60 | 37.17 | 68.06 | 61.32 |
| 15 | 80.34 | 27.54 | 64.26 | 39.68 | 67.2 | 76.26 |
| 17 | 92.96 | 29.68 | 85.12 | 57.27 | 87 | 70.07 |
| 20 | 113.03 | 29.07 | 104.92 | 67.15 | 114.66 | 93.84 |
| 22 | 116.25 | 39.6 | 127.53 | 68.04 | 108.58 | 131.76 |
| 24 | | 44.53 | | 96 | | |
| 28 | | 60 | | 102.72 | | |
| 31 | | 63.08 | | | | |
| 34 | | 66.12 | | | | |
| 39 | | 82.8 | | | | |
| 42 | | 104 | | | | |

Anti-PD-1 (200 ug)+Vehicle QD

| Day | Mouse 1 | Mouse 2 | Mouse 3 | Mouse 4 | Mouse 5 | Mouse 6 |
|---|---|---|---|---|---|---|
| 0 | 19.44 | 12.16 | 14.06 | 23.01 | 23.92 | 18 |
| 3 | 25.74 | 15.6 | 18.48 | 30.38 | 25.48 | 19.27 |
| 6 | 34.32 | 27.14 | 25.08 | 44.4 | 22.88 | 25.65 |
| 9 | 36 | 23.5 | 33.6 | 49.6 | 25.2 | 34.72 |
| 13 | 49.5 | 23.97 | 36.54 | 54.78 | 19.78 | 33.48 |
| 15 | 59.92 | 31.35 | 46.97 | 63.7 | 22.08 | 41.3 |
| 17 | 73.6 | 30.21 | 62.56 | 67.5 | 27.54 | 39.33 |
| 20 | 97.29 | 38.4 | 71.61 | 73.15 | 43.07 | 60.84 |
| 22 | 111 | 36.54 | 82.95 | 76.22 | 36.6 | 45.26 |
| 24 | | 40.8 | 71.44 | 82.62 | 35.34 | 64.5 |
| 28 | | 46.9 | 102.5 | 112.53 | 60.8 | 80 |
| 31 | | 54 | | | 71.4 | 80 |
| 34 | | 71.44 | | | 72.8 | 110.4 |
| 39 | | 92.02 | | | 96.82 | |
| 42 | | 111.3 | | | 118.32 | |

Anti-PD-1 (200 ug)+SYN-115 (30 mg/kg) QD

| Day | Mouse 1 | Mouse 2 | Mouse 3 | Mouse 4 | Mouse 5 | Mouse 6 |
|---|---|---|---|---|---|---|
| 0 | 5.98 | 21 | 18.24 | 14.28 | 19.8 | 20.09 |
| 3 | 12.21 | 25.85 | 24.08 | 16.28 | 23.4 | 22 |
| 6 | 13.2 | 33.39 | 34.32 | 22.5 | 31.02 | 39.9 |
| 9 | 13.8 | 41.3 | 42.88 | 28.09 | 39.2 | 40.8 |
| 13 | 24.84 | 49.28 | 48.19 | 27 | 43.45 | 54.78 |
| 15 | 29.9 | 50.4 | 64.08 | 29.64 | 51.3 | 76.8 |
| 17 | 28.6 | 57.67 | 64.97 | 37.05 | 56.42 | 66.56 |
| 20 | 33.64 | 70.56 | 75.53 | 40.2 | 87.69 | 82.32 |
| 22 | 46.02 | 80.84 | 71.2 | 40.26 | 76.68 | 87.98 |
| 24 | 63.7 | 102.72 | 102.9 | 42.25 | 92.66 | 118.58 |
| 28 | 58.5 | | | 63.99 | 113.1 | |
| 31 | 57.76 | | | 74.82 | | |
| 34 | 72.8 | | | 93.06 | | |
| 39 | 83.42 | | | 127 | | |
| 42 | 127.53 | | | | | |

Anti-PD-1 (200 ug)+SYN-115 (30 mg/kg) BID

| Day | Mouse 1 | Mouse 2 | Mouse 3 | Mouse 4 | Mouse 5 | Mouse 6 |
|---|---|---|---|---|---|---|
| 0 | 12.6 | 15.54 | 22 | 16.38 | 22.68 | 18.36 |
| 3 | 15.84 | 21.15 | 21.2 | 16.38 | 37.95 | 31.49 |
| 6 | 21.45 | 29.7 | 35.2 | 16.28 | 44.25 | 32.5 |
| 9 | 21.35 | 31.35 | 43.07 | 16.8 | 45.6 | 46.62 |
| 13 | 36.57 | 34.1 | 53.76 | 23.03 | 52.8 | 53.12 |
| 15 | 38.64 | 39.33 | 53.6 | 16.72 | 68.64 | 50.4 |
| 17 | 44.08 | 44.22 | 73.92 | 38.86 | 63.24 | 66.64 |
| 20 | 63.9 | 52.54 | 85.14 | 27.44 | 81.7 | 76.14 |
| 22 | 61.2 | 58.4 | 99.84 | 45.5 | 89.76 | 73.5 |
| 24 | 88.4 | 72.16 | 116.56 | 34.2 | 77.08 | 94.34 |
| 28 | 113.28 | 94.05 | | 46.62 | 105.45 | 122.2 |
| 31 | | 111.28 | | 64.74 | | |
| 34 | | | | 73.1 | | |
| 39 | | | | 69.6 | | |
| 42 | | | | 99.51 | | |
| 44 | | | | 114.33 | | |

2A3 (200 ug/mouse)+SYN-115 (100 mg/kg) QD

| Day | Mouse | Mouse 2 | Mouse 3 | Mouse 4 | Mouse 5 | Mouse 6 |
|---|---|---|---|---|---|---|
| 0 | 19.38 | 14.96 | 20 | 24.38 | 13.26 | 18.9 |
| 3 | 28.35 | 18.9 | 22.54 | 32.94 | 14.06 | 22.4 |
| 6 | 34.45 | 31.62 | 24.19 | 41.3 | 14.82 | 29.04 |
| 9 | 43.31 | 38.76 | 39.6 | 54.4 | 18.48 | 35.36 |
| 13 | 61.88 | 46.48 | 54.94 | 58.8 | 23.52 | 36.48 |
| 15 | 66.24 | 52.56 | 60.2 | 73.44 | 31.32 | 50.84 |
| 17 | 69.92 | 62.32 | 68.4 | 80.36 | 31.9 | 49.56 |
| 20 | 115.2 | 82.32 | 86.7 | 98.28 | 34.81 | 69 |
| 22 | 107.01 | 89.61 | 85.68 | 99.12 | 43.92 | 70.29 |
| 24 | | 109.04 | 104.4 | 122.85 | 47.88 | 82.68 |
| 28 | | | | | 48.96 | 108.56 |
| 31 | | | | | 73.1 | |
| 34 | | | | | 74.82 | |
| 39 | | | | | 86.45 | |
| 42 | | | | | 129.87 | |

Anti-PD-1 (200 ug)+SYN-115 (100 mg/kg) QD

| Day | Mouse 1 | Mouse 2 | Mouse 3 | Mouse 4 | Mouse 5 | Mouse 6 |
|---|---|---|---|---|---|---|
| 0 | 14.82 | 14.76 | 15.17 | 15.12 | 14.82 | 23.78 |
| 3 | 23.65 | 19.35 | 12.4 | 22.56 | 24.91 | 26.66 |
| 6 | 34.32 | 23.92 | 16.1 | 24.44 | 28.4 | 18.92 |
| 9 | 29.07 | 25 | 29.68 | 26 | 34.78 | 29.25 |
| 13 | 32.45 | 39.33 | 38.19 | 25.48 | 40.7 | 33.5 |
| 15 | 39.33 | 42 | 44.2 | 32.86 | 50.74 | 25.85 |
| 17 | 39.53 | 44.4 | 48 | 31.35 | 52.92 | 44.73 |
| 20 | 48.28 | 61.56 | 66.6 | 48.99 | 61.2 | 51.2 |
| 22 | 51.1 | 57.67 | 77.22 | 37.82 | 67.45 | 38.5 |
| 24 | 51.83 | 72.98 | 86.92 | 53.96 | 85.05 | 46.8 |
| 28 | 55.2 | 90.09 | 97.9 | 77.44 | 95.04 | 77.19 |
| 31 | 74.82 | | 128.76 | 81.7 | 101.37 | 79.9 |
| 34 | 79.05 | | | 95.06 | | 81.32 |
| 39 | 81.9 | | | 104.86 | | 82.95 |
| 42 | 127.33 | | | | | 132.24 |

Anti-PD-1 (200 ug)+SYN-115 (100 mg/kg) BID

| Day | Mouse 1 | Mouse 2 | Mouse 3 | Mouse 4 | Mouse 5 | Mouse |
|---|---|---|---|---|---|---|
| 0 | 16.28 | 15.96 | 20.46 | 16.8 | 24.12 | 12.24 |
| 3 | 21.15 | 23.32 | 19.32 | 20.28 | 26.8 | 23.03 |
| 6 | 27.93 | 25.52 | 36.92 | 24.96 | 40.04 | 30.25 |
| 9 | 32.33 | 36.72 | 38.4 | 35.88 | 43.46 | 41.04 |
| 13 | 31.2 | 45.9 | 52.8 | 43.32 | 41.86 | 43.12 |
| 15 | 39.76 | 43.52 | 52.14 | 41.76 | 50.88 | 63.36 |
| 17 | 36.18 | 87.42 | 65.36 | 43.5 | 63.24 | 67.16 |
| 20 | 48 | 94.34 | 74 | 55.44 | 77.05 | 77.22 |
| 22 | 49.92 | 83.7 | 67.45 | 51.48 | 75.64 | 82.39 |
| 24 | 53.04 | 108.07 | 75.24 | 77.76 | 83.08 | 99.76 |
| 28 | 66.96 | | 98.79 | 88.4 | 132.3 | 118.34 |
| 31 | 79.38 | | 102.08 | 109.44 | | |
| 34 | 99.36 | | | | | |
| 39 | 103.55 | | | | | |
| 42 | | | | | | |

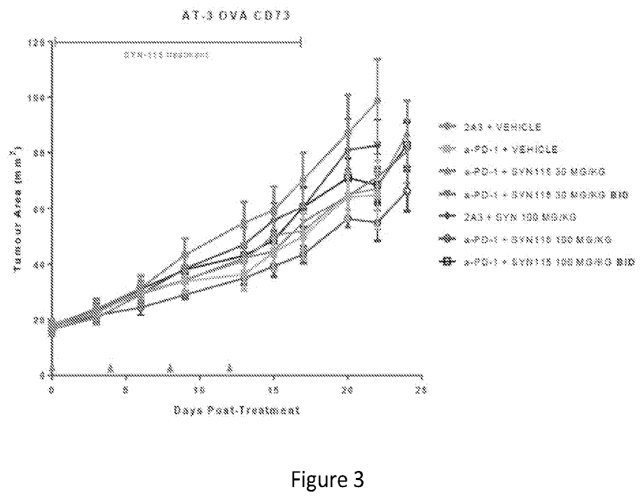

Results are shown as the mean±SEM of n=6 mice per group in FIG. 3.

### Conclusions

In this experiment, no anti-tumor activity was observed with SYN-115 at either 30 mg/kg or 100 mg/kg either alone or in combination with anti-PD-1. Due to the lack of efficacy observed in Example 3 and Example 9, a new batch of SYN-115 was obtained. The new batch was used in Examples 10, 11, 12 and 13.

### REFERENCES

1. Mattarollo S R, Loi S, Duret H, Ma Y, Zitvogel L, Smyth M J. Pivotal role of innate and adaptive immunity in anthracycline chemotherapy of established tumors. Cancer Res 2011; 71:4809-20. CrossRefPubMedGoogle Scholar
2. Gilfillan S, Chan C J, Cella M, Haynes N M, Rapaport A S, Boles K S, et al. DNAM-1 promotes activation of cytotoxic lymphocytes by nonprofessional antigen-presenting cells and tumors. J Exp Med 2008; 205:2965-73.
3. Beavis P A, Divisekera U, Paget C, Chow M T, John L B, Devaud C, et al. Blockade of A2A receptors potently suppresses the metastasis of CD73+ tumors. Proc Natl Acad Sci U.S.A. 2013; 110:14711-6.

### 7.4 Example 4: Evaluation of Anti-Tumor Immune Responses of SYN-115 in the CT26 Tumor Mouse Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×10⁵ cells were resuspended in PBS and injected s.c. into BALB/c mice in a 100 μL volume. At day 9, tumors were measured and mice were randomized into distinct treatment groups to ensure that each group had tumors of similar size at the onset of treatment. Mice were treated i.p. at days 9, 13 and 17 with either isotype control (2A3; 200 μg/mouse) or anti-PD-1 mAb (RMP1-14, 200 μg/mouse) and where indicated, SYN-115 (Batch 71236AA002) at the indicated dose or vehicle control once-daily via oral gavage on days 14 to 29. To calculate a dose of SYN-115 mouse weight was estimated to be 20 g per mouse. Therefore, 100 mg/kg=2 mg per mouse in 200 which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

Results:

Raw Tumor Measurements:

2A3 (200 μg/mouse)/Vehicle

| Day | Mouse 1 | Mouse 2 | Mouse 3 | Mouse 4 | Mouse 5 | Mouse 6 |
|---|---|---|---|---|---|---|
| 0 | 12.21 | 14.96 | 15.48 | 19.2 | 19.38 | 22.68 |
| 4 | 34.72 | 43.07 | 37.44 | 42.93 | 47.88 | 57.85 |
| 6 | 66.43 | 57.12 | 50.41 | 61.88 | 76.95 | 74.46 |
| 8 | 84.6 | 92.92 | 73 | 87.98 | 87.87 | 99.45 |
| 11 | 123.2 | 101.52 | 112.86 | 138.43 | 119.9 | 143.91 |
| 13 | 151.13 | 174.2 | 155.68 | 198 | 165.1 | 148.68 |
| 15 | | | | | | 200.22 |

2A3 (200 μg/mouse)+SYN-115 (30 mg/kg)

| Day | Mouse 1 | Mouse 2 | Mouse 3 | Mouse 4 | Mouse 5 | Mouse 6 |
|---|---|---|---|---|---|---|
| 0 | 12.71 | 14.85 | 16.32 | 18.92 | 20.16 | 22.36 |
| 4 | 37.62 | 42.12 | 40.7 | 53.2 | 38.4 | 52.56 |
| 6 | 43.52 | 64.6 | 51.8 | 79 | 72.9 | 90.21 |
| 8 | 61.6 | 83.93 | 59.13 | 106.92 | 100.28 | 127.2 |
| 11 | 108.64 | 116.39 | 93.1 | 198.32 | 148.48 | 198.32 |
| 13 | 146.41 | 150.7 | 93.93 | | 186.83 | |
| 15 | 188.94 | | 100.8 | | | |
| 19 | | | 186.12 | | | |

2A3 (200 μg/mouse)+SYN-115 (100 mg/kg)

| Day | Mouse 1 | Mouse 2 | Mouse 3 | Mouse 4 | Mouse 5 | Mouse 6 |
|---|---|---|---|---|---|---|
| 0 | 12.76 | 14.8 | 17 | 18.9 | 20.5 | 21.12 |
| 4 | 44.73 | 41.31 | 42.7 | 41.76 | 49.64 | 25.85 |
| 6 | 53.4 | 59.78 | 73.04 | 58.32 | 71.76 | 42.92 |
| 8 | 66.75 | 90.2 | 89 | 87.4 | 94.08 | 51.84 |
| 11 | 110.88 | 114.33 | 148.8 | 126.14 | 110 | 74.1 |
| 13 | 187.53 | 175.56 | 213.12 | 203.68 | 164.56 | 110.4 |
| 15 | | | | | | 151.42 |

Anti-PD-1 (200 ug)+Vehicle

| Day | Mouse 1 | Mouse 2 | Mouse 3 | Mouse 4 | Mouse 5 | Mouse 6 |
|---|---|---|---|---|---|---|
| 0 | 12.87 | 14.7 | 18.02 | 18.9 | 20.58 | 21.07 |
| 4 | 26.91 | 42.88 | 40.32 | 39.76 | 33.39 | 48.64 |
| 6 | 36.72 | 48.3 | 48.6 | 62.9 | 56.94 | 75 |
| 8 | 46.92 | 54.4 | 63.51 | 70.31 | 79.54 | 108 |
| 11 | 71.89 | 59.94 | 97.68 | 100.98 | 131.89 | 135.52 |
| 13 | 100.58 | 66.42 | 117.81 | 136.4 | 180.88 | 189.63 |
| 15 | 126.69 | 71.2 | 151.8 | 174.66 | | |
| 19 | 193.7 | 57.27 | | | | |
| 22 | | 70.31 | | | | |
| 25 | | 141.45 | | | | |
| 28 | | 178.22 | | | | |

Anti-PD-1 (200 ug)+SYN-115 (30 mg/kg)

| Day | Mouse 1 | Mouse 2 | Mouse 3 | Mouse 4 | Mouse 5 | Mouse 6 |
|---|---|---|---|---|---|---|
| 0 | 12.95 | 14.44 | 18.36 | 18.62 | 20.72 | 21.07 |
| 4 | 27.45 | 34.72 | 42.88 | 40.71 | 53.4 | 41.89 |
| 6 | 34.45 | 70.31 | 73.92 | 46.2 | 110.36 | 61.5 |
| 8 | 28.62 | 60.06 | 62.37 | 54.67 | 141.12 | 85 |
| 11 | 38.28 | 121.52 | 77.9 | 69.52 | 184.86 | 127.44 |
| 13 | 41.8 | 157.29 | 79.38 | 74.48 | | 207.32 |
| 15 | 44.16 | | 91.53 | 72.21 | | |
| 19 | 72.24 | | 98.79 | 101.76 | | |
| 22 | 108 | | 136.64 | 120.64 | | |
| 25 | 160.95 | | 151.2 | 159.6 | | |

Anti-PD-1 (200 ug)+SYN-115 (100 mg/kg)

| Day | Mouse 1 | Mouse 2 | Mouse 3 | Mouse 4 | Mouse 5 | Mouse 6 |
|---|---|---|---|---|---|---|
| 0 | 13.02 | 13.12 | 18.4 | 18.48 | 20.79 | 21 |
| 4 | 40.29 | 16.56 | 45.44 | 40.87 | 42.12 | 72.36 |
| 6 | 64.32 | 12.95 | 64.6 | 53.25 | 57.85 | 111.35 |
| 8 | 87.87 | 15.91 | 83.72 | 87.4 | 80.36 | 138.16 |
| 11 | 127.5 | 10.85 | 131.44 | 119.9 | 126.5 | 192.24 |
| 13 | 201.15 | 9.8 | 205.9 | 201.55 | 208.8 | |
| 15 | | 7.25 | | | | |
| 19 | | 0 | | | | |
| 22 | | 0 | | | | |
| 25 | | 0 | | | | |
| 28 | | 0 | | | | |

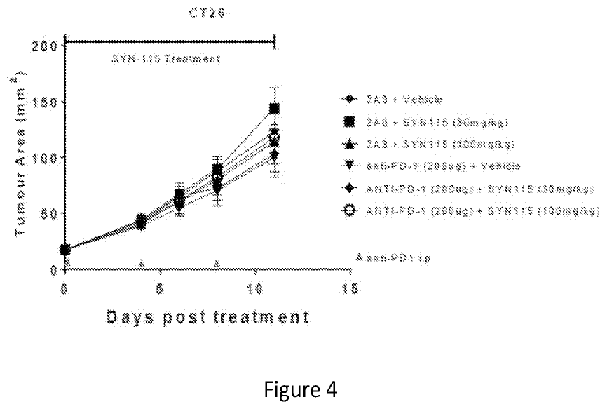

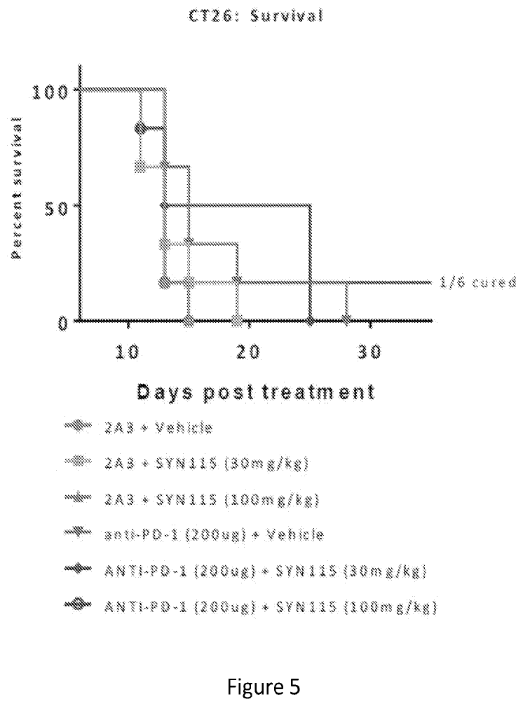

Results are shown as the mean±SEM of n=6 mice per group in FIGS. 4 and 5.

### Conclusions

Although the anti-tumor effects were not as great as expected in comparison to published work on this model where activity of A₂ₐ antagonists has been observed (1). This may be due to the large size of the tumors treated. Notably, anti-PD-1 was also ineffective in this model.

### REFERENCES

1. Willingham S B, Ho P Y, Hotson A, Hill C M, Piccione E C, Hsieh J, Liu L, Buggy J J, McCaffery I, Miller R A. A2AR Antagonism with CPI-444 Induces Anti-Tumor Responses and Augments Efficacy to Anti-PD-(L)1 and Anti-CTLA-4 in Preclinical Models. Cancer Immunol. Res. 2018.

### 7.5 Example 5: Evaluation of Anti-Tumor Immune Responses of SYN-115 in the CT26 Tumor Mouse Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×10⁵ cells were resuspended in PBS and injected s.c. into BALB/c mice in a 100 μL volume. At day 7 tumors were measured and mice were randomized into distinct treatment groups to ensure that each group had tumors of similar size at the onset of treatment. Mice were treated i.p. at days 7, 11 and 15, with either isotype control (2A3; 200 μg/mouse) or anti-PD-1 mAb (RMP1-14, 200 μg/mouse) and where indicated, SYN-115 (Batch 71236AA002) at the indicated dose or vehicle control once-daily via oral gavage on days 7 to 20. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

Results:

Raw Tumor Measurements:

2A3 (200 μg/Mouse)/Vehicle

| Day | Mouse 1 | Mouse 2 | Mouse 3 | Mouse 4 | Mouse 5 | Mouse 6 |
|---|---|---|---|---|---|---|
| 0 | 6.48 | 11 | 11.84 | 11.34 | 9.1 | 16.64 |
| 4 | 11.44 | 17.6 | 19.25 | 16.38 | 9.6 | 22.2 |
| 6 | 10.14 | 32.85 | 34.31 | 25.2 | 11.56 | 27.47 |
| 8 | 19.44 | 46.98 | 48.19 | 26.84 | 15.36 | 36.75 |
| 11 | 18.56 | 59.78 | 66.93 | 34.17 | 12.25 | 39.1 |
| 13 | 27.06 | 79.2 | 105.78 | 57.51 | 10.4 | 55.44 |
| 15 | 32.9 | 115.56 | 123.71 | 78.21 | 16.56 | 68.25 |
| 18 | 45.65 | 163.68 | 189.75 | 140.97 | 17.76 | 91.64 |
| 20 | 61 | | | 175.45 | 28.5 | 120.4 |
| 21 | | | | | | 133.86 |
| 22 | 78.81 | | | | 31.9 | 146.45 |
| 25 | 113.46 | | | | 54.72 | |
| 28 | 130.81 | | | | 87.22 | |
| 32 | 194.18 | | | | 111.28 | |
| 34 | | | | | 156.16 | |

2A3 (200 μg/Mouse)+SYN-115 (30 mg/kg)

| Day | Mouse 1 | Mouse 2 | Mouse 3 | Mouse 4 | Mouse 5 | Mouse |
|---|---|---|---|---|---|---|
| 0 | 7.82 | 12.24 | 13.63 | 7.59 | 9.18 | |
| 4 | 17.67 | 9.45 | 7.92 | 6.24 | 14.5 | |
| 6 | 21.45 | 6.48 | 6.72 | 7.29 | 9.3 | |
| 8 | 28.35 | 9.6 | 7.5 | 9.3 | 10.85 | |
| 11 | 27.9 | 8.1 | 5.94 | 3.96 | 14.4 | |
| 13 | 41.18 | 7.02 | 5.72 | 5.72 | 19.35 | |
| 15 | 56.55 | 4.62 | 5.72 | 8.7 | 23.03 | |
| 18 | 92.34 | 0 | 5.94 | 14.4 | 31.2 | |
| 20 | 120.65 | 0 | 0 | 17.1 | 37.8 | |
| 21 | 135.45 | 0 | 0 | | | |
| 22 | 140.08 | 0 | 0 | 19.27 | 53.82 | |
| 25 | 151.42 | 0 | 5.5 | 24.84 | 78.72 | |
| 28 | | 0 | 9.18 | 22.4 | 80.34 | |
| 32 | | 0 | 12.54 | 22.79 | 109.48 | |
| 34 | | 0 | 17.48 | 16.8 | 144.64 | |
| 39 | | 0 | 10.56 | 20.64 | | |
| 41 | | 0 | 9.3 | 17.16 | | |

2A3 (200 μg/Mouse)+SYN-115 (100 mg/kg)

| Day | Mouse 1 | Mouse 2 | Mouse 3 | Mouse 4 | Mouse 5 | Mouse 6 | Mouse 7 |
|---|---|---|---|---|---|---|---|
| 0 | 10.32 | 8.75 | 9.2 | 10.26 | 8.4 | 8.91 | 17 |
| 4 | 9.99 | 9.86 | 11.5 | 13.94 | 7.83 | 15.2 | 13.76 |
| 6 | 14.57 | 13.12 | 15.68 | 14.4 | 13.2 | 16.38 | 20.58 |
| 8 | 16.74 | 31.39 | 22.4 | 24.2 | 18.24 | 25.8 | 16.2 |
| 11 | 13.32 | 39 | 20.65 | 32.4 | 26.1 | 35.51 | 25.42 |
| 13 | 25.52 | 43.86 | 28.88 | 51 | 31.36 | 48.75 | 25.2 |
| 15 | 23.65 | 79 | 38.7 | 72.16 | 35.4 | 68.08 | 30.24 |
| 18 | 37.26 | 133.75 | 86.62 | 104.03 | 71.44 | 108.78 | 22.79 |
| 20 | 44.28 | 140.4 | 102.4 | 147.2 | 102.83 | 153.72 | 24.91 |
| 22 | | | 141 | | 138.6 | | |
| 25 | 77.52 | | 173.03 | | 182.9 | | 11.55 |
| 28 | 95.04 | | | | | | 7.26 |
| 32 | 155.4 | | | | | | 0 |
| 34 | | | | | | | 0 |
| 39 | | | | | | | 0 |
| 41 | | | | | | | 0 |
| 43 | | | | | | | |

Anti-PD-1 (200 ug)+Vehicle

| Day | Mouse 1 | Mouse 2 | Mouse 3 | Mouse 4 | Mouse 5 | Mouse 6 | Mouse |
|---|---|---|---|---|---|---|---|
| 0 | 4.86 | 11.22 | 10.56 | 14.26 | 15.75 | 15.12 | |
| 4 | 15.36 | 24.7 | 19.76 | 23.46 | 21.84 | 30.25 | |
| 6 | 18.4 | 29.11 | 20.91 | 32.34 | 20.52 | 37.62 | |
| 8 | 27.93 | 54.12 | 26.52 | 44.02 | 26.4 | 44.2 | |
| 11 | 32.5 | 75.19 | 28.91 | 35.96 | 36.19 | 59.25 | |
| 13 | 32.9 | 101.76 | 33.64 | 40.96 | 44.88 | 74.62 | |
| 15 | 51.35 | 124.63 | 43.56 | 44.1 | 58.56 | 96.8 | |
| 18 | 61.1 | 186.69 | 32.33 | 43.2 | 58.56 | 148.83 | |
| 20 | 62.4 | | 31.49 | 46.8 | 68.16 | 192.72 | |
| 22 | 64 | | 27.47 | 30.16 | 66.56 | | |
| 25 | 93.15 | | 17.2 | 41.6 | 87.01 | | |
| 28 | 112.14 | | 12.6 | 55.8 | 107.52 | | |
| 32 | 130.81 | | 11.55 | 53.46 | 172.62 | | |
| 34 | 163.9 | | 9.24 | 65.86 | | | |
| 39 | | | 6.44 | 106.7 | | | |
| 41 | | | 5.76 | 152.28 | | | |
| 43 | | | | | | | |

Anti-PD-1 (200 ug)+SYN-115 (30 mg/kg)

| Day | Mouse 1 | Mouse 2 | Mouse 3 | Mouse 4 | Mouse 5 | Mouse 6 | Mouse |
|---|---|---|---|---|---|---|---|
| 0 | 11.2 | 7.28 | 7.28 | 11.2 | 14.5 | 13.23 | |
| 4 | 9.24 | 8.96 | 8.5 | 13.32 | 13.6 | 24.8 | |
| 6 | 8.75 | 5.72 | 4.18 | 5.72 | 19.08 | 23.4 | |
| 8 | 15.84 | 9.8 | 3.36 | 7.5 | 20.3 | 25.65 | |
| 11 | 15.2 | 8.41 | 0 | 0 | 21.5 | 26.4 | |
| 13 | 14.76 | 11.47 | 0 | 0 | 24.96 | 39.53 | |
| 15 | 18.13 | 14.43 | 0 | 0 | 30.68 | 43.07 | |
| 18 | 15.12 | 6.72 | 0 | 0 | 33.55 | 39.33 | |
| 20 | 5.29 | 5.72 | 0 | 0 | 21.12 | 34.98 | |
| 22 | 10.08 | 0 | 0 | 0 | 23.5 | 28.16 | |
| 25 | 0 | 0 | 0 | 0 | 18.06 | 24.44 | |
| 28 | 0 | 0 | 0 | 0 | 16.4 | 21.84 | |
| 32 | 0 | 0 | 0 | 0 | 16.8 | 10.5 | |
| 34 | 0 | 0 | 0 | 0 | 18.8 | 9.92 | |
| 39 | 0 | 0 | 0 | 0 | 33.06 | 0 | |
| 41 | 0 | 0 | 0 | 0 | 29.68 | 0 | |
| 43 | | | | | | | |

Anti-PD-1 (200 ug)+SYN-115 (100 mg/kg)

| Day | Mouse 1 | Mouse 2 | Mouse 3 | Mouse 4 | Mouse 5 | Mouse 6 | Mouse |
|---|---|---|---|---|---|---|---|
| 0 | 8.68 | 10.56 | 9.92 | 14.82 | 17.2 | 12.8 | |
| 4 | 5.72 | 8.19 | 14.57 | 18.9 | 8.84 | 17.68 | |
| 6 | 6.5 | 12.42 | 13.65 | 20.09 | 0 | 19.08 | |
| 8 | 7.59 | 10.5 | 13.68 | 22.4 | 0 | 23.79 | |
| 11 | 9.52 | 3.99 | 7.44 | 21.93 | 0 | 19.8 | |
| 13 | 10.56 | 4.83 | 8.64 | 29.5 | 0 | 21.93 | |
| 15 | 9.24 | 15.81 | 8.4 | 31.86 | 0 | 19.2 | |
| 18 | 5.76 | 28.8 | 4.75 | 39.27 | 0 | 7.98 | |
| 20 | 0 | 32.9 | 0 | 30.25 | 0 | 9.1 | |
| 22 | 0 | 47.04 | 0 | 40.56 | 0 | 0 | |
| 25 | 0 | 85.6 | 0 | 40.3 | 0 | 0 | |
| 28 | 0 | 169.16 | 0 | 50.16 | 0 | 0 | |
| 32 | 0 | | 0 | 76 | 0 | 0 | |
| 34 | 0 | | 0 | 70.62 | 0 | 0 | |
| 39 | 0 | | 0 | 115.92 | 0 | 0 | |
| 41 | 0 | | 0 | 135.36 | 0 | 0 | |
| 43 | | | | 153 | | | |

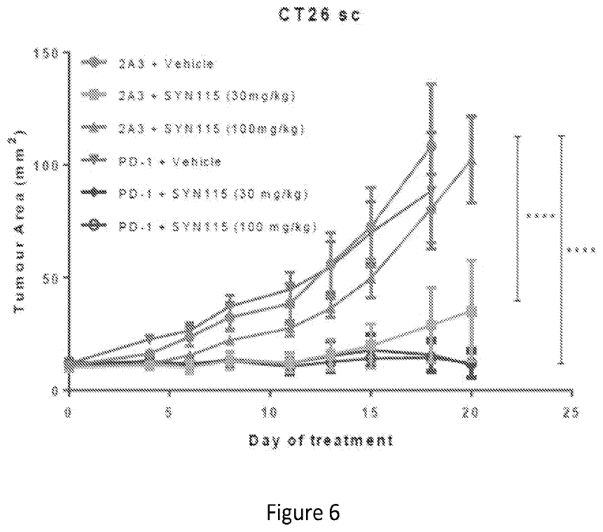

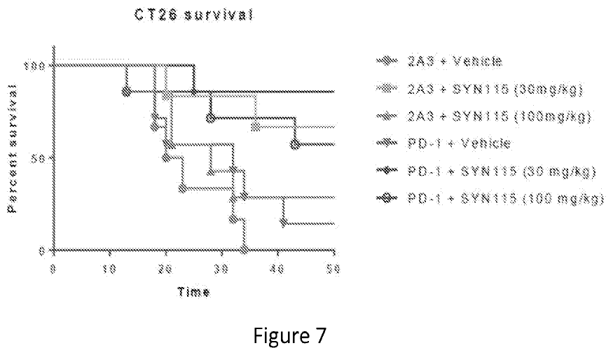

Results are shown as the mean±SEM of n=5-7 mice per group in FIGS. 6 and 7. Statistical test was a Two-Way ANOVA/Bonferroni's multiple comparison test. * * * *p<0.001

### Conclusions

Treatment with SYN-115 at 30 mg/kg resulted in a reduction in tumor growth both as a single agent and in combination with anti-PD-1, leading to long-term survival of a proportion of mice.

### REFERENCES

1. Willingham S B, Ho P Y, Hotson A, Hill C M, Piccione E C, Hsieh J, Liu L, Buggy J J, McCaffery I, Miller R A. A2AR Antagonism with CPI-444 Induces Anti-Tumor Responses and Augments Efficacy to Anti-PD-(L)1 and Anti-CTLA-4 in Preclinical Models. Cancer Immunol. Res. 2018

### 7.6 Example 6: Evaluation of Anti-Tumor Immune Responses of SYN-115 in the CT26 Tumor Mouse Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×10⁵ cells were resuspended in PBS and injected s.c. into BALB/c mice in a 100 μL volume. At day 7 tumors were measured and mice were randomized into distinct treatment groups to ensure that each group had tumors of similar size at the onset of treatment. Mice were treated i.p. at days 7, 11 and 15, with either isotype control (2A3; 200 μg/mouse) or anti-PD-1 mAb (RMP1-14, 200 μg/mouse) and where indicated SYN-115 (Batch 71236AA006) at the indicated dose or vehicle control once-daily via oral gavage on days 7 to 20. To calculate a dose of SYN-115 mouse weight was estimated to be 20 g per mouse. Therefore, 100 mg/kg=2 mg per mouse in 200 which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

Results:

Raw Tumor Measurements:

2A3 (200 μg/Mouse)/Vehicle

| Day | Mouse 1 | Mouse 2 | Mouse 3 | Mouse 4 | Mouse 5 |
|---|---|---|---|---|---|
| 0 | 7.83 | 4.08 | 13.44 | 8.4 | 1 |
| 4 | 16.34 | 5.25 | 19.74 | 24.19 | 18 |
| 7 | 22.26 | 17.76 | 40.96 | 35.91 | 44.8 |
| 11 | 29.28 | 32.86 | 64.86 | 86.86 | 91.67 |
| 14 | 36.18 | 49 | 96.9 | 133.4 | 143 |
| 18 | 55.3 | 79.21 | 120 | | |

2A3 (200 μg/Mouse)+SYN-115 (30 mg/kg)

| Day | Mouse 1 | Mouse 2 | Mouse 3 | Mouse 4 | Mouse 5 |
|---|---|---|---|---|---|
| 0 | 4.08 | 4.25 | 2.52 | 7.54 | 11.22 |
| 4 | 4.56 | 5.4 | 0 | 18.5 | 18.06 |
| 7 | 13.44 | 25.42 | 0 | 29 | 22.26 |
| 11 | 17.34 | 50.96 | 0 | 56.95 | 26.88 |
| 14 | 33.5 | 71.28 | 0 | 81.6 | 36.96 |
| 18 | 50.4 | 111.6 | 0 | 140.98 | 55.3 |

2A3 (200 μg/Mouse)+SYN-115 (100 mg/kg)

| Day | Mouse 1 | Mouse 2 | Mouse 3 | Mouse 4 | Mouse 5 |
|---|---|---|---|---|---|
| 0 | 5.29 | 10.88 | 7.02 | 8.75 | 3.42 |
| 4 | 15.51 | 12.71 | 16 | 9.61 | 6.44 |
| 7 | 24.44 | 17.55 | 19.14 | 23.4 | 9.02 |
| 11 | 43.52 | 35.96 | 52.8 | 43.47 | 8.58 |
| 14 | 68.62 | 75.05 | 92.4 | 79.05 | 14.84 |
| 18 | 116.4 | 102.12 | 162.26 | 134.47 | 32.64 |

Anti-PD-1 (200 ug)+Vehicle

| Day | Mouse 1 | Mouse 2 | Mouse 3 | Mouse 4 | Mouse 5 |
|---|---|---|---|---|---|
| 0 | 8.99 | 3.45 | 6.9 | 3.99 | 5.29 |
| 4 | 16.47 | 10.44 | 23.92 | 11.88 | 15.12 |
| 7 | 29.4 | 20.8 | 39.42 | 22.55 | 30.09 |
| 11 | 41.65 | 18.06 | 69 | 32.2 | 40.12 |
| 14 | 43.24 | 27.26 | 89.28 | 45.36 | 50.32 |
| 18 | 46.64 | 44.85 | 182.21 | 87.78 | 55.76 |

Anti-PD-1 (200 ug)+SYN-115 (30 mg/kg)

| Day | Mouse 1 | Mouse 2 | Mouse 3 | Mouse 4 | Mouse 5 |
|---|---|---|---|---|---|
| 0 | 9.18 | 3.6 | 6.5 | 10.24 | 5.44 |
| 4 | 27.03 | 9.99 | 11.48 | 14.4 | 15.37 |
| 7 | 70.5 | 18 | 17.6 | 29.58 | 41.18 |
| 11 | 90.48 | 20 | 29.12 | 56 | 61.62 |
| 14 | 131.13 | 20.24 | 20.24 | 71.89 | 86.86 |
| 18 | | 19.6 | 19.27 | 88 | 128.82 |

Anti-PD-1 (200 ug)+SYN-115 (100 mg/kg)

| Day | Mouse 1 | Mouse 2 | Mouse 3 | Mouse 4 | Mouse 5 |
|---|---|---|---|---|---|
| 0 | 9.8 | 3.8 | 5.5 | 9.92 | 5.8 |
| 4 | 12.48 | 27.95 | 9.9 | 15.21 | 12.8 |
| 7 | 17.48 | 51.94 | 32.64 | 14.44 | 14.85 |
| 11 | 17.16 | 78.84 | 62.4 | 8.96 | 38.28 |
| 14 | 26.22 | 108.24 | 104.5 | 10.56 | 60.48 |
| 18 | 39.44 | | | 4.64 | 113.28 |

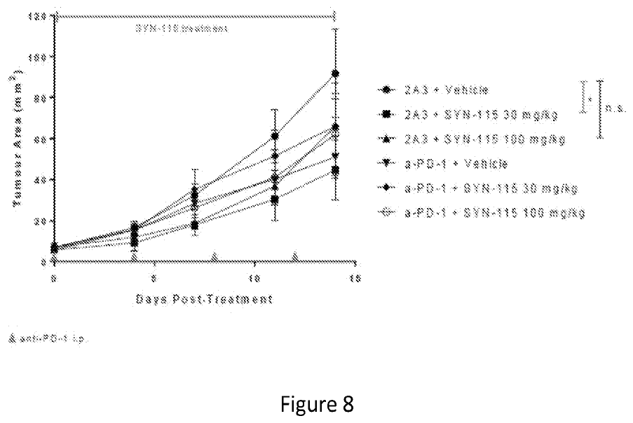

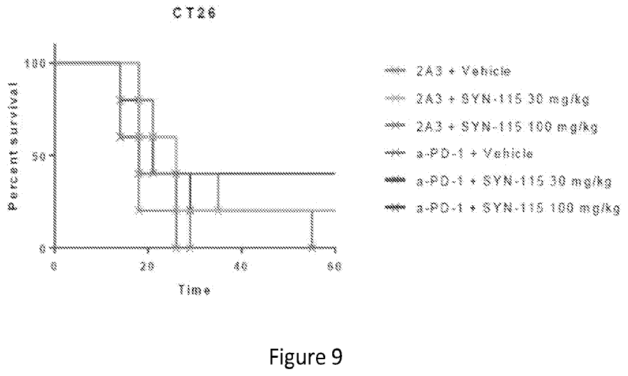

Results are shown as the mean±SEM of n=5 mice per group in FIGS. 8 and 9. Statistical test was a Two-Way ANOVA/Bonferroni's multiple comparison test. *p<0.05

### Conclusions

Single agent activity was observed with SYN-115 at 30 mg/kg but not 100 mg/kg. However, no increased anti-tumor response was observed in combination with anti-PD-1.

### REFERENCES

1. Willingham S B, Ho P Y, Hotson A, Hill C M, Piccione E C, Hsieh J, Liu L, Buggy J J, McCaffery I, Miller R A. A2AR Antagonism with CPI-444 Induces Anti-Tumor Responses and Augments Efficacy to Anti-PD-(L)1 and Anti-CTLA-4 in Preclinical Models. Cancer Immunol. Res. 2018

### 7.7 Example 7: Evaluation of Anti-Tumor Immune Responses of SYN-115 in the CT26 Tumor Mouse Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×10⁵ cells were resuspended in PBS and injected s.c. into BALB/c mice in a 100 μL volume. At day 7 tumors were measured and mice were randomized into distinct treatment groups to ensure that each group had tumors of similar size at the onset of treatment. Mice were treated with SYN-115 (Batch 71236AA006) at the indicated dose or vehicle control once-daily via oral gavage on days 7 to 20. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C. Tumor measurements were taken 2-3 times per week. Tumor sizes were determined by width * length and mice were culled when tumors reached the ethical limit of 150 mm².

Results:

Raw Tumor Measurements:

Vehicle

| Day | Mouse 1 | Mouse 2 | Mouse 3 | Mouse 4 | Mouse 5 |
|---|---|---|---|---|---|
| 0 | 3.61 | 8.5 | 6.75 | 8.5 | 10.15 |
| 2 | 9.24 | 11.22 | 13.12 | 11.84 | 15.84 |
| 4 | 42.88 | 25.2 | 19.76 | 20.91 | 19.6 |
| 7 | 58.48 | 48.36 | 29 | 38.19 | 28.8 |
| 9 | 120.28 | 71.1 | 35.51 | 54 | 44.25 |
| 11 | 168.72 | 85.68 | 55.68 | 81 | 54 |
| 14 | 225.76 | 131.84 | 85.02 | 126.42 | 79.18 |
| 16 | | 154.88 | 111.93 | 155 | 105.84 |
| 18 | | | 158.46 | | 126.04 |
| 21 | | | | | 154.96 |

SYN-115 (1 mg/kg)

| Day | Mouse 1 | Mouse 2 | Mouse 3 | Mouse 4 | Mouse 5 | Mouse 6 |
|---|---|---|---|---|---|---|
| 0 | 5.25 | 6.72 | 8.37 | 7.84 | 9.6 | 10.36 |
| 2 | 12.95 | 12.54 | 15.6 | 13.6 | 12.71 | 15.84 |
| 4 | 14.8 | 22.95 | 21 | 24.99 | 20.4 | 24.19 |
| 7 | 53.2 | 40.6 | 33.04 | 39.9 | 25.65 | 35.64 |
| 9 | 97.96 | 56.58 | 45.56 | 76.44 | 36.85 | 60.2 |
| 11 | 144.9 | 74.1 | 48.3 | 103.5 | 40.12 | 86.7 |
| 14 | 238.52 | 116 | 84 | 172.5 | 87 | 113.22 |
| 16 | | 144.1 | 103.55 | | 103.74 | 155.61 |
| 18 | | 208.68 | 167.24 | | 138.6 | |
| 21 | | | | | 173.46 | |

SYN-115 (3 mg/kg)

| Day | Mouse 1 | Mouse 2 | Mouse 3 | Mouse 4 | Mouse 5 |
|---|---|---|---|---|---|
| 0 | 4.32 | 6.67 | 8.4 | 9.24 | 11 |
| 2 | 8.99 | 13.32 | 17.48 | 10.15 | 8.7 |
| 4 | 8.06 | 23.32 | 24.08 | 17.22 | 6.09 |
| 7 | 9.1 | 31.05 | 34.45 | 21.42 | 4.18 |
| 9 | 13.32 | 27.52 | 45.5 | 25.97 | 3.8 |
| 11 | 19.8 | 28.98 | 66.42 | 29.58 | 7 |
| 14 | 43.56 | 44.55 | 111.1 | 51 | 2.7 |
| 16 | 50.32 | 50.73 | 112.86 | 72.98 | 3.04 |
| 18 | 74.62 | 69.01 | 134.4 | 96.12 | 0 |
| 21 | 118.56 | 97.11 | 179.8 | 157.76 | 0 |
| 25 | 175.44 | 171.36 | | | 0 |

SYN-115 (5 mg/kg)

| Day | Mouse 1 | Mouse 2 | Mouse 3 | Mouse 4 |
|---|---|---|---|---|
| 0 | 4.8 | 7.8 | 7.54 | 8.84 |
| 2 | 13.12 | 11.16 | 13.26 | 10.36 |
| 4 | 20.8 | 11.2 | 18.45 | 19.52 |
| 7 | 28.98 | 13.3 | 13.3 | 36 |
| 9 | 32.9 | 21.93 | 25.44 | 54.6 |
| 11 | 45.24 | 27.56 | 34.8 | 68 |
| 14 | 75.84 | 52.36 | 43.8 | 112.32 |
| 16 | 95.68 | 63.18 | 42.84 | 147.06 |
| 18 | 97.65 | 111.28 | 64.74 | 208.6 |
| 21 | 135.42 | 156 | 73.1 | |
| 25 | 163.8 | | 83.52 | |
| 29 | | | 101.76 | |
| 32 | | | 124.74 | |
| 35 | | | 146.52 | |
| 37 | | | 161.59 | |

SYN-115 (10 mg/kg)

| Day | Mouse 1 | Mouse 2 | Mouse 3 | Mouse 4 | Mouse 5 | Mouse 6 |
|---|---|---|---|---|---|---|
| 0 | 4.86 | 6.44 | 7.56 | 8.37 | 8.84 | 11.2 |
| 2 | 6.16 | 6.75 | 7.36 | 15.2 | 9.45 | 14.06 |
| 4 | 15.75 | 12.16 | 22.55 | 23.52 | 8.36 | 26.95 |
| 7 | 20.7 | 14.04 | 41.44 | 34.77 | 6.24 | 46.92 |
| 9 | 30.53 | 22.09 | 52.08 | 50.37 | 0 | 66.99 |
| 11 | 44.1 | 31.32 | 70.68 | 66.42 | 0 | 89.24 |
| 14 | 52.8 | 48.96 | 108.1 | 114.45 | 0 | 125.08 |
| 16 | 91.44 | 67.86 | 132.84 | 135.42 | 0 | 179.55 |
| 18 | 136.71 | 100.98 | 202.5 | 119.88 | 0 | |
| 21 | 175.74 | 162 | | 160 | 0 | |
| 25 | | | | | 0 | |
| 29 | | | | | 0 | |
| 32 | | | | | 0 | |
| 35 | | | | | 0 | |
| 37 | | | | | 0 | |

SYN-115 (30 mg/kg)

| Day | Mouse 1 | Mouse 2 | Mouse 3 | Mouse 4 | Mouse 5 | Mouse 6 |
|---|---|---|---|---|---|---|
| 0 | 5 | 6.24 | 7.56 | 8.7 | 12.48 | 5 |
| 2 | 12.6 | 8.12 | 9.28 | 12.95 | 8.4 | 12.6 |
| 4 | 21.6 | 15.96 | 13.65 | 24.48 | 10.56 | 21.6 |
| 7 | 42.21 | 25 | 25.5 | 30.21 | 30.24 | 42.21 |
| 9 | 50.16 | 35.4 | 29.68 | 47.61 | 31.9 | 50.16 |
| 11 | 68.53 | 49 | 54.72 | 73.95 | 50.37 | 68.53 |
| 14 | 110.11 | 63.64 | 87.36 | 99.75 | 44.84 | 110.11 |
| 16 | 140.8 | 95.68 | 114.13 | 132.5 | 77.08 | 140.8 |
| 18 | 204.8 | 114 | 189.23 | 202.86 | 125.4 | 204.8 |
| 21 | | 153.4 | | | 179.4 | |

SYN-115 (50 mg/kg)

| Day | Mouse 1 | Mouse 2 | Mouse 3 | Mouse 4 | Mouse 5 |
|---|---|---|---|---|---|
| 0 | 5.2 | 6.09 | 6.6 | 8.68 | 13.53 |
| 2 | 14.44 | 12.48 | 11.7 | 13.02 | 8.68 |
| 4 | 17.2 | 21.84 | 14.4 | 29.4 | 24.01 |
| 7 | 30.78 | 39.5 | 19.95 | 60.9 | 38.43 |
| 9 | 44.73 | 65.8 | 22.04 | 97.02 | 43.52 |
| 11 | 60.06 | 100.1 | 22.2 | 117.66 | 59.94 |
| 14 | 94.5 | 145.77 | 28.35 | 201.55 | 85.54 |
| 16 | 125.13 | 165.2 | 34.45 | | 94.76 |
| 18 | 161.66 | | 60.06 | | 133.2 |
| 21 | | | 68.68 | | 186.12 |
| 25 | | | 169.36 | | |

SYN-115 (100 mg/kg)

| Day | Mouse 1 | Mouse 2 | Mouse 3 | Mouse 4 | Mouse 5 | Mouse 6 |
|---|---|---|---|---|---|---|
| 0 | 5.22 | 5.72 | 6.72 | 7.29 | 8.64 | 13.68 |
| 2 | 8.12 | 9.24 | 10.36 | 9.28 | 18.8 | 12.8 |
| 4 | 13.12 | 16.5 | 22.96 | 19.2 | 24.48 | 19.6 |
| 7 | 21.6 | 46.86 | 27.73 | 29.58 | 48.28 | 43.32 |
| 9 | 35.19 | 79.17 | 43.66 | 41.3 | 69.66 | 80.58 |
| 11 | 41.25 | 116.55 | 53.12 | 51 | 100 | 109.61 |
| 14 | 60.52 | 200.2 | 68.87 | 70.84 | 160.48 | 176.85 |
| 16 | 80.36 | | 103.53 | 86.52 | | |
| 18 | 106.56 | | 118.8 | 106 | | |
| 21 | 158.73 | | 166.75 | 125.35 | | |
| 25 | | | | 177.32 | | |

SYN-115 (100 mg/kg 4 days on, 3 days off: drug holiday)

| Day | Mouse 1 | Mouse 2 | Mouse 3 | Mouse 4 | Mouse 5 | Mouse |
|---|---|---|---|---|---|---|
| 0 | 3.78 | 7.83 | 5.4 | 7.54 | 7.8 | 16.28 |
| 2 | 2.88 | 12.3 | 7.56 | 9.92 | 6.67 | 19.11 |
| 4 | 4.8 | 18.9 | 15.98 | 12.18 | 20.4 | 29.07 |
| 7 | 16.81 | 24.94 | 35.28 | 34.1 | 34.45 | 42.78 |
| 9 | 25.85 | 33.63 | 52.92 | 47.12 | 57.27 | 60.68 |
| 11 | 35.75 | 40.87 | 71.28 | 67.76 | 82.65 | 79.68 |
| 14 | 69.16 | 55.3 | 121.68 | 105.3 | 100.57 | 114.46 |
| 16 | 82.56 | 69.7 | 164.7 | 125.24 | 118.8 | 133.28 |
| 18 | 117.81 | 100.88 | | 172.55 | 132.6 | 193.88 |
| 21 | 200.66 | 110.21 | | | 176.9 | |
| 25 | | 153.67 | | | | |

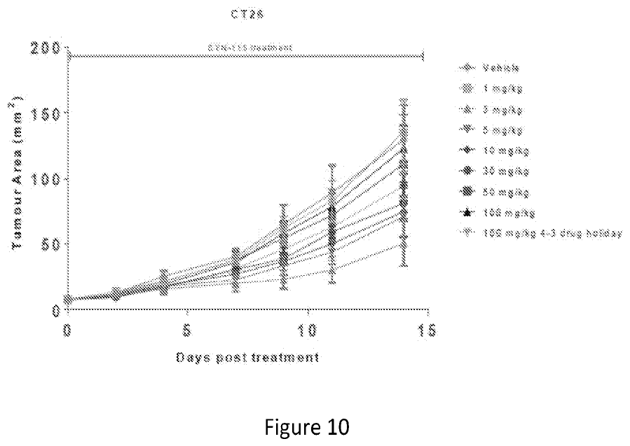

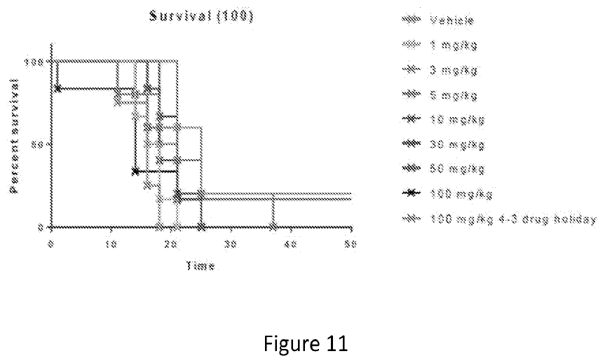

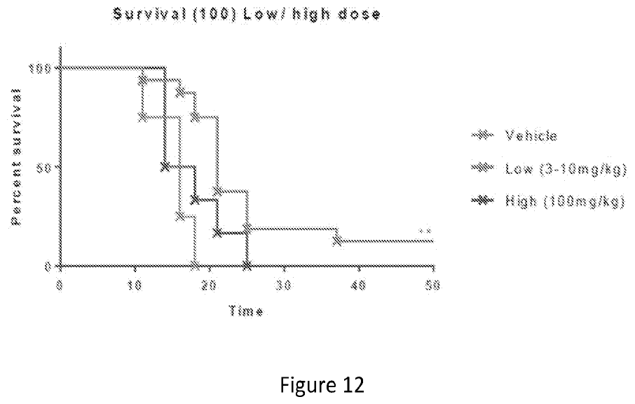

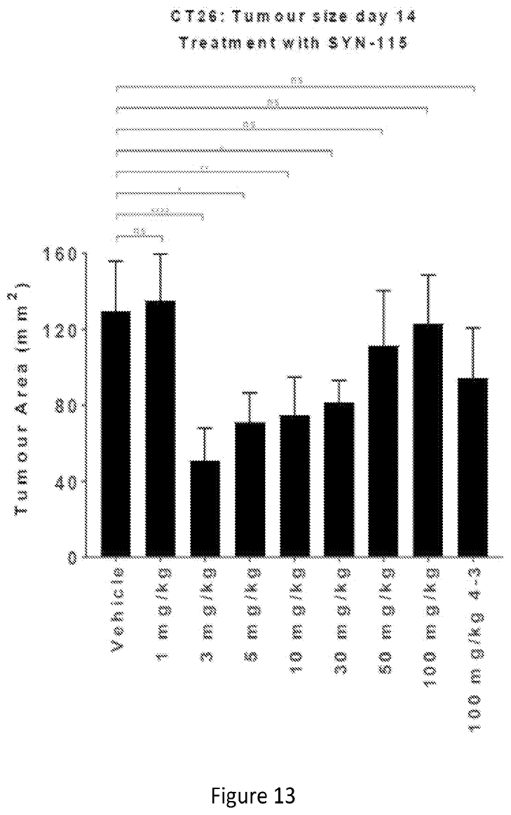

Results are shown as the mean±SEM of n=4-6 mice per group in FIGS. 10-13.

Statistical test was a Two-Way ANOVA/Bonferroni's multiple comparison test. *p<0.05 **p<0.01 ****p<0.0001. Comparison of survival curves was a Log-rank (Mantel-Cox) test **p<0.01

### Conclusions

SYN-115 treatment resulted in significantly reduced tumor growth, particularly at lower doses of SYN-115. There was a clear distinction in the survival of mice treated with low dose (3 mg/kg-10 mg/kg) vs high dose (100 mg/kg) SYN-115.

### 7.8 Example 8: Evaluation of Anti-Tumor Metastasis of SYN-115 in the B16F10 CD73 Tumor Mouse Model

Methods:

The C57/BL6 mouse melanoma cell line B16F10 CD73 was cultured in DMEM supplemented with 10% FCS, GlutaMAX, and penicillin/streptomycin. Tumor cells are periodically verified to be Mycoplasma negative by the Victorian Infectious Diseases References

Lab (Melbourne, VIC, Australia) by PCR analysis. 2×10⁵ cells were resuspended in PBS and injected i.v. into C57/BL6 mice (A_{2A}^{-/-} or Wild-Type) in a 200 μL volume. At day 14, lungs were excised and the number of metastases counted under the dissecting microscope. Mice were dosed with either SYN-115 (Batch 71236AA002) at 100 mg/kg or vehicle control once-daily via oral gavage on days 0 to 13. To calculate a dose of SYN-115, mouse weight was estimated to be 20 g per mouse. Therefore, 100 mg/kg=2 mg per mouse in 200 μL, which is equivalent to 10 mg/ml. To make up 40 mL 400 milligrams of SYN-115 was weighed and added into a Falcon tube, then 1 mL of Tween 80 added and mixed into a paste and then 39 mL of 1% Methyl Cellulose was added. Solution was vortexed and then left on rotating mixer for at least 2 hours and maintained at 4° C.

Results:

Number of Tumor Metastases:

| | | SYN- | A2A^{-/-} |
|---|---|---|---|
| | Vehicle | 115 | Vehicle |
| Mouse 1 | 54 | 5 | 10 |
| Mouse 2 | 34 | 11 | 13 |
| Mouse 3 | 48 | 1 | 13 |
| Mouse 4 | 57 | 13 | |
| Mouse 5 | 43 | 28 | |
| Mouse 6 | 59 | 2 | |
| Mouse 7 | 22 | 8 | |
| Mouse 8 | 27 | 21 | |
| Mouse 9 | 74 | 1 | |
| Mouse 10 | 33 | | |

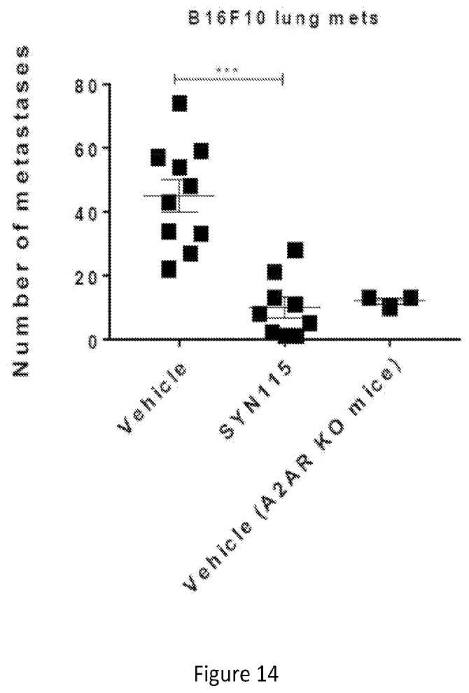

Results are shown as the mean±SEM of n=3-10 mice per group in FIG. 14. Statistical significance was performed using an unpaired t Test.

### Conclusions

SYN-115 elicited potent anti-metastatic activity with metastatic burden similar to that observed in A_{2A}^{-/-} mice. This was a more striking reduction in tumor metastasis compared to historical data using the A_{2A} receptor antagonist SCH58261 when administered at a dose of 1 mg/kg (Beavis et al. 2013).

### REFERENCES

1. Beavis P A, Divisekera U, Paget C, Chow M T, John L B, Devaud C, Dwyer K, Stagg J, Smyth M J, Darcy P K. (2013), Blockade of A2A receptors potently suppresses the metastasis of CD73+ tumors, PNAS 110(36):14711-6

### 7.9 Example 9: Evaluation of SYN-15 Effects on Anti-Tumor Metastasis in the B16F10 CD73 Tumor Mouse Model

### Methods:

The C57/BL6 mouse melanoma cell line B16F10 CD73 was cultured in DMEM supplemented with 10% FCS, GlutaMAX, and penicillin/streptomycin. Tumor cells are periodically verified to be Mycoplasma negative by the Victorian Infectious Diseases References

Lab (Melbourne, VIC, Australia) by PCR analysis. 2×10⁵ cells were resuspended in PBS and injected i.v. into C57/BL6 mice (A_{2A}^{-/-} or Wild-Type) in a 200 μL volume. At day 14 lungs were excised and the number of metastases counted under the dissecting microscope. Mice were dosed with either SYN-115 (Batch 71236AA002) at 100 mg/kg or vehicle control once-daily via oral gavage on days 0 to 13. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C.

### Results:

Number of Tumor Metastases:

| | | SYN- | A2A^{-/-} |
|---|---|---|---|
| | Vehicle | 115 | Vehicle |
| Mouse 1 | 38 | 61 | 0 |
| Mouse 2 | 74 | 72 | 0 |
| Mouse 3 | 64 | 64 | 3 |
| Mouse 4 | 89 | 40 | 0 |
| Mouse 5 | 78 | 24 | |
| Mouse 6 | 13 | 56 | |
| Mouse 7 | 46 | 82 | |
| Mouse 8 | 84 | 53 | |
| Mouse 9 | 61 | 108 | |
| Mouse | 132 | 107 | |
| 10 | | | |

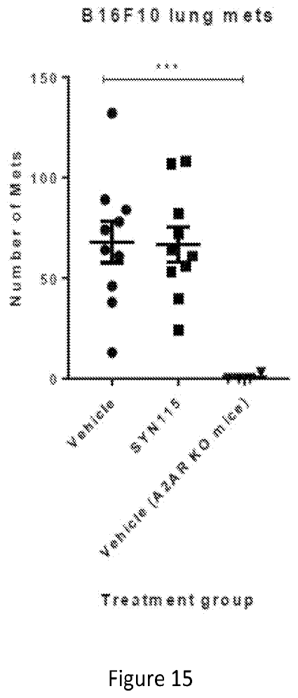

Results are shown as the mean±SEM of n=4-10 mice per group in FIG. 15. Statistical significance was performed using an unpaired t Test.

### Conclusions

No efficacy of the SYN-115 was observed in this experiment, although A2AKO mice were significantly protected as expected. A new batch of SYN-115 was therefore requested for future experiments. The new batch was used in Examples 10, 11, 12 and 13.

### 7.10 Example 10: Evaluation of Anti-Tumor Metastasis of SYN-115 in the B16F10 CD73 Tumor Mouse Model

### Methods:

The C57/BL6 mouse melanoma cell line B16F10 Cherry was cultured in DMEM supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 2×10⁵ cells were resuspended in PBS and injected i.v. into C57/BL6 mice in a 200 μL volume. At day 14 lungs were excised and the number of metastases counted under the dissecting microscope. Mice were dosed with either SYN-115 (Batch 71236AA006) at 100 mg/kg or vehicle control once-daily via oral gavage on days 0 to 13. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. Solution was vortexed and then left on a rotating mixer for at least 2 hours and maintained at 4° C.

Results:

Number of tumor metastases:

| AC#009 Males | | | AC#010 Females | | |
|---|---|---|---|---|---|
| | Vehicl [symbol] | SYN- [symbol] | | Vehicl [symbol] | SYN- [symbol] |
| Mouse 1 | 28 | 0 | Mouse 1 | 381 | 141 |
| Mouse 2 | 60 | 9 | Mouse 2 | 277 | 32 |
| Mouse 3 | 65 | 5 | Mouse 3 | 76 | 137 |
| Mouse 4 | 12 | 16 | Mouse 4 | 202 | 264 |
| Mouse 5 | 80 | 13 | Mouse 5 | 262 | 23 |
| Mouse 6 | 39 | 1 | Mouse 6 | 108 | 226 |
| Mouse 7 | 8 | 0 | Mouse 7 | 258 | 166 |
| Mouse 8 | 48 | | Mouse 8 | | 38 |

[symbol]

indicates data missing or illegible when filed

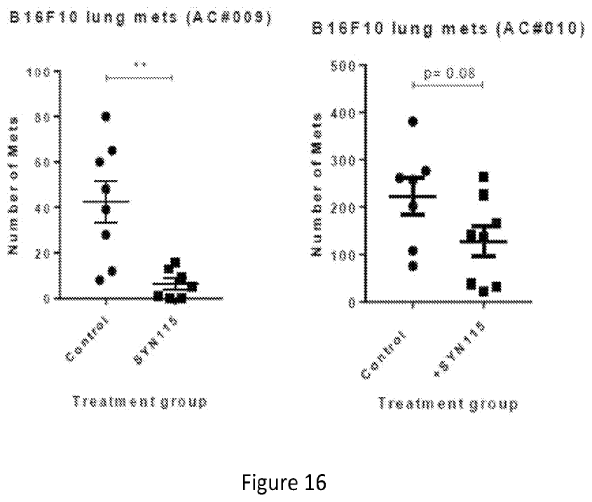

Results are shown as the mean±SEM of n=7-8 mice per group in FIG. 16. Statistical significance was performed using an unpaired t Test. **p<0.01

### Conclusions

SYN-115 reduced tumor metastases in both experiments. These experiments indicated that the batch of SYN-115 being used was active.

### 7.11 Example 11: Evaluation of Pharmacokinetics of Tozadenant in Mice

The plasma pharmacokinetics and exposure levels of tozadendant have been documented in mice after oral administration, the same route of administration as tozadenant was dosed in the oncology efficacy studies.

The PK of tozadenant in mice was compared across a range of doses and dose frequencies. SYN115 was administered as two doses, given either 0 and 8 hours (males only) or 0 and 12 hours (males and females at the high dose) apart.

Groups of mice of the Crl:CD1(ICR) strain were dosed, as follows:

Mouse PK Study Design

| | | | | | Number of animals in | |
|---|---|---|---|---|---|---|
| | | Dose level | Dose level | Commencement | group | |
| Group | Group | (mg/kg/ | (mg/kg/ | of dosing | Pharmacokinetics | |
| Number | Description | dose)¹ | day) | day² | Male | Female |
| 1 | Low 1 | 50 | 100 | 1 | 16 | — |
| 2 | Intermediate 1 | 150 | 300 | 3 | 16 | — |
| 3 | Intermediate 2 | 500 | 1000 | 5 | 16 | — |
| 4 | High 1-BID 8 | 1000 | 2000 | 8 | 16 | 16 |
| | hour | | | | | |
| 5 | High 2-BID 12 | 1000 | 2000 | 10 | 16 | 16 |
| | hour | | | | | |

¹Groups 1-3 (males) and 4 (males and females) were dosed BID at 0, 8 hours for 1 day. Group 5 (males and females) were dosed BID at 0, 12 hours. PK sampling times were adjusted for the 8 and 12 hour dosing groups in order to provide two symmetrical pharmacokinetic profiles over 24 hours, enabling comparison of Cₘₐₓ and Cₘᵢₙ, after the first and second doses. No vehicle control group was included, as this was a PK study.
²Dosing for each group commenced two or three days after the last group was dosed

The vehicle for the tozadenant in this mouse PK study was 1.6% (w/v) Avicel RC-591 (FMC), 0.2% (v/v) Tween 80, 0.18% (w/v) methylparaben and 0.02% (w/v) propylparaben in reverse osmosis water, pH 6.0±0.3. The 1.6% (w/v) Avicel RC-591 (FMC) was supplied by Biopolymer International, Brussels, Belgium. The 0.2% (v/v) Tween 80, 0.18% (w/v) methylparaben and 0.02% (w/v) propylparaben in reverse osmosis water, pH 6.0±0.3 was supplied by and Sigma-Aldrich Co Ltd, Poole, UK. This is the same formulation used for the mouse oncology model oral dosing.

The dose formulations were stored refrigerated at 2 to 8° C. in a sealed container. On arrival at the animal room, the dose formulations were stirred using a magnetic stir bar and stir plate for at least 30 minutes prior to dosing. Dose formulations were allowed to reach room temperature prior to dosing, and were stirred continuously throughout dosing.

A sufficient number of mice of the Crl:CD1(ICR) strain were obtained from Charles River Laboratories, Margate, UK. The animals were obtained as weanlings of about 25 to 27 days of age on arrival. The animals were ordered to be within ±20% of the overall mean for each sex on despatch Males weighed between 27.4 and 40.5 g, and females weighed between 23.1 and 31.1 g; the animals were approximately 5 to 7 weeks old at the start of dosing.

The animals were housed in a single, exclusive room, with 15 to 20 air changes/hour. The temperature and relative humidity ranges were set to maintain the specified ranges of 20 to 24° C. and 45 to 65%, respectively. The animals were kept in this environment except for short periods of time where experimental procedures dictated otherwise. Fluorescent lighting was controlled automatically to give a cycle of 12 hours light and 12 hours dark. The light/dark cycle was interrupted for toxicokinetic sample collection procedures. Males were singly housed and females were housed in groups of up to three in cages that conformed with animal care guidelines. Throughout the study the animals had access ad libitum to SQC Rat and Mouse Maintenance Diet No 1, Expanded, (Special Diets Services Ltd, Witham, UK). Each batch of diet was analyzed for specific constituents and contaminants. Mains supply tap water was provided ad libitum via water bottles. The water was periodically analyzed for specific contaminants. Bedding was provided on a weekly basis to each cage by use of clean Aspen wood chips (Datesand Ltd, Manchester, UK). Each batch of bedding was analyzed for specific constituents and contaminants.

The test article was administered orally by gavage, as follows:

Groups 1 to 4: Twice for one day (0 and 8 hours apart relative to initial dose)

Group 5: Twice for one day (0 and 12 hours apart relative to initial dose)

Blood samples for toxicokinetics (0.25 mL nominal) were taken from all toxicokinetic animals (subgroup 2), as follows:

8 hour interval (BID doses at 0 and 8 hours)

| | Animals bled at the | | | | | | |
|---|---|---|---|---|---|---|---|
| Group | following time points (hours post dose): | | | | | | |
| and Sex | 0.5 | 1 | 2 | 4 | 6 | 8¹ | 8.5 |
| 1M | 1-2 | 3-4 | 5-6 | 7-8 | 9-10 | 11-14 | 1-2 |
| 2M | 17-18 | 19-20 | 21-22 | 23-24 | 25-26 | 27-30 | 17-18 |
| 3M | 33-34 | 35-36 | 37-38 | 39-40 | 41-42 | 43-46 | 33-34 |
| 4M | 49-50 | 51-52 | 53-54 | 55-56 | 57-58 | 60-62² | 49-50 |
| 4F | 65-66 | 67-68 | 69-70 | 71-72 | 73-74 | 75-78 | 65-66 |

| | Animals bled at the | | | | | |
|---|---|---|---|---|---|---|
| Group | following time points (hours post dose): | | | | | |
| and Sex | 9 | 10 | 12 | 14 | 16 | 24 |
| 1M | 3-4 | 5-6 | 7-8 | 9-10 | 11-12 | 13-14 |
| 2M | 19-20 | 21-22 | 23-24 | 25-26 | 27-28 | 29-30 |
| 3M | 35-36 | 37-38 | 39-40 | 41-42 | 43-44 | 45-46 |
| 4M | 51-52 | 53-54 | 55-56 | 57-58 | 59-60 | 61-62 |
| 4F | 67-68 | 69-70 | 71-72 | 73-74 | 75-76 | 77-78 |

¹Immediately before second dose.
²Sample not obtained from 59M as the animal was difficult to bleed.

12 hour interval (BID doses at 0 and 12 hours)

| | Animals bled at the | | | | | | |
|---|---|---|---|---|---|---|---|
| Group | following time points (hours post dose): | | | | | | |
| and Sex | 0.5 | 1 | 2 | 4 | 6 | 8 | 12¹ |
| 5M | 81-82 | 83-84 | 85-86 | 87-88 | 89-90 | 91-92 | 93-94 |
| 5F | 97-98 | 99-100 | 101- | 103- | 105- | 107- | 109- |
| | | | 102 | 104 | 106 | 108 | 110 |
| | Animals bled at the | | | | | | |
| Group | following time points (hours post dose): | | | | | | |
| and Sex | 12.5 | 13 | 14 | 16 | 18 | 20 | 24 |
| 5M | 81-82 | 83-84 | 85-86 | 87-88 | 89-90 | 91-92 | 93-94 |
| 5F | 97-98 | 99-100 | 101- | 103- | 105- | 107- | 109- |
| | | | 102 | 104 | 106 | 108 | 110 |

¹Immediately before second dose.

For animals dosed at 0, 8 or 12 hour intervals, sampling time with respect to dose was symmetrical in each animal, for example, if an animal was sampled 0.5 hours after the first dose, it was sampled 0.5 hours after the second dose. The second sample obtained from each animal was collected under terminal anaesthesia.

Samples were taken from the orbital sinus (with the exception of Group 3 animals at the 10 hour bleed, which were taken by cardiac puncture) under isoflurane anaesthesia into K₂EDTA. Samples were gently inverted to ensure mixing with anticoagulant, placed on a blood roller for at least one minute and then placed in a Kryorack until centrifugation.

Centrifugation was carried out at approximately 4-8° C. within one hour of collection. The resultant plasma from all samples was separated, transferred to uniquely labelled clear polypropylene tubes and frozen immediately at approximately <−10 to −30° C. Samples were analyzed at Covance.

Tozadenant was quantitated using protein precipitation followed by liquid chromatography mass spectrometry technique for quantification. This bioanalytical method was previously established and validated for tozadenant in mouse plasma.

The mouse PK parameters obtained from this study are summarized in the table below:

Part A-Single Day: Toxicokinetic parameters of
SYN115 in male and female mice following twice
daily oral administration of SYN115 at 0 and 8 or 12 hours

| Group | 1 | 2 | 3 | 4 | | 5 | |
|---|---|---|---|---|---|---|---|
| Dose | 100 | 300 | 1000 | 2000 | | 2000 | |
| (mg/kg/day) | | | | | | | |
| Dose | 50 | 150 | 500 | 1000 | | 1000 | |
| (mg/kg/dose) | | | | | | | |
| Dosing Times | 0, 8 | 0, 8 | 0, 8 | 0, 8 | | 0, 12 | |
| (hr) | | | | | | | |
| Gender | Male | Male | Male | Male | Female | Male | Female |
| Cₘₐₓ (ng/mL) | 764 | 1720 | 3610 | 5080 | 12100 | 6440 | 8240 |
| Tₘₐₓ (hr) | 9.0 | 8.8 | 10.2 | 9.0 | 14.0 | 1.1 | 1.2 |
| t_{1/2} (hr) | NR | 0.971 | NR | 2.02 | NR | 1.36 | NR |
| AUC₀₋₂₄ | 3520 | 9900 | 29900 | 43000 | 116000 | 31100 | 62700 |
| (ng · h/mL) | | | | | | | |
| AUC₀₋₈ | 1950 | 5030 | 13800 | 20500 | 35400 | — | — |
| (ng · h/mL) | | | | | | | |
| AUC₈₋₂₄ | 1570 | 4870 | 16100 | 22500 | 80600 | — | — |
| (ng · h/mL) | | | | | | | |
| AUC₀₋₁₂ | — | — | — | — | — | 17800 | 35500 |
| (ng · h/mL) | | | | | | | |
| AUC₁₂₋₂₄ | — | — | — | — | — | 13300 | 27200 |
| (ng · h/mL) | | | | | | | |
| AUC_{0-∞} | NR | 9930 | NR | 43100 | NR | 31300 | NR |
| (ng · h/mL) | | | | | | | |
| Cₘₐₓ/D | 7.64 | 5.73 | 3.61 | 2.54 | 6.05 | 3.22 | 4.12 |
| AUC₀₋₂₄/D | 35.2 | 33.0 | 29.9 | 21.5 | 58.0 | 15.6 | 31.4 |

Dose Proportionality (compared to 100 mg/kg/day; 0, 8 hour dosing)

| | | | | | | | |
|---|---|---|---|---|---|---|---|
| Dose increment | — | 3 | 10 | 20 | NA | — | — |
| Increase in Cₘₐₓ | — | 2.25 | 4.73 | 6.65 | NA | — | — |
| Increase in | — | 2.81 | 8.49 | 12.2 | NA | — | — |
| AUC₀₋₂₄ | | | | | | | |

Female:Male Ratio

| | | | | | |
|---|---|---|---|---|---|
| Cₘₐₓ | — | — | — | 2.38 | 1.28 |
| AUC₀₋₂₄ | — | — | — | 2.70 | 2.02 |

n = 2 animals/nominal time point except n = 4 at 8 hours for Groups 1-4
NA—Not Applicable
NR—Not Reported

All animals had measurable plasma concentrations, demonstrating drug absorption at all dose levels. Mean plasma concentrations of tozadenant were at a maximum (Tₘₐₓ) following the second dose with 0, 8 hours dosing, and were between 0.8 and 2.2 hours post dose in all groups with the exception of the females dosed with 2000 mg/kg/day (0, 8 hours dosing), where the Tₘₐₓ was 6 hours after the second dose. Tₘₐₓ occurred following the first dose with 0, 12 hour dosing, and values were 1.1 and 1.2 hours for males and females, respectively. The half-lives of tozadenant (where calculated) did not appear to be dose dependent, and ranged from approximately 1-2 hours. The exposure (Cₘₐₓ and AUC₀₋₂₄) of tozadenant generally increased with each increase in dose level and appeared to be proportional at the lower dose levels and sub-proportional at the higher dose levels. Comparison of the 2 different dose intervals at 2000 mg/kg/day showed higher 24-hour exposures (Cₘₐₓ and AUC₀₋₂₄) following 0, 8 hour dosing than 0, 12 hours, with the exception of Cₘₐₓ in males which was higher following 0, 12 hour dosing. At 2000 mg/kg/day, where the gender effect was evaluated, exposure was consistently higher in females than in males with both 0, 8 and 0, 12 hour dosing. Both the AUC₀₋₂₄ and the Cₘₐₓ female: male ratio were lower in the 0, 12 hour dose interval group than in the 0,8 hour dose interval group, however the difference was only slight for AUC₀₋₂₄.

The dose from this PK study most relevant to the mouse oncology doses is the lowest dose tested, 100 mg/kg/day (50 mg/kg/dose×2) dose. At that dose level, oral dosing of tozadenant at 10 mg/kg in mice resulted in an average Cmax of 764 ng/mL, an average daily AUC₀₋₂₄ of 3,520 h*ng/mL, and an average Tmax of 9.0 hours (1 hour after the second dose).

### 7.12 Example 12: Evaluation of Pharmacokinetics of Tozadenant in Humans

In a clinical study, tozadenant was administered PO as tablets under fasted conditions, as a single administration at 180 mg. This was an open-label, single-center, randomized, 2-treatment, 2-way crossover bioequivalence study testing 2 different tablet formulation of tozadenant. A modified formulation of tozadenant has been developed for use in Phase 3 studies. The modified formulation for use in Phase 3 studies contains four fewer excipients compared to the Phase 2 formulation. This study, therefore, evaluated the bioequivalence of the Phase 3 formulation and the Phase 2 formulation.

Thirty-six (36) healthy adult, non-tobacco using male and female subjects were enrolled to ensure completion by 32 subjects. Screening of subjects occurred within 28 days prior to the first dose of study drug. In each period, subjects received a single oral dose of tozadenant (180 mg) followed by PK sampling for 120 hours. There was a washout period of at least 7 days between dosing in Periods 1 and 2.

The following PK parameters were calculated from the individual plasma tozadenant concentration-time data. All PK parameters were calculated using Phoenix WinNonlin® Version 6.3. Actual sample times were used for PK parameter calculations. For the calculation of the PK parameters, concentration values below the limit of quantitation (BLQ) were treated as zero before the first quantifiable concentration and as missing thereafter.

The following PK parameters were computed for plasma tozadenant:

| | Label to | |
|---|---|---|
| | be Used | |
| Parameter | in Tables | Definition |
| AUC₀₋ₜ | AUC0-t | The area under the plasma concentration time |
| | | curve, from time 0 to the time of the last |
| | | measurable plasma concentration (tₗₐₛₜ), |
| | | calculated by the linear trapezoidal method. |
| Tₗₐₛₜ | Tlast | Time of the last measurable plasma con- |
| | | centration (last reportable, non-BLQ value). |
| AUC_{0-inf} | AUC0-inf | The area under the plasma concentration versus |
| | | time curve from time 0 to infinity. AUC_{0-inf} was |
| | | calculated as the sum of AUC₀₋ₜ plus the ratio |
| | | of the last measurable plasma concentration |
| | | to the elimination rate constant: |
| | | AUC_{0-inf} = AUC₀₋ₜ + Cₜ/Kₑₗ |
| % AUCₑₓₜ | % | Calculated as (1 − AUC₀₋ₜ/AUC_{0-inf})*100 |
| | AUCext | |
| Cₘₐₓ | Cₘₐₓ | Maximum measured plasma concentration. |
| Tₘₐₓ | Tₘₐₓ | Time of the maximum measured plasma |
| | | concentration. If the maximum value occurred |
| | | at more than one time point, Tₘₐₓ was defined |
| | | as the first time point with this value. |
| kₑₗ | kel | First-order terminal elimination rate constant |
| | | calculated from a semi-log plot of the plasma |
| | | concentration versus time curve. |
| t_{1/2} | t½ | First-order terminal elimination half-life |
| | | calculated as ln(2)/kₑₗ |
| CL/F | CL/F | Apparent total body clearance after |
| | | extravascular administration, calculated as |
| | | Dose/AUC_{0-inf} |
| V_{z}/F | Vz/F | Apparent total volume of distribution after |
| | | extravascular administration, calculated as |
| | | Dose/(AUC_{0-inf} × kₑₗ) |

No value for kₑₗ, t_{1/2}, AUC_{0-inf}, % AUCₑₓₜ, CL/F, or V_{z}/F was reported for cases that did not exhibit a terminal log-linear phase in the concentration versus time profile. If % AUCₑₓₜ was >20%, CL/F and V_{z}/F parameters may not have been reported. AUC and Cₘₐₓ parameters were not to be calculated for subjects with only 2 or less consecutive time points with detectable concentrations.

Blood samples for PK analysis of tozadenant were collected at the following time points:

Predose (Hour 0) and 0.5, 1, 1.5, 2, 3, 4, 5, 6, 7, 8, 10, 12, 16, 24, 36, 48, 72, 96, and 120 hours postdose.

Tozadenant was quantitated using a validated liquid chromatography/tandem mass spectrometry method with a lower limit of quantitation (LLOQ) of 0.00500 μg/mL.

Plasma tozadenant concentrations were detectable through 120 hours postdose in approximately half of the subjects (17/36 subjects following Treatment A [tozadenant Phase 3 formulation] and 19/34 subjects following Treatment B [tozadenant Phase 2 formulation]). There was a less than 12% difference between the highest geometric mean plasma concentrations in the profiles following Treatments A and B, at 0.8762 μg/mL and 0.7744 μg/mL, respectively, observed at 4 hours postdose for both treatments.

The summary of plasma tozadenant PK parameters for the two tablet formulations (Treatments A and B) is presented in the table below:

### Summary of Geometric Mean [CV %] Tozadenant Pharmacokinetic Parameters for Treatments A and B

| | 180 mg Dose of | 180 mg Dose of |
|---|---|---|
| | Tozadenant | Tozadenant |
| Pharmacokinetic | Phase 3 Tablet | Phase 2 Tablet |
| Parameters | (Treatment A, N = 36) | (Treatment B, N = 34*) |
| Cₘₐₓ (μg/mL) | 0.8990 [32.8] | 0.8385 [41.3] |
| Tₘₐₓ (hr) | 4.000 (1.50, 7.00) | 4.001 (2.00, 7.00) |
| AUC₀₋ₜ (μg*hr/mL) | 20.87 [40.6] | 19.56 [43.0] |
| AUC_{0-inf} | 21.27 [40.7] | 19.94 [42.4] |
| (μg*hr/mL) | | |
| % AUCₑₓₜ (%) | 1.189 [98.1] | 1.404 [81.9] |
| t_{1/2} (hr) | 14.727 [40.5] | 15.507 [44.6] |
| kₑₗ (1/hr) | 0.04707 [40.5] | 0.04470 [44.6] |
| CL/F (L/hr) | 8.463 [40.7] | 9.027 [42.4] |
| V_{z}/F (L) | 179.8 [45.7] | 202.0 [53.1] |
| Tₗₐₛₜ (hr) | 96.65 [25.2] | 96.11 [32.4] |

Tₘₐₓ is presented as median (minimum, maximum).
All other parameters are presented as geometric mean, [geometric CV (%)].
Treatment A = 180 mg tozadenant (3 × 60 mg tablets, Phase 3 formulation) at Hour 0 on Day 1.
Treatment B = 180 mg tozadenant (3 × 60 mg tablets, Phase 2 formulation) at Hour 0 on Day 1.
*Subjects 1 and 31 do not have data for Treatment B (Subject 1 withdrew consent prior to Period 2 for personal reasons and Subject 31 was discontinued from the study by the PI on Day −1 of Period 2).

Mean maximum concentrations (Cₘₐₓ) and mean total exposure (AUC_{0-t and} AUC_{0-inf}) to tozadenant were similar for the 2 formulations. Moreover, median Tₘₐₓ values at 4 hours and the mean t_{1/2} at 14.7 and 15.5 hours for Treatments A and B, respectively, were also similar. The mean CL/F was approximately 8.5 L/hr and 9 L/hr following Treatments A and B, respectively, and mean V_{z}/F values were also comparable between the 2 formulations. Mean time of the last measurable concentration was approximately 96 hours for both formulations.

### 7.13 Example 13: Calculation of Predicted Human Dose

The results in the above-described mouse oncology models showed that tozadenant was efficacious in mice at doses of 2-3 mg/kg. Using the PK data above in mice and humans, a human efficacious dose was estimated by identifying a dose in humans that would result in plasma exposures similar to the efficacious dose in the oncology models in mice. The 100 mg/kg/day dose studied for PK in mice and the 180 mg dose studied for PK in humans were the relevant doses used in this estimation. A dose of 100 mg/kg/day in mice resulted in an average AUC of 3,520 hr*ng/mL, and a dose of 180 mg in humans (equivalent to approximately 2.6 mg/kg in an average 70 kg human) resulted in an average AUC of 20,600 hr*ng/mL.

Using linear extrapolation of the dose:AUC relationship in both mice and humans from these doses tested in the PK studies, the dose in humans that would result in a similar AUC as the 3 mg/kg dose in mice that showed efficacy in the mouse oncology models was calculated. Extrapolating from the 10 mg/kg PK dose in mice, the 3 mg/kg efficacious dose would result in a point estimate AUC of approximately 106 hr*ng/mL in mice. Extrapolating from the 5 mg PK dose in humans, the target AUC of 106 hr*ng/mL is predicted to be achieved by a dose in humans of approximately 0.9 mg.

This predicted human efficacious dose for tozadenant for treating cancer based on the measured PK data can be contrasted to the predicted dose that would be calculated by the less informed but standard approach of using body surface area comparisons between mice and humans, whereby the equivalent dose in humans would be calculated by dividing the dose in mice by 12.3 (see, e.g., Nair A B, Jacob S. A simple practice guide for dose conversion between animals and human. J Basic Clin Pharma 2016;7:27-31). This approach would result in a predicted efficacious dose of 0.24 mg/kg in humans, which is equivalent to approximately 17 mg in a typical 70 kg human. Thus, this prediction using the standard method results in a targeted human dose more than an order of magnitude higher than the one informed by PK.

Using the same PK-informed method of calculating as described above for the 3 mg/kg mouse dose, a 2 mg/kg dose in mice results in a target AUC of 71 hr*ng/mL in humans, which is predicted to be achieved by a dose in humans of approximately 0.6 mg.

Accordingly, using human and mouse PK data, the 2-3 mg/kg efficacious dose observed in mice was calculated to correspond to a human dose of 0.6 mg to 0.9 mg. Thus, the maximum daily dose for use in treating cancer in humans is expected to be in the range of about 0.5 mg to about 1.0 mg.

These human doses can be contrasted to the doses that have been investigated in clinical trials for the treatment of Parkinson's disease, where doses of 60 mg and higher, given twice daily (minimum daily dose of 120 mg) have been tested, several orders of magnitude higher than the predicted efficacious doses for oncology based on mouse models.

### 7.14 Example 14: Investigation of the Mechanism Behind SYN-115 Responses in the CT26 Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×105 cells were resuspended in PBS and injected s.c. into BALB/c mice in a 100 μL volume. At day 7 tumors were measured and mice were randomized into distinct treatment groups to ensure that each group had tumors of similar size at the onset of treatment. Mice were treated with SYN-115 (Batch 71236AA006) at the indicated dose or vehicle control once-daily via oral gavage on days 7 to 20. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C.

Seven days post-treatment (14 days post-tumor injection), tumors, spleens and draining lymph nodes (DLN) were excised from the animals. Tumors were weighed before being minced and digested in a solution of DMEM media with 1 mg/ml collagenase type IV and 20 units/mL DNAase for 30 minutes in a 37° C. shaker. Spleens, DLN and digested tumors were then filtered through a 70 μM cell strainer. Cells were centrifuged and resuspended in 400 μL Fc receptor block (2.4G2) and refiltered through a 40 μM cell strainer to form a single cell suspension. 100 μL of solution was aliquoted per FACS cocktail.

For analysis of cytokines, pellets were resuspended in RMPI media (10% fetal bovine serum) with 5 ng/mL PMA, 1 μg/mL ionomycin, 1 μg/mL Golgi Plug and 1 μg/mL GolgiStop, and incubated for 3 hours at 37° C. These human doses can be contrasted to the doses that have been investigated in clinical trials for the treatment of Parkinson's disease, where doses of 60 mg and higher, given twice daily (minimum daily dose of 120 mg) have been tested, several orders of magnitude higher than the predicted efficacious doses for oncology based on mouse models.

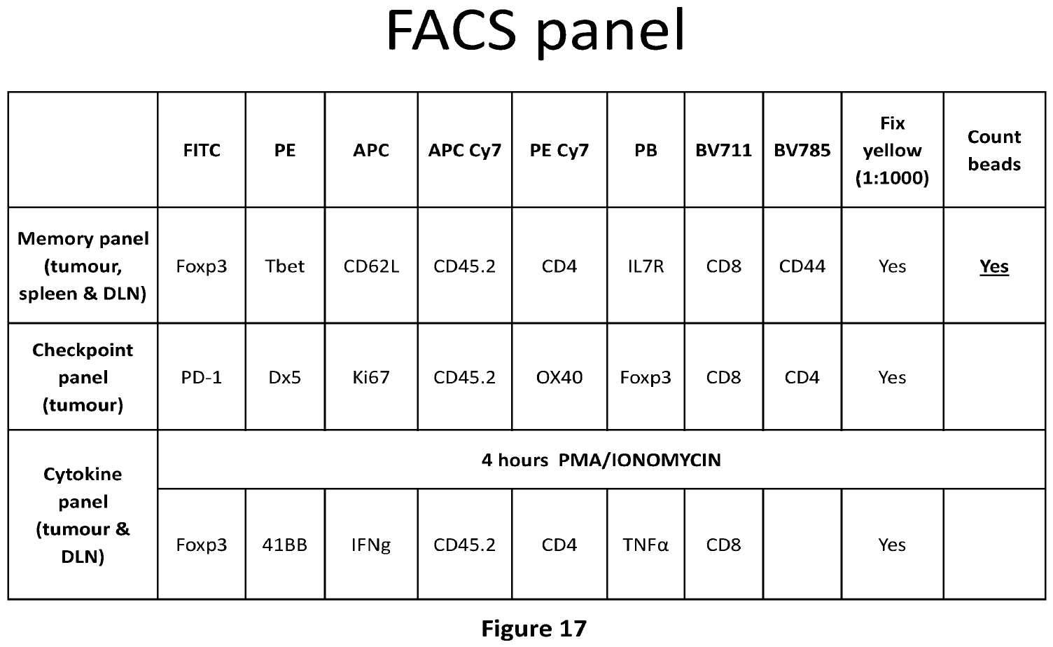

Cells were stained with the cocktails presented in FIG. 17.

Samples were run on the BD LSRFortessa and data analyzed with FlowJo Version 10.

### Results:

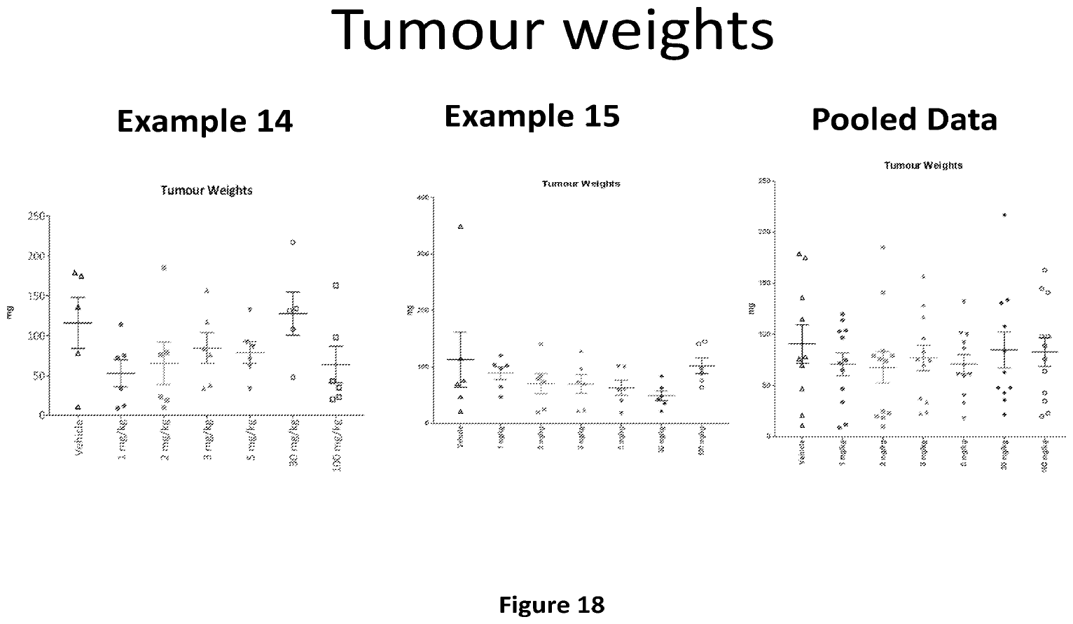

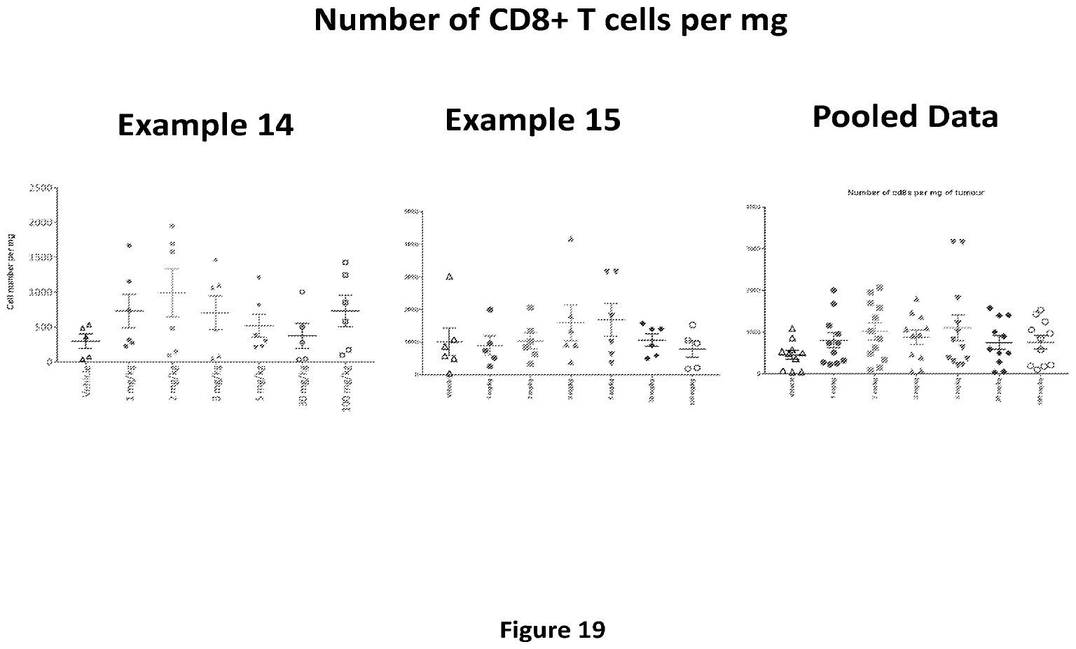

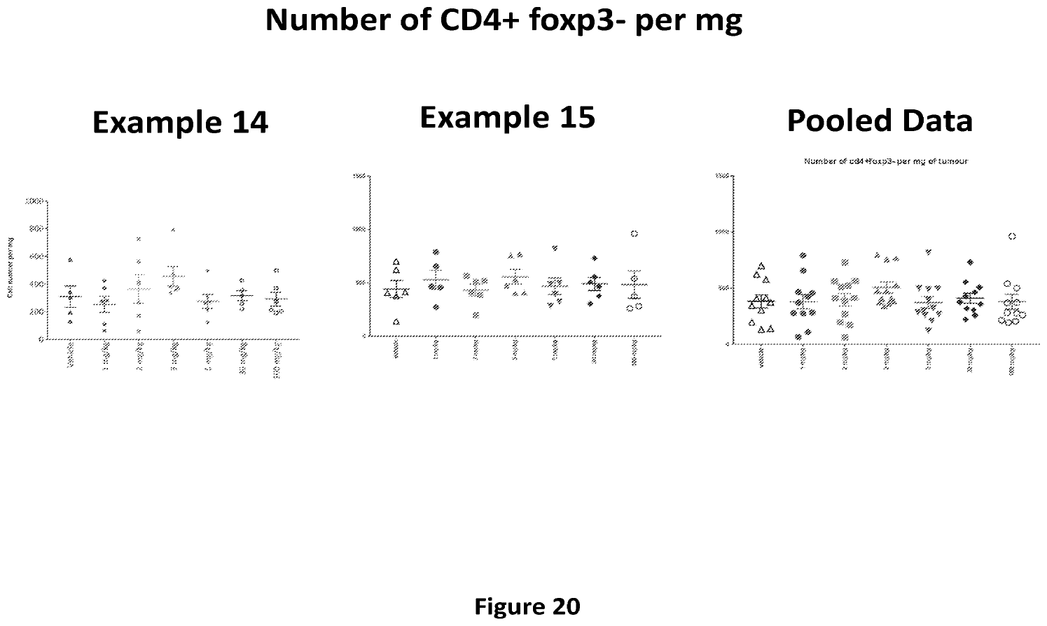

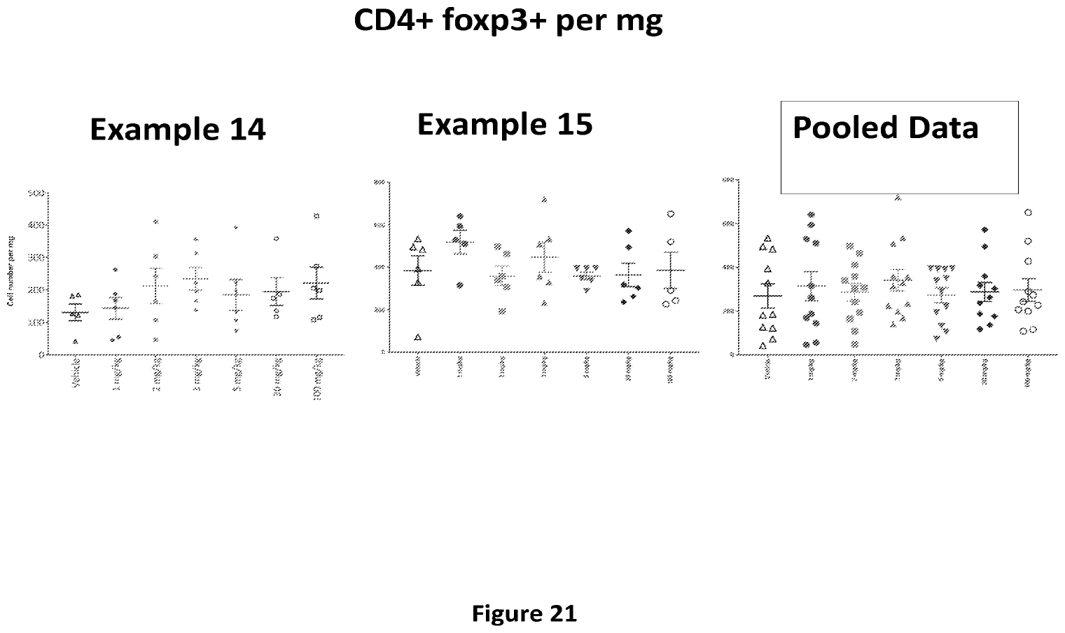

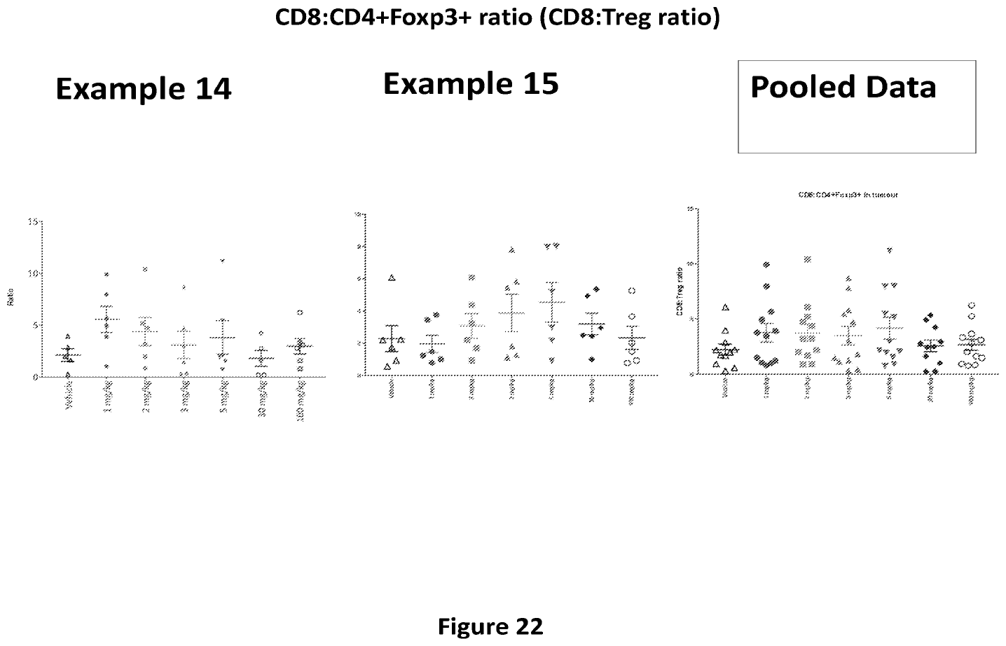

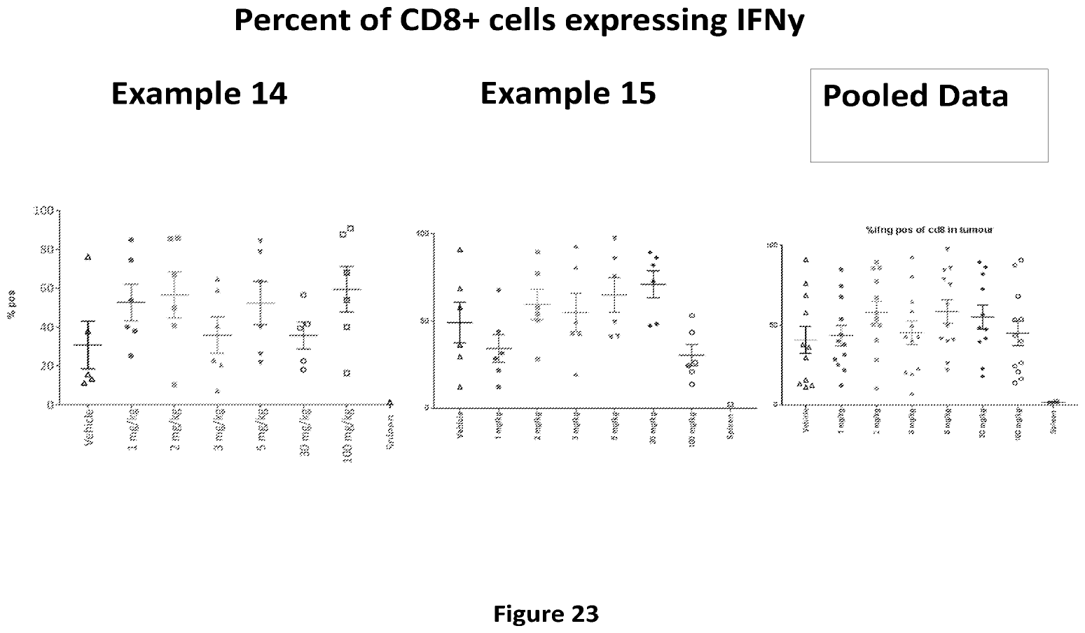

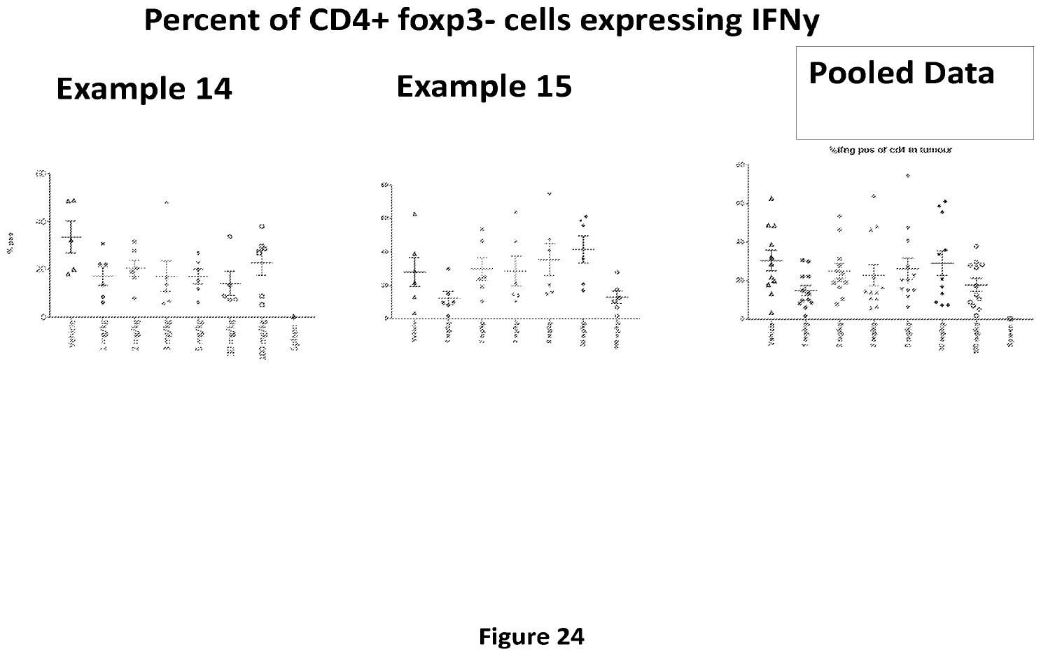

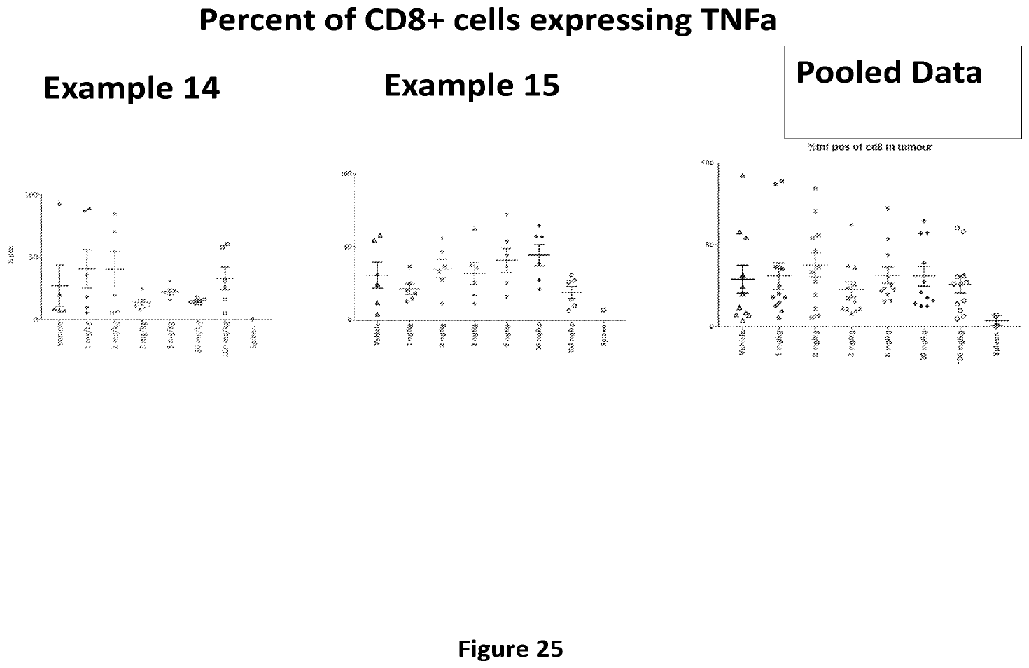

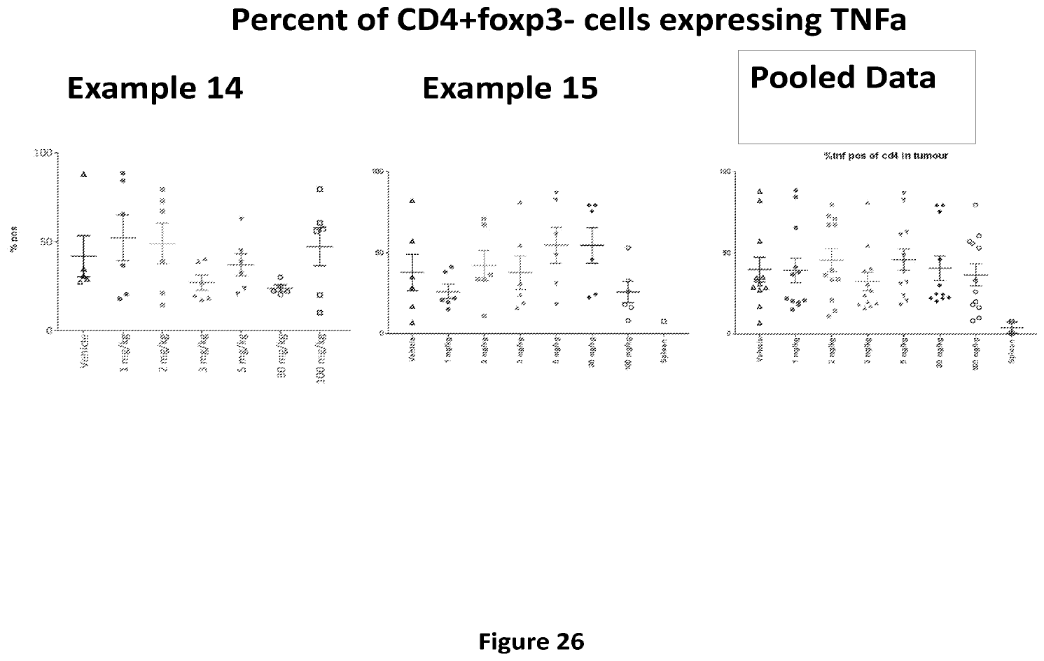

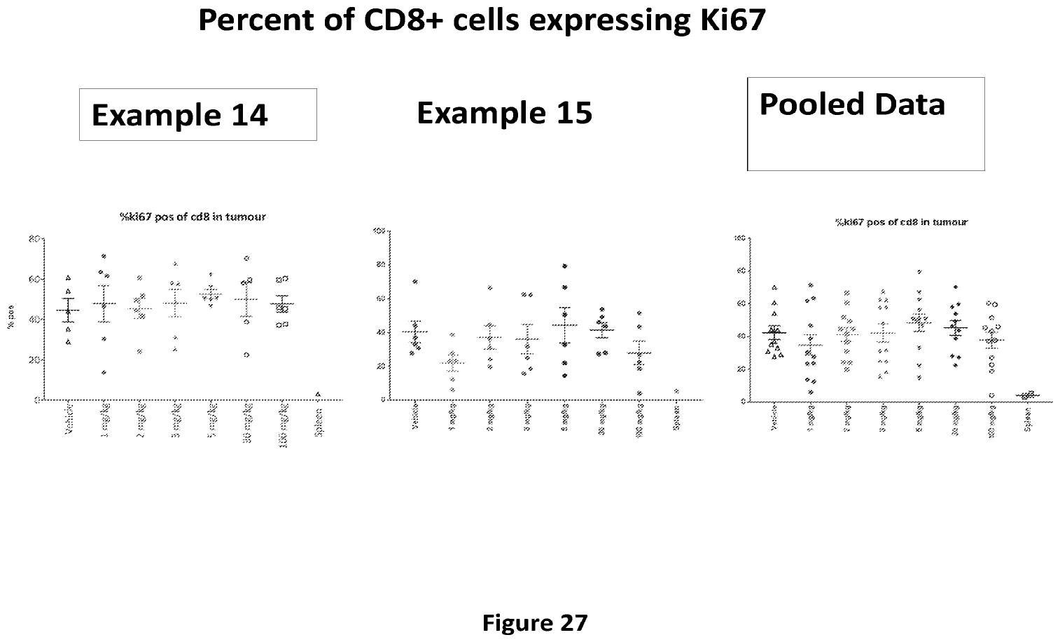

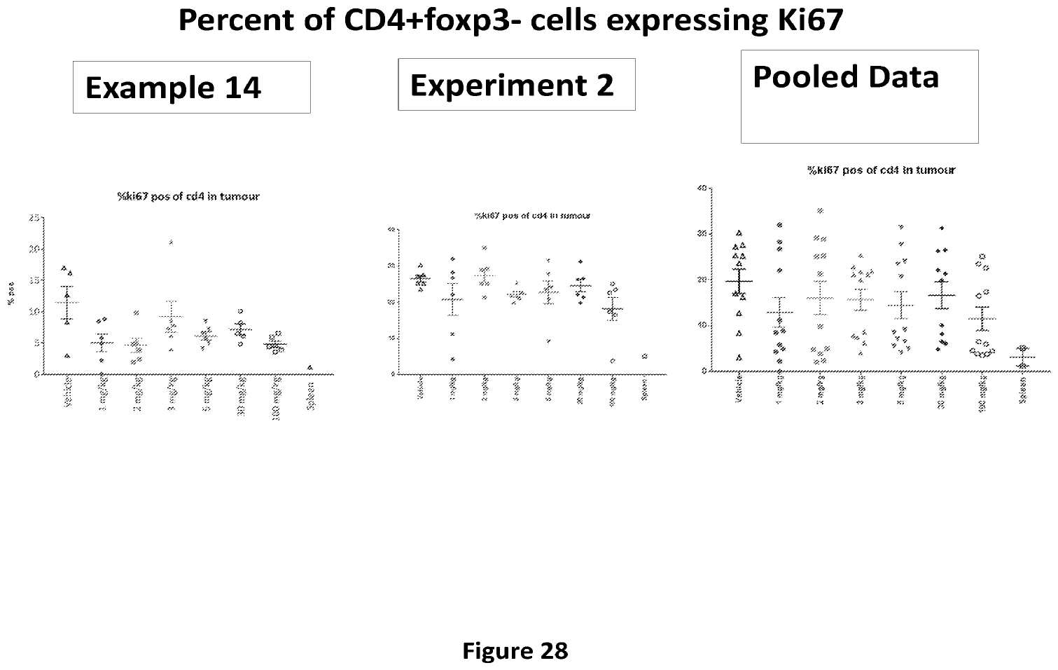

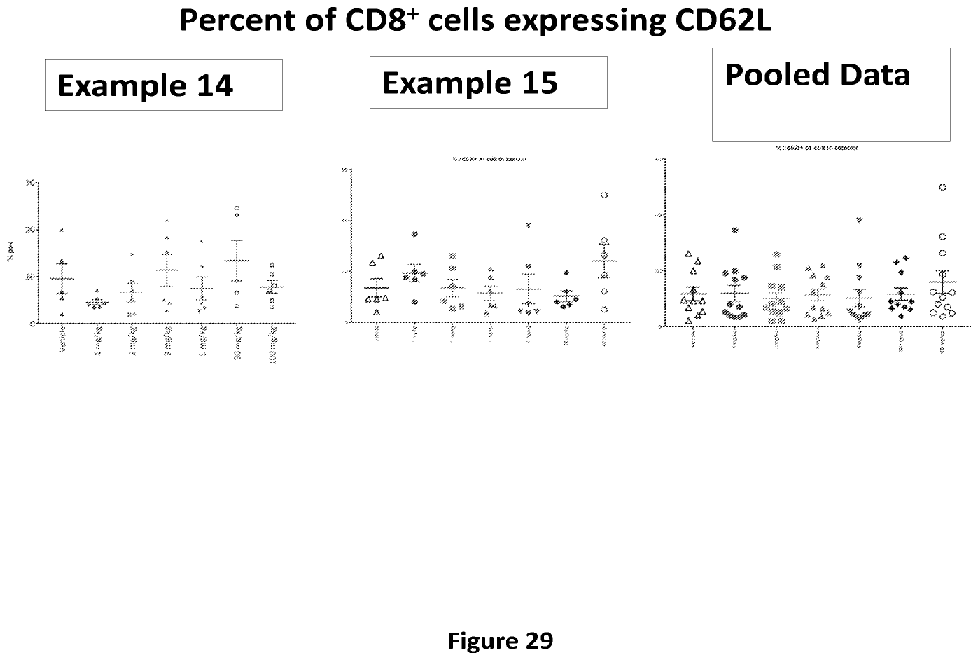

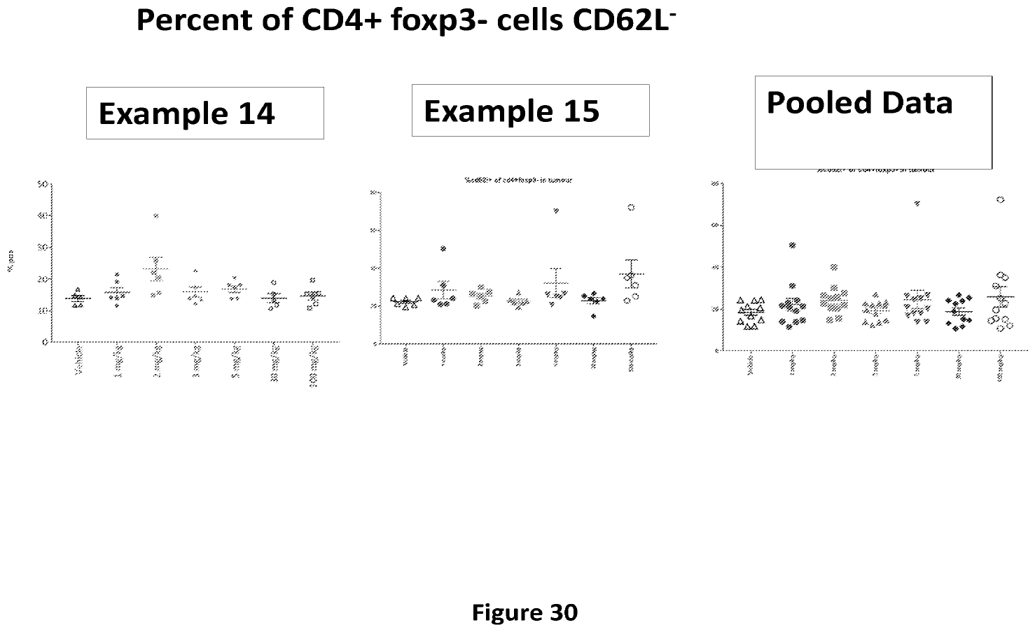

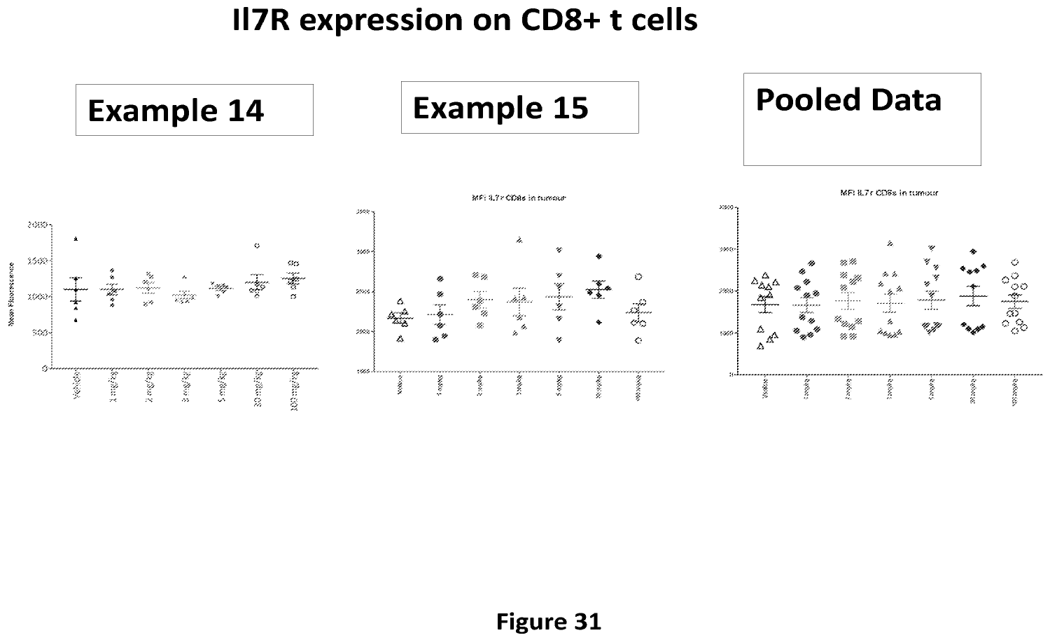

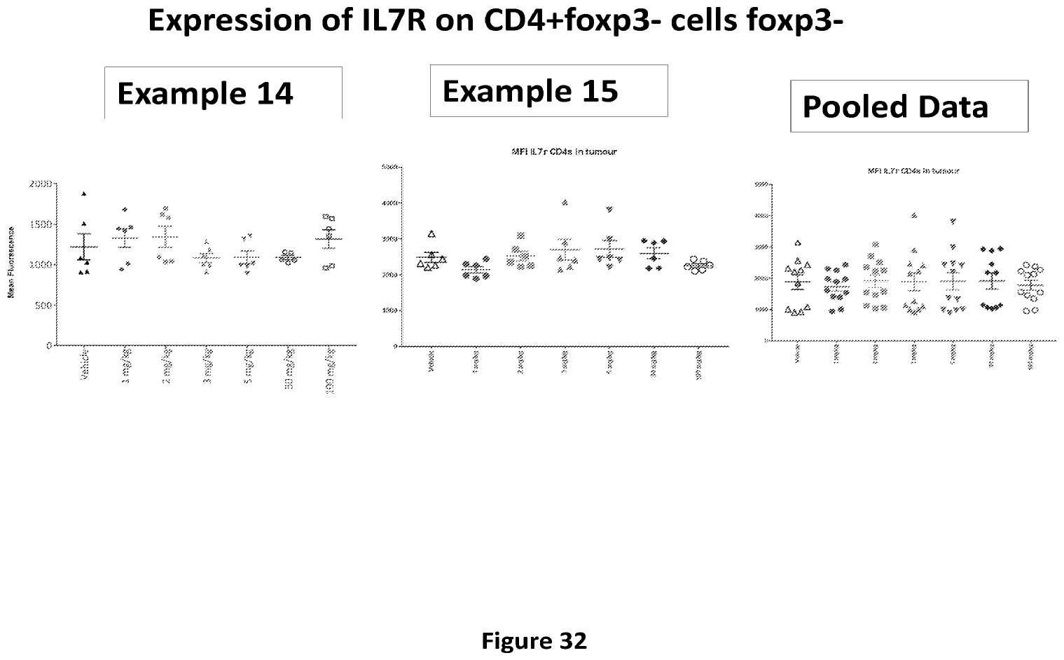

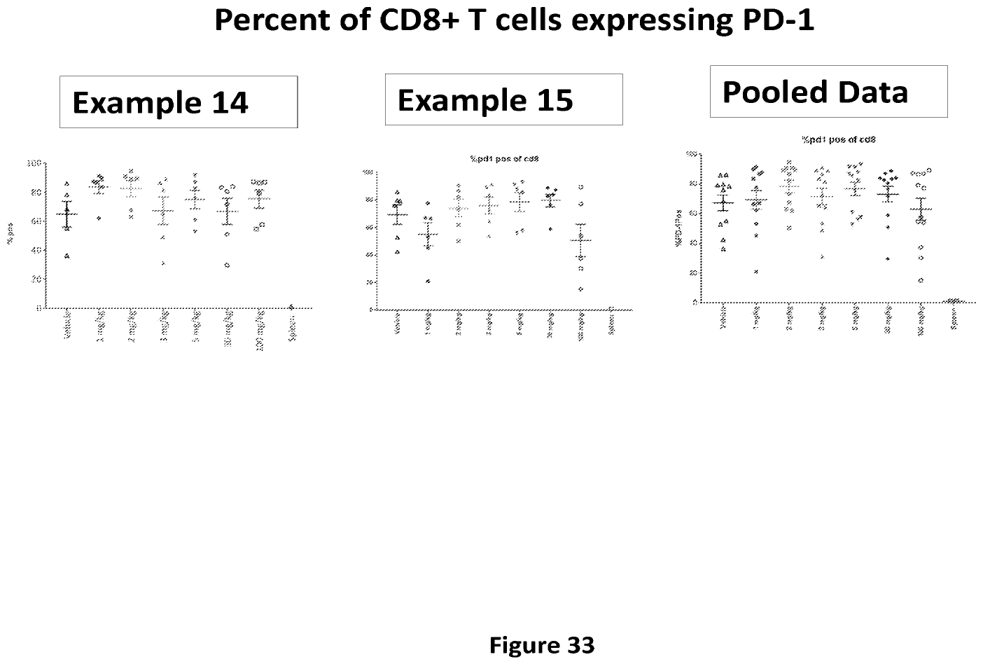

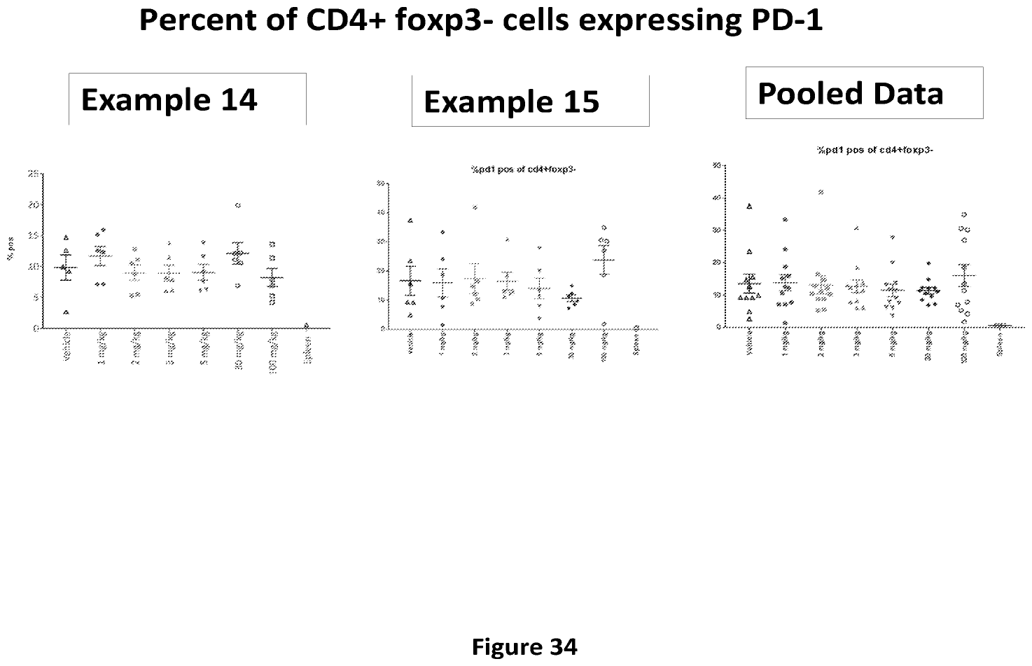

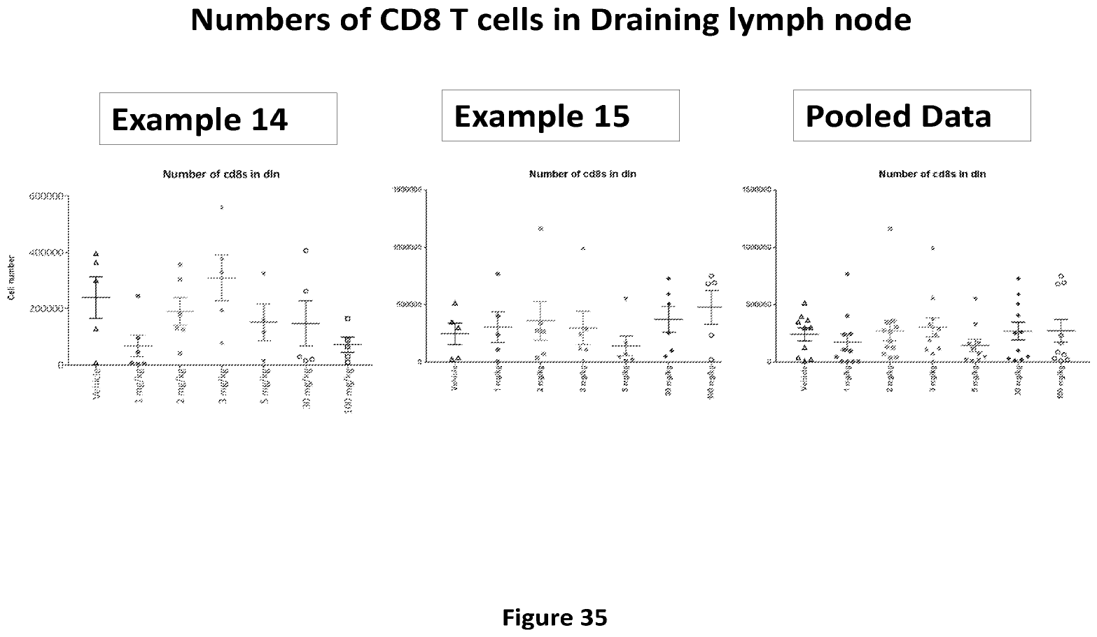

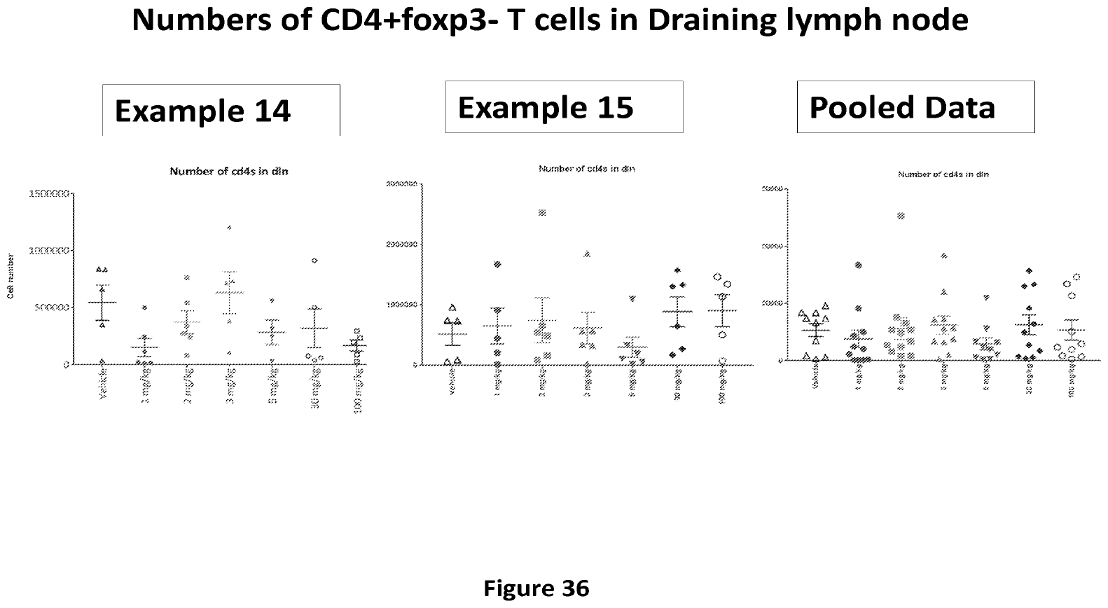

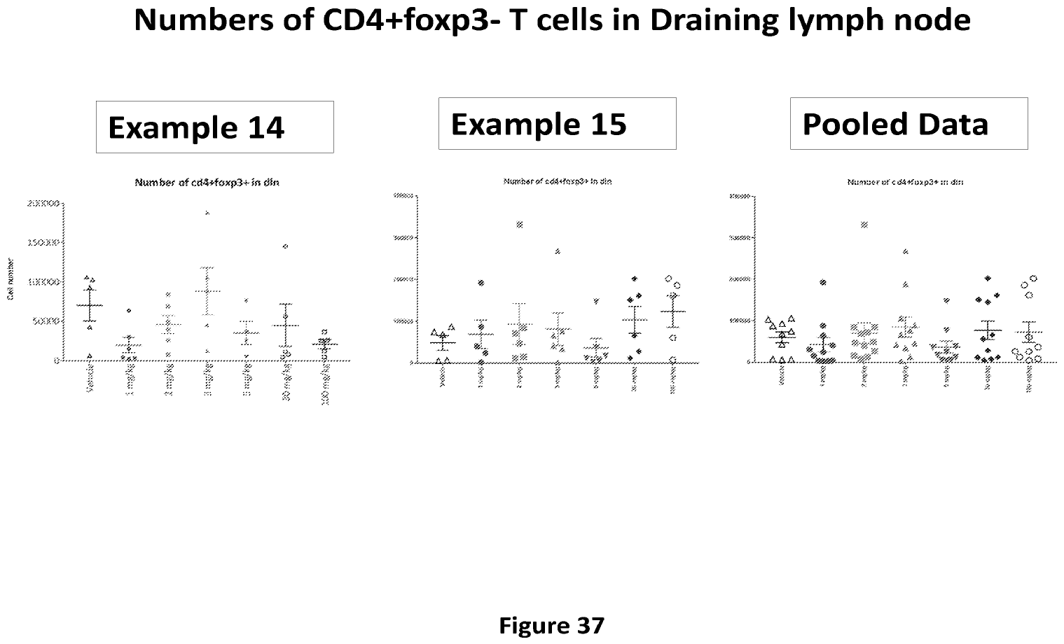

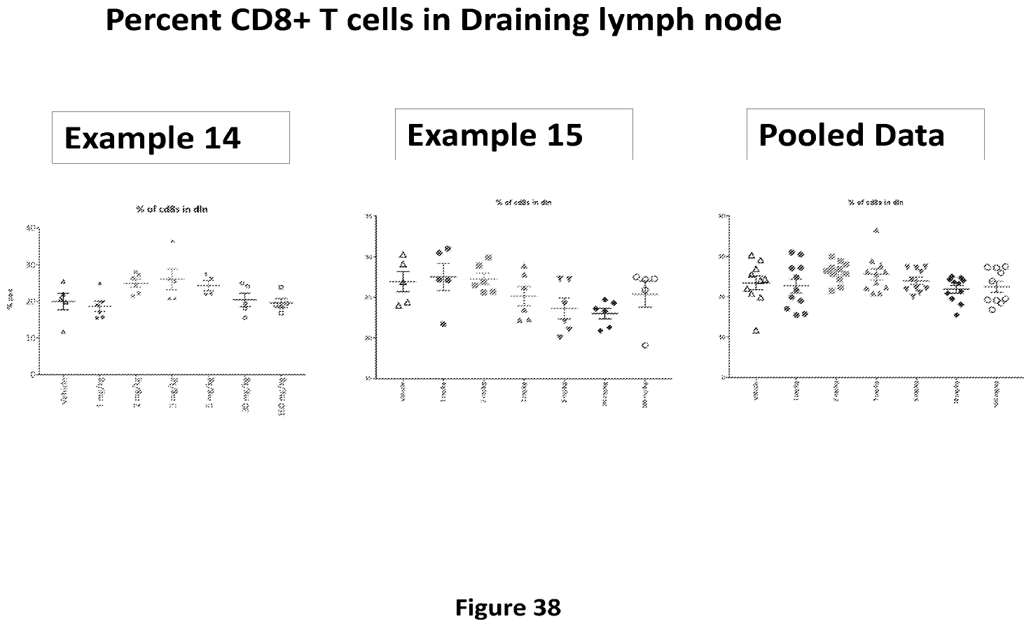

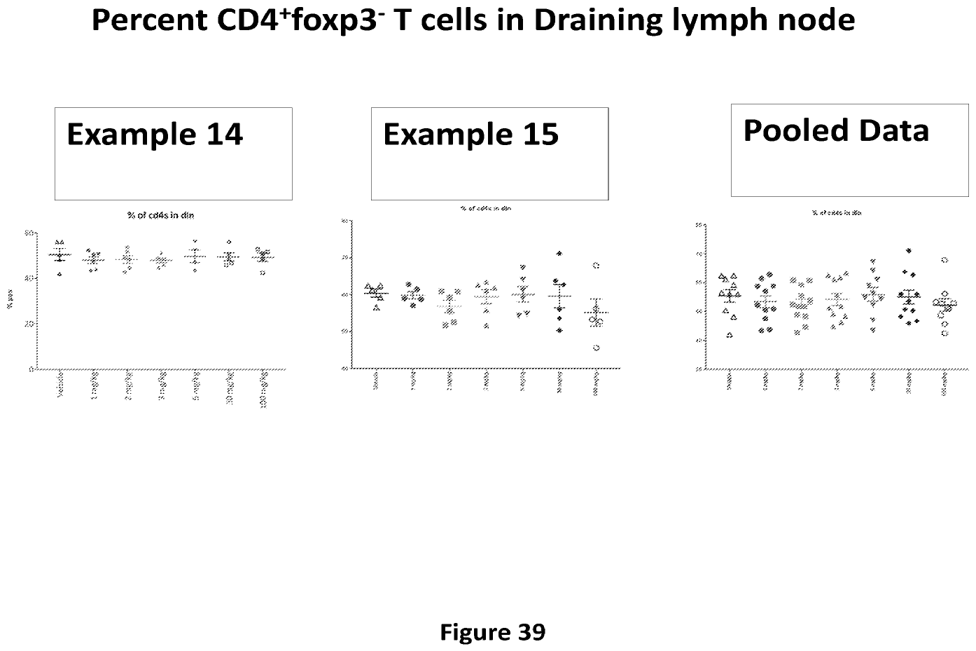

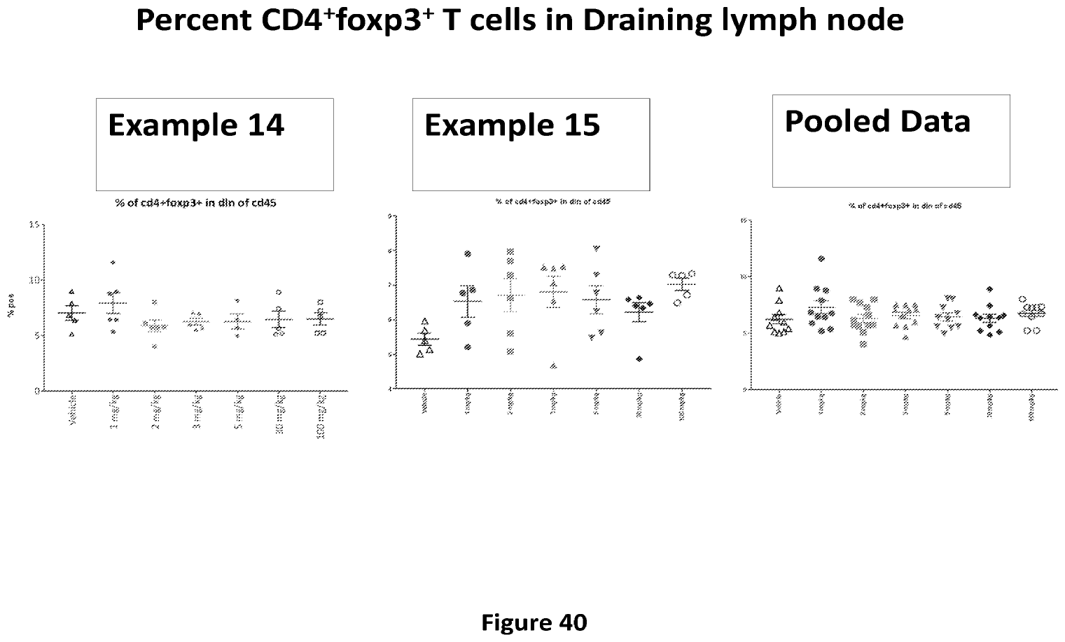

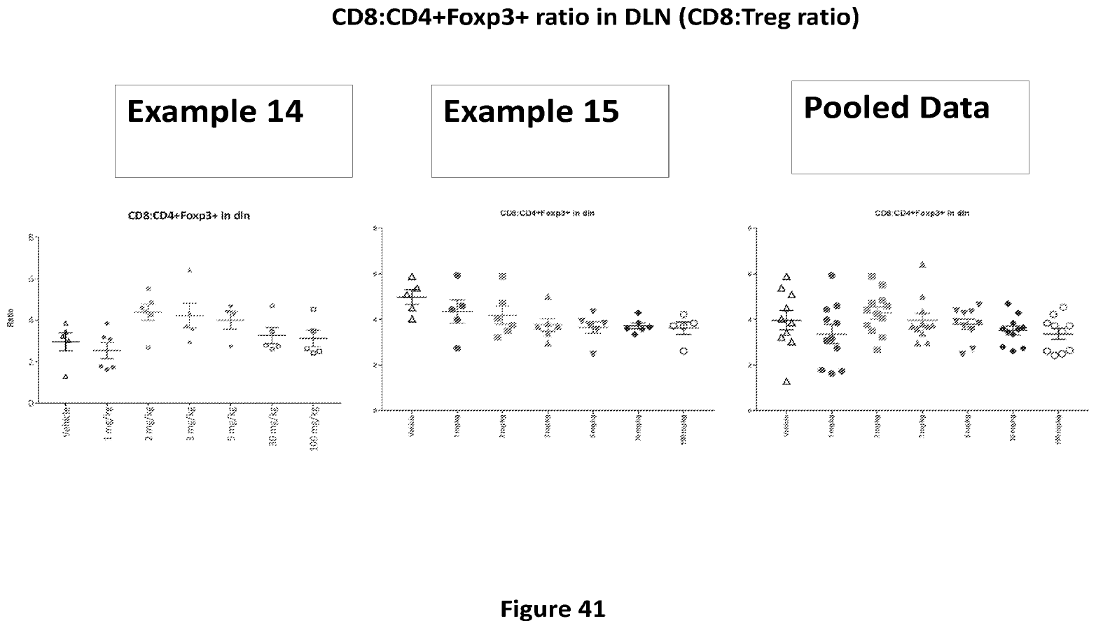

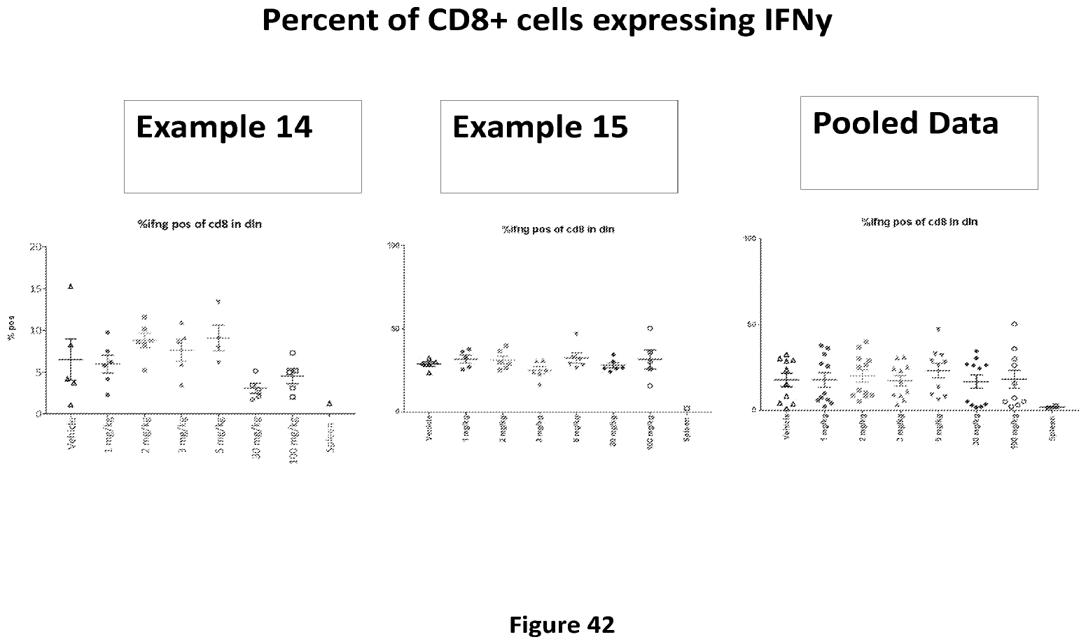

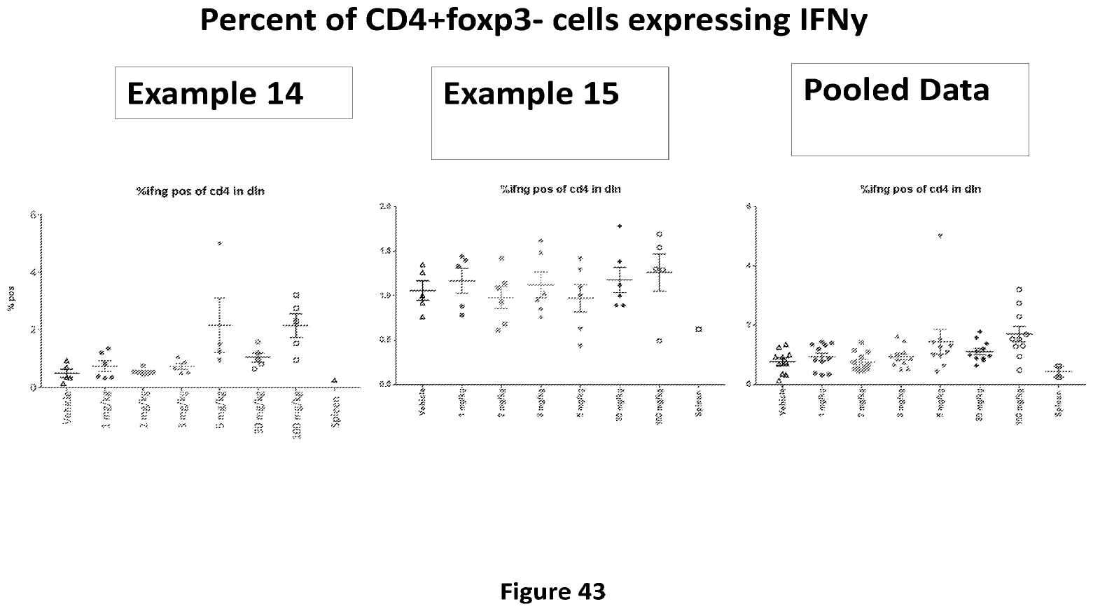

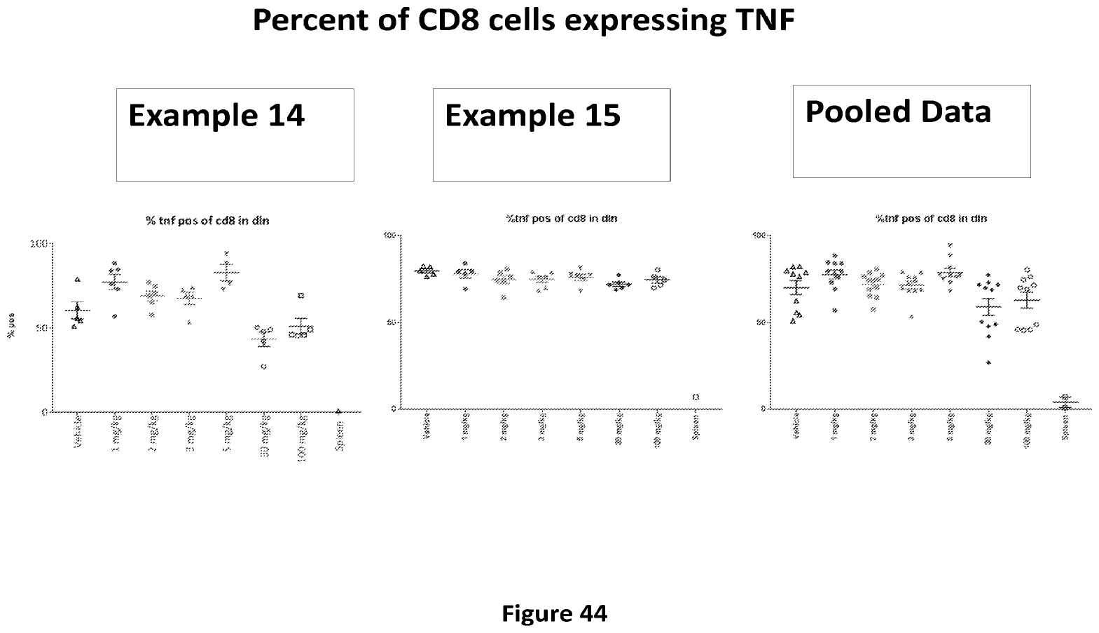

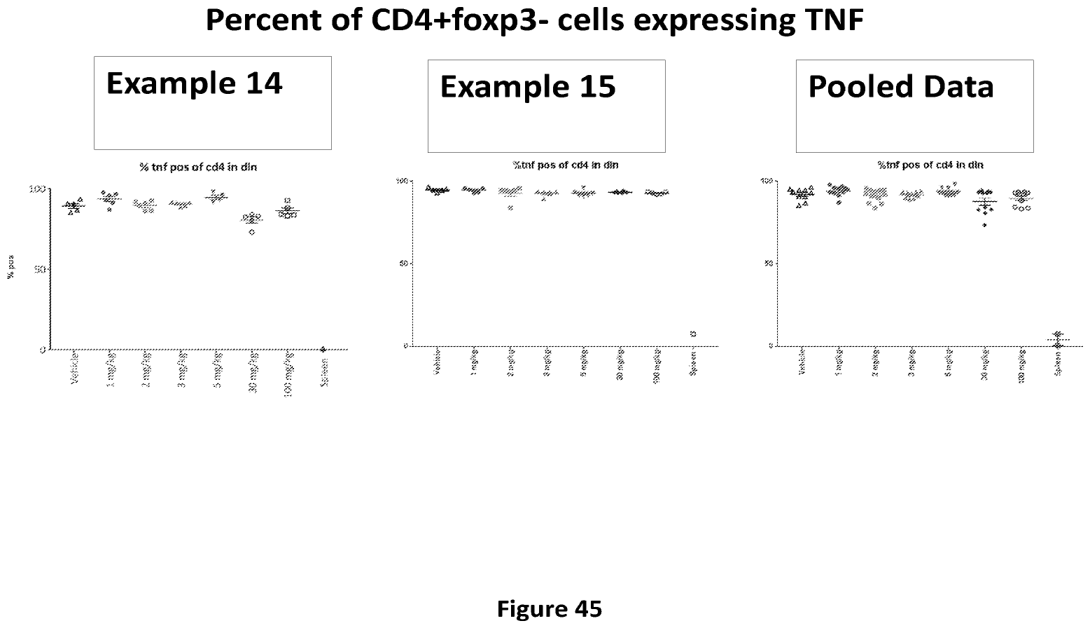

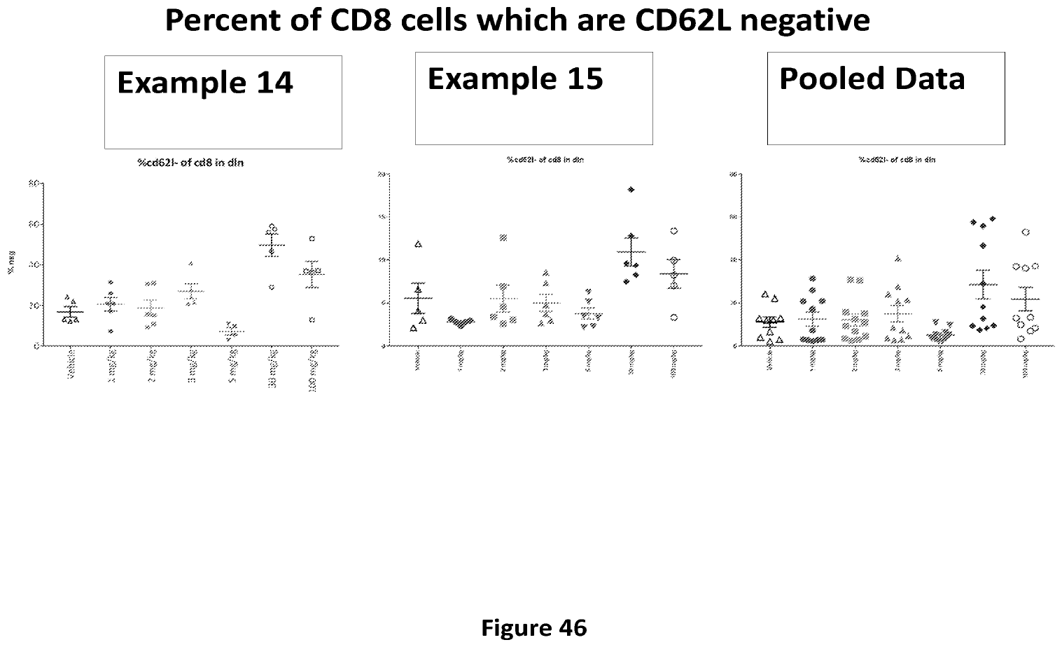

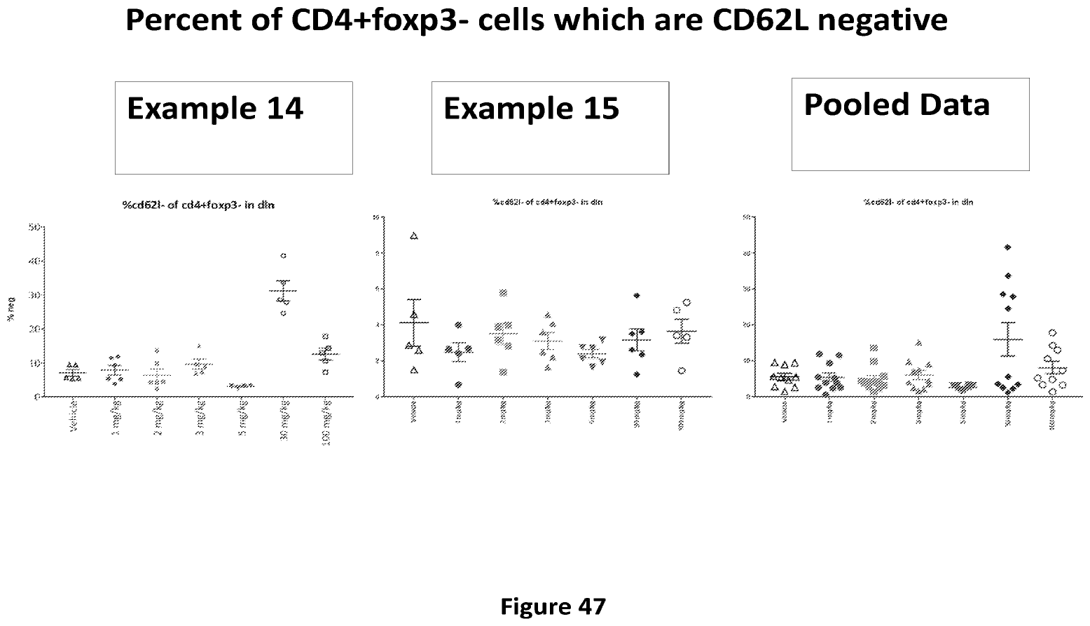

Results are presented in FIGS. 18-47.

### Conclusions

Low dose SYN-115 (1-10 mg/kg) led to an increase in CD8⁺IFNγ⁺ T cells compared to high dose SYN-115 (100 mg/kg). IFNγ is a potent anti-tumor cytokine. This likely accounts for the increased therapeutic effect of SYN-115 at low doses. Furthermore, CD4⁺ T cell function was inhibited by high dose SYN-115 that may also be a contributing factor for the differences observed in the therapeutic effect shown in Example 7. A direct repeat of this experiment was conducted as described in Example 15.

### 7.15 Example 15: Investigation of the Mechanism Behind SYN-115 Responses in the CT26 Model

### Methods:

The BALB/c mouse colon carcinoma cell line CT26 was cultured in RPMI supplemented with 10% FCS, GlutaMAX, and penicillin/streptomycin. Tumor cells are periodically verified to be Mycoplasma negative by the Victorian Infectious Diseases References Lab (Melbourne, VIC, Australia) by PCR analysis. 1×10⁵ cells were resuspended in PBS and injected s.c. into BALB/c mice in a 100 μL volume. At day 7 tumors were measured and mice were randomized into distinct treatment groups to ensure that each group had tumors of similar size at the onset of treatment. Mice were treated with SYN-115 (Batch 71236AA006) at the indicated dose or vehicle control once-daily via oral gavage on days 7 to 20. To calculate a dose of SYN-115 mouse weight was estimated to be 20 g per mouse. Therefore, 100 mg/kg=2 mg per mouse in 200 μL, which is equivalent to 10 mg/ml. To make up 40 mL, 400 milligrams of SYN-115 was weighed and added into a Falcon tube, then 1 mL of Tween 80 was added and mixed into a paste and then 39 mL of 1% Methyl Cellulose was added. The solution was vortexed and then left on a rotating mixer for at least 2 hours and maintained at 4° C.

Seven days post-treatment (14 days post-tumor injection), tumors, spleens and draining lymph nodes (DLN) were excised from the animals. Tumors were weighed before being minced and digested in a solution of DMEM media with 1 mg/ml collagenase type IV and 20 units/mL DNAase for 30 minutes in a 37° C. shaker. Spleens, DLN and digested tumors were then filtered through a 70 μM cell strainer. Cells were centrifuged and resuspended in 400 μL Fc receptor block (2.4G2) and refiltered through a 40 μM cell strainer to form a single cell suspension. 100 μL of solution was aliquoted per FACS cocktail.

For analysis of cytokines, pellets were resuspended in RIVIPI media (10% fetal bovine serum) with 5 ng/mL PMA, 1 μg/mL ionomycin, 1 μg/mL Golgi Plug and 1 μg/mL GolgiStop, and incubated for 3 hours at 37° C.

Cells were stained with the cocktails presented in FIG. 17.

Samples were run on the BD LSRFortessa and data analyzed with FlowJo Version 10.

### Results:

Results are presented in FIGS. 18-47.

### Conclusions

Taken together, Examples 14 and 15 showed that low-dose SYN-115 treatment is associated with enhanced anti-tumor effects. This was evident in the increased numbers of CD8+ T cells as well as their proliferation, IFNγ production and PD-1 expression. Surprisingly, SYN-115 treatment appeared deleterious to the CD4⁺foxp3⁻ population. These data support the use of SYN-115 in immunocancer therapy particularly in combination with anti-PD-1 or with reagents designed to enhance CD4⁺ T cell activity, for example anti-OX-40.

### Summary of Examples

The above Examples have shown that SYN-115 has single agent activity in the melanoma lung metastasis model (B16F10 CD73) and in the localized subcutaneous cancer setting using the CT26 colon carcinoma syngeneic cell line. The majority of studies were conducted using a dose of 30-100 mg/kg SYN-115, and using these doses, the 30 mg/kg exhibited superior anti-tumor effects and synergized with the checkpoint inhibitor anti-PD-1. Interestingly, in later experiments (e.g. Example 7), the therapeutic effects were even greater when lower doses of SYN-115 were used (3-5 mg/kg). Immunological analysis using FACS (Examples 14 and 15) confirmed that lower doses of SYN-115 were more effective than higher doses in enhancing CD8⁺ T cell function in particular IFN-γ production which is a key mediator of the anti-tumor response. An unexpected result was the finding that higher doses of SYN-115 appeared to inhibit CD4⁺ T cell function.

### INCORPORATION BY REFERENCE

Various references such as patents, patent applications, and publications are cited herein, the disclosures of which are hereby incorporated by reference herein in their entireties.

What is claimed is:

1. A method of treating cancer in a human patient in need thereof, said method comprising orally administering to the patient a maximum daily dose in the range of about 0.5 to about 1.0 mg of tozadenant or a molar-equivalent amount thereof of a pharmaceutically acceptable salt of tozadenant.

2. The method of claim 1, wherein the administering of said maximum daily dose is daily.

3. The method of claim 1, wherein the administering of said maximum daily dose is every other day.

4. The method of claim 1, wherein the administering of said maximum daily dose is every 3-4 days.

5. The method of claim 1, wherein the administering of said maximum daily dose is weekly.

6. The method of claim 1, wherein the administering of said maximum daily dose is every other week.

7. The method of any one of claims 1-6, wherein the dose is about 0.5 mg.

8. The method of any one of claims 1-6, wherein the dose is about 0.6 mg.

9. The method of any one of claims 1-6, wherein the dose is about 0.7 mg.

10. The method of any one of claims 1-6, wherein the dose is about 0.8 mg.

11. The method of any one of claims 1-6, wherein the dose is about 0.9 mg.

12. The method of any one of claims 1-6, wherein the dose is about 1.0 mg.

13. The method of any one of claims 1-6, wherein the dose is 1.0 mg.

14. The method of any one of claims 1-6, wherein the dose is 0.5 mg.

15. The method of any one of claims 1-14, wherein the dose is administered in two separate administrations on the same day.

16. The method of claim 15, wherein the separate administrations are administered about once every 12 hours.

17. The method of claim 15 or 16, wherein the separate administrations are each about 50% of the daily dose.

18. The method of any one of claims 1-17, wherein the cancer is a solid tumor.

19. The method of any one of claims 1-17, wherein the cancer is breast cancer.

20. The method of any one of claims 1-17, wherein the cancer is colon cancer.

21. The method of any one of claims 1-17, wherein the cancer is lung cancer.

22. The method of any one of claims 1-17, wherein the cancer is melanoma.

23. The method of any one of claims 1-22, wherein tozadenant or a pharmaceutically acceptable salt thereof is formulated as a capsule, a pill or a tablet.

24. The method of claim 23, wherein tozadenant or a pharmaceutically acceptable salt thereof is formulated as a tablet.

25. The method of any one of claims 1-24, wherein tozadenant is the only cancer therapeutic administered to said human patient during the course of treatment with tozadenant.

26. The method of any one of claims 1-24, which further comprises concomitantly treating the patient with a PD-1 inhibitor.

27. The method of claim 26, wherein the PD-1 inhibitor is an anti-PD-1 monoclonal antibody.

28. The method of any one of claims 1-27, wherein tozadenant or a pharmaceutically acceptable salt thereof is combined with a pharmaceutically acceptable carrier in a pharmaceutical composition.

29. The method of any one of claims 1-28, wherein tozadenant is administered.

30. A unit dosage pharmaceutical composition comprising about 0.5 to about 1.0 mg of tozadenant or a molar-equivalent amount thereof of a pharmaceutically acceptable salt of tozadenant, and a pharmaceutically acceptable carrier.

31. The unit dosage pharmaceutical composition of claim 30 comprising about 0.5 mg of tozadenant or a molar-equivalent amount thereof of a pharmaceutically acceptable salt of tozadenant.

32. The unit dosage pharmaceutical composition of claim 30 comprising about 0.6 mg of tozadenant or a molar-equivalent amount thereof of a pharmaceutically acceptable salt of tozadenant.

33. The unit dosage pharmaceutical composition of claim 30 comprising about 0.7 mg of tozadenant or a molar-equivalent amount thereof of a pharmaceutically acceptable salt of tozadenant.

34. The unit dosage pharmaceutical composition of claim 30 comprising about 0.8 mg of tozadenant or a molar-equivalent amount thereof of a pharmaceutically acceptable salt of tozadenant.

35. The unit dosage pharmaceutical composition of claim 30 comprising about 0.9 mg of tozadenant or a molar-equivalent amount thereof of a pharmaceutically acceptable salt of tozadenant.

36. The unit dosage pharmaceutical composition of claim 30 comprising about 1.0 mg of tozadenant or a molar-equivalent amount thereof of a pharmaceutically acceptable salt of tozadenant.

37. The unit dosage pharmaceutical composition of claim 30 comprising 1.0 mg of tozadenant or a molar-equivalent amount thereof of a pharmaceutically acceptable salt of tozadenant.

38. The unit dosage pharmaceutical composition of claim 30 comprising 0.5 mg of tozadenant or a molar-equivalent amount thereof of a pharmaceutically acceptable salt of tozadenant.

39. The unit dosage pharmaceutical composition of any one of claims 30-38, wherein the unit dosage pharmaceutical composition is formulated as a capsule, a pill or a tablet.

40. The unit dosage pharmaceutical composition of claim 39 which is formulated as a tablet.

41. A kit comprising in one or more containers a plurality of the unit dosage pharmaceutical composition of any one of claims 30-40.

42. A method of treating cancer in a human patient in need thereof, said method comprising orally administering to the patient the unit dosage pharmaceutical composition of any one of claims 30-40.

43. The method of claim 42, wherein the administering of said unit dosage pharmaceutical composition is once daily.

44. The method of claim 42, wherein the administering of said unit dosage pharmaceutical composition is once every other day.

45. The method of claim 42, wherein the administering of said unit dosage pharmaceutical composition is once per week.

46. The method of claim 42, wherein the administering of said unit dosage pharmaceutical composition is once every 3-4 days.

47. The method of claim 42, wherein the administering of said unit dosage pharmaceutical composition is once every other week.

48. The method of any one of claims 42-47, wherein the cancer is a solid tumor.

49. The method of any one of claims 42-47, wherein the cancer is breast cancer.

50. The method of any one of claims 42-47, wherein the cancer is colon cancer.

51. The method of any one of claims 42-47, wherein the cancer is lung cancer.

52. The method of any one of claims 42-47, wherein the cancer is melanoma.

53. The method of any one of claims 42-52, wherein tozadenant is the only cancer therapeutic administered to said human patient during the course of treatment with tozadenant.

54. The method of any one of claims 42-52, which further comprises concomitantly treating the patient with a PD-1 inhibitor.

55. The method of claim 54, wherein the PD-1 inhibitor is an anti-PD-1 monoclonal antibody.

56. A method of treating cancer in a human patient in need thereof, said method comprising orally administering to the patient an amount of tozadenant or a pharmaceutically acceptable salt thereof that provides an AUC at steady state of about 71 hr*ng/mL to about 106 hr*ng/mL in the patient.

57. The method of claim 56, wherein the AUC at steady state is about 106 hr*ng/mL.

58. The method of claim 56, wherein the AUC at steady state is about 71 hr*ng/mL.

59. Use of a maximum daily dose in the range of about 0.5 to about 1.0 mg of tozadenant or a molar-equivalent amount thereof of a pharmaceutically acceptable salt of tozadenant for the manufacture of a medicament for treating cancer in a human patient.

60. The use of claim 59, wherein said medicament is formulated for oral administration.

61. Tozadenant or a pharmaceutically acceptable salt thereof for use in treating cancer in a human at a maximum daily oral dose in the range of about 0.5 to about 1.0 mg of tozadenant or a molar-equivalent amount thereof of a pharmaceutically acceptable salt of tozadenant.

62. A method for inhibiting metastasis of a malignant tumor in a human patient in need thereof, said method comprising orally administering to the patient a maximum daily dose in the range of about 0.5 to about 1.0 mg of tozadenant or a molar-equivalent amount thereof of a pharmaceutically acceptable salt of tozadenant.
